(19) United States
(12) Patent Application Publication
(10) Pub. No.: US 2008/0166785 A1
(43) Pub. Date: Jul. 10, 2008

(54) Polynucleotides allowing the expression and secretion of recombinant HBsAg virus-like particles containing a foreign peptide, their production and use

(76) Inventors: Monica Sala-Schaeffer, Paris (FR); Marie Michel, Ragny Les Marceaux (FR); Simon Wain-Hobson, Montigny-le-Bretonneux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/889,218

(22) Filed: Aug. 9, 2007

Related U.S. Application Data
(60) Provisional application No. 60/837,912, filed on Aug. 16, 2006.

(51) Int. Cl. C12N 7/04; C07H 21/04; C12N 15/00; C12N 5/00; C07K 16/00

(52) U.S. Cl. 435/236; 536/23.72; 435/320.1; 435/325; 530/350

(57) ABSTRACT

The hepatitis B surface antigen (HBsAg) can assemble into sub-virion virus like particles (VLPs). Vectors comprised of polynucleotides, identified as GA1xFlag-M and GA3xFlag-M, are provided for the expression of foreign proteins in such VLPs, and for release of the VLPs from host cells containing the vectors. In one example, an HIV-1 polyepitope-HBsAg recombinant fusion protein assembled into VLPs and was efficiently secreted.

### CROSS-REFERENCE TO RELATED APPLICATIONS

This application is based on and claims the benefit of U.S. Provisional Application No. 60/837,912, filed Aug. 16, 2006 (Attorney Docket No. 03495.6116) The entire disclosure of this application is relied upon and incorporated by reference herein.

### FIELD OF THE INVENTION

This invention relates to polynucleotides for the expression of recombinant hepatitis B surface antigen (HBsAg) virus-like particles (VLPs) and to the secretion of the particles from host cells. The recombinant HBsAg virus-like particles can contain a foreign peptide or polypeptide, such as foreign amino acid residues of a pathogen. The HBsAg virus-like particles are particularly useful in immunogenic compositions and as vaccines.

### BACKGROUND OF THE INVENTION

Many viral structural proteins have the intrinsic ability to assemble into virus-like particles (VLPs) independently of nucleic acids. VLPs can elicit potent anti-viral humoral and cellular immune responses directed against viruses they derive from (10, 24, 36, 37). They are efficiently taken up, rapidly internalised, and processed by antigen presenting cells (APCs) of myeloid origin, leading to MHC class I-associated antigen cross-presentation (1,17, 33-35, 38). Indeed, MHC class I cross-presentation of VLP epitopes by APCs can be exploited to induce anti-viral CD8+cytotoxic T lymphocyte (CTL) responses. VLPs are powerful antigen delivery systems, the most developed examples being hepatitis B surface antigen (HBsAg), the yeast Ty retrotransposon structural protein “a” (Tya), the VP2 capsid protein of porcine parvovirus (PPV), and the papillomavirus capsid L1 protein. The generation of recombinant VLPs bearing relevant antigens opens up the way to the development of bivalent vaccine candidates (19, 21, 30).

Even though the three envelope proteins of hepatitis B virus (HBV), (large, middle, and small: L, M and S protein, respectively) are encoded by a sole open reading frame (orf), they are encoded by distinct regions of the orf as a result of two different mRNA transcripts (L by preS1+pre S2+S regions of a first mRNA; M and S by pre S2+S regions and S region of the second mRNA, respectively). The preS2 translation initiation codon is less efficient than the S region one (HBsAg) meaning that the mRNA is bicistronic (14).

HBsAg carries all the information necessary for membrane translocation, particle assembly, and secretion from mammalian cells (5). Substitutions within HBsAg that impair VLPs assembly are generally characterized by HBsAg accumulation in the endoplasmic reticulum (ER) and Golgi apparatus (8).

By fusing foreign DNA to the S protein gene, HBsAg has been used as carrier for a wide panel of antigens (12, 19, 21, 27, 30). In a notable example, a series of 13 HIV-1 epitopes restricted by the HLA-A*0201 class I allele, which is present at ˜15-30% of Black, Caucasian, and Oriental populations, was incorporated into the preS2 region as a polyepitope (polHIV-1) fused to HBsAg. Although the study reported the induction of HIV-1 specific CTL responses by DNA vaccination (12) of humanised HLA-A*0201 transgenic mice (11), it was not shown whether the recombinant HBsAg actually formed VLPs.

Thus, there exists a need in the art for vectors for the expression of recombinant peptides or polypeptides, such as HIV polyepitopes, compatible with VLPs formation and secretion of recombinant VLPs from host cells. Preferably, recombinant VLPs secretion should result in the induction of robust neutralising anti-HBsAg humoral immune responses and the enhancement of the activation state of foreign, sequence specific CD8⁺T lymphocytes so that the VLPs can be employed in therapeutic applications.

### SUMMARY OF THE INVENTION

A previous HLA.A2.1-restricted HIV-1 polyepitope was constructed with the aim of triggering an antiviral cellular immune response. It has been discovered by inventors of the present patent application that fused to the HBsAg protein, this polyepitope impairs the secretion of virus-like particles (VLPs). This invention involves the design of polynucleotides and expression vectors for cloning and expressing foreign peptides or polypeptides, such as HIV-1 polyepitopes, as tagged HBsAg fusion proteins in HBsAg VLP. Polynucleotides and expression vectors comprising these polynucleotides have been designed, all preserving recombinant HBsAg VLPs formation and secretion.

Thus, in one aspect, this invention concerns: i) the GA1xFlag-M and GA3xFlag-M polynucleotides, which once inserted in HBsAg expression vectors induce optimal expression of recombinant HBsAg VLPs; ii) new expression vectors comprising the GA1xFlag-M or GA3xFlag-M polynucleotide for optimal expression of recombinant HBsAg VLPs; iii) the use of these new expression vectors for the production of recombinant HBsAg VLPs that are secreted from host cells; and iv) the recombinant expression vectors obtained assembling the nucleic acids encoding foreign peptides or polypeptides to the new expression vectors.

More particularly, this invention provides a polynucleotide comprising GA1xFlag-M or GA3xFlag-M polynucleotide. Specifically, this invention provides a polynucleotide comprising:

[SEQ ID NO: 1]

CAGGCCATGCAGTGGAACTCCACAcccgggGCTGGAGCAGGAGCTGATTA
CAAGGACGACGACGACAAGgaattcCTGCAGGCTAGCAGATCTctcgagC
TGAACATG;

and a polynucleotide comprising:

[SEQ ID NO: 2]

CAGGCCATGCAGTGGAACTCCACAcccgggGCTGGAGCAGGAGCTGACTA
CAAAGACCACGACGGTGATTATAAAGATCACGACATTGATTACAAGGACG
ACGACGACAAGgaattcCTGCAGGCTAGCAGATCTctcgagCTGAACAT
G.

The polynucleotide can further comprise a eukaryotic promoter sequence, a nucleotide sequence encoding hepatitis B surface antigen protein (HBsAg), a polyadenylation sequence, or combinations of these elements.

In one embodiment of the invention, the polynucleotide comprises a nucleotide sequence encoding hepatitis B surface antigen protein (HBsAg) devoid of translation initiation ATG and positioned downstream and in frame with the GA1xFlag-M or the GA3xFlag-M polynucleotide sequence. The polynucleotide can comprise a polyadenylation sequence operably linked to the other sequences.

This invention provides a polynucleotide of the invention comprising the polynucleotide sequence cloned between HindIII and AvrII restriction sites in pGA1xFlag-M plasmid deposited at the CNCM on Dec. 16, 2005, under the Accession Number I-3543.

This invention also provides a polynucleotide of the invention comprising the polynucleotide cloned between HindIII and AvrII restriction sites in pGA3xFlag-M plasmid deposited at the CNCM on Dec. 16, 2005, under the Accession Number 1-3545.

In addition, this invention provides a polynucleotide hybridizing under stringent conditions to a polynucleotide of the invention, or its complement, such as variant polynucleotides resulting from degeneracy of the genetic code. Another example of a variant polynucleotide is a polynucleotide having a different restriction site or sites in the polylinker, provided that any removed or modified restriction site does not disrupt the translation frame.

Further, this invention provides a polynucleotide of the invention, which further comprises a foreign coding polynucleotide inserted in any of restriction sites of the GA1xFlag-M or GA3xFlag-M polynucleotide and in frame with the ATG at position 7 in the GA1xFlag-M or GA3xFlag-M polynucleotide sequence. The polynucleotide is useful for preparing recombinant DNA constructs prior to insertion into a vector.

A cloning and/or expression vector comprising a polynucleotide of the invention is also provided.

Further, this invention provides a eukaryotic host cell comprising a vector of the invention. In one embodiment, the vector in the eukaryotic host cell can comprise an eukaryotic promoter sequence operably linked to a nucleotide sequence encoding HBsAg protein for expression of HBsAg virus-like particles. Optionally, the vector can comprise a nucleotide sequence encoding a HBsAg fusion protein comprising a foreign polypeptide and HBsAg protein, wherein the eukaryotic host cell produces HBsAg virus-like particles constituted by the HBsAg fusion protein and HBsAg protein.

This invention also provides a method of producing HBsAg virus-like particles. The method comprises providing a host cell of the invention, and expressing the fusion proteins and HBsAg proteins under conditions in which the proteins assemble into virus-like particles, which are released from the host cell into extracellular space. Optionally, the method comprises recovering the virus-like particles.

Further, the invention provides a method of preparing a HBsAg fusion protein, wherein the method comprises providing a host cell of the invention, and expressing a tagged HBsAg fusion protein and HbsAg protein under conditions in which the proteins assemble into virus-like particles. The particles are released from the host cell into extracellular space. The VLP bearing tagged HBsAg fusion proteins from the bacteria culture can be separated by capture with Flag-M antibodies, HBsAg antibodies, or by use of both of these types of antibodies.

This invention provides an expression vector selected from the deposited, recombinant vectors pGA1xFlag-M (CNCM No.1-3543), pGA3xFlag-M (CNCM No. I-3545), pGA1xFlag-M pol.opt (CNCM No. I-3544), pGA3xFlag-M pol.opt (CNCM No. I-3546), pGA1xFlag-M.poI1A2 (CNCM No. I-3579), pGA1xFlag-M.poI2A2 (CNCM No. I-3580), pGA1xFlag-M.poI1B7 (CNCM No. I-3581), and pGA1xFlag-M.poI2B7 (CNCM No. I-3582). A preferred embodiment of this invention provides a polynucleotide comprising the sequence between HindIII and AvrII restriction sites of one of these expression vectors.

This invention provides a polypeptide encoded by a polynucleotide or by a vector according to the invention.

### BRIEF DESCRIPTION OF THE DRAWINGS

This invention will be described with reference to the drawings in which:

FIG. 1(A) is a schematic representation of plasmid pCMV-B10.

FIG. 1(B) is the complete nucleic acid sequence of pCMV-basic.

FIG. 2 is the nucleic acid sequence of pGA1xFlag-M.

FIG. 3 is the nucleic acid sequence of pGA3xFlag-M.

FIG. 4 is a more detailed nucleic acid sequence for the GA1xFlag-MpoI.opt insert in pGA1xFlag-M to produce pGA1xFlag-MpoI.opt.

FIG. 5 is a more detailed nucleic acid sequence for the GA3xFlag-MpoI.opt insert in pGA3xFlag-M to produce pGA3xFlag-MpoI.opt.

FIG. 6 is the nucleic acid sequence of pGA1xFlag-MpoI.opt.

FIG. 7 is the nucleic acid sequence of pGA3xFlag-MpoI.opt.

FIG. 8 depicts the secretion kinetics corresponding to pGA1xFlag-M, pGA1xFlag-MpoI.opt, pGA3xFlag-M and pGA3xFlag-MpoI.opt.

FIG. 9 depicts the results of a semi-quantitative anti-Flag-M ELISA on transfected SW480 supernatants.

FIG. 10 is pGA1xFlag-M.poI1A2 nucleic acid sequence (in bold: poI1A2 polyepitope).

FIG. 11 is pGA1xFlag-M.poI2A2 nucleic acid sequence (in bold: poI2A2 polyepitope).

FIG. 12 is pGA3xFlag-M.poI1A2 nucleic acid sequence (in bold: poI1A2 polyepitope).

FIG. 13 is pGA3xFlag-M.poI2A2 nucleic acid sequence (in bold: poI2A2 polyepitope).

FIG. 14 is pGA1xFlag-M.poI1B7 nucleic acid sequence (in bold: poI1B7 polyepitope).

FIG. 15 is pGA1xFlag-M.poI2B7 nucleic acid sequence (in bold: poI2B7 polyepitope).

FIG. 16 is pGA3xFlag-M.poI1B7 nucleic acid sequence (in bold: poI1B7 polyepitope).

FIG. 17 is pGA3xFlag-M.poI2B7 nucleic acid sequence (in bold: poI2B7 polyepitope).

FIG. 18 depicts the secretion kinetics corresponding to pGA1xFlag-MpoI1.A2 and pGA1xFlag-MpoI2.A2.

FIG. 19 depicts the secretion kinetics corresponding to pGA3xFlag-MpoI1.A2 and pGA3xFlag-MpoI2.A2.

FIG. 20 depicts the secretion kinetics corresponding to pGA1xFlag-MpoI1.B7 and pGA1xFlag-MpoI2.B7.

FIG. 21 depicts the secretion kinetics corresponding to pGA3xFlag-MpoI1.B7 and pGA3xFlag-MpoI2.B7.

### DETAILED DESCRIPTION OF THE INVENTION

The hepatitis B surface antigen (HBsAg) can assemble into sub-virion virus like particles (VLPs). By fusing immunogenic peptides to the amino-terminus of HBsAg, several bivalent vaccines have been developed. Notably, a polyepitope bearing HIV-1 epitopes restricted to the HLA-A*0201 class I allele elicited a significant HIV-1 specific CD8⁺ cytotoxic T lymphocyte (CTL) response in vivo (12). This recombinant HBsAg failed to form VLPs due to retention in the Golgi apparatus.

In contrast, this invention provides polynucleotides and expression vectors for the production of recombinant proteins as tagged HBsAg fusion proteins, which assemble into VLPs and which are efficiently secreted by host cells. It is thus possible to make self-assembling recombinant HBsAg VLPs with residues of another protein. This is demonstrated for HIV-1 polyepitopes, and thus provides efficient bivalent HBV/HIV vaccines, which are particularly apposite given that these two viruses are frequently associated.

More particularly, this invention provides two polynucleotide motifs (GA1xFlag-M and GA3xFlag-M) for cloning and expressing foreign sequences fused in frame to HBsAg. In a particular embodiment of the present patent application, these polynucleotides have been inserted into two distinct expression vectors designated herein as pGA1xFlag-M and pGA3xFlag-M. These plasmids were constructed as follows.

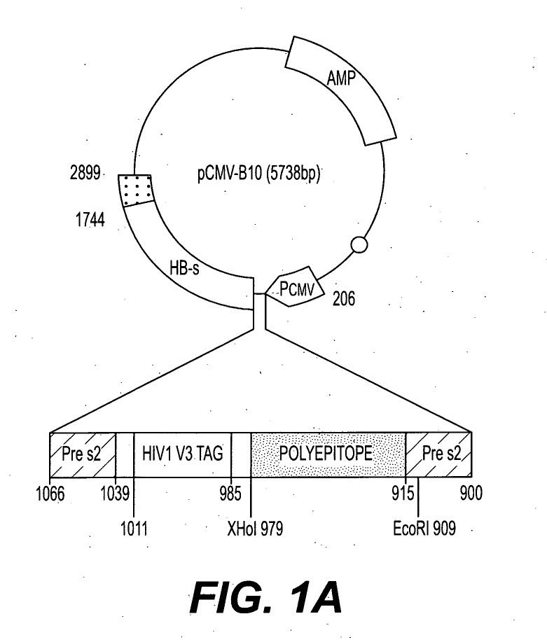

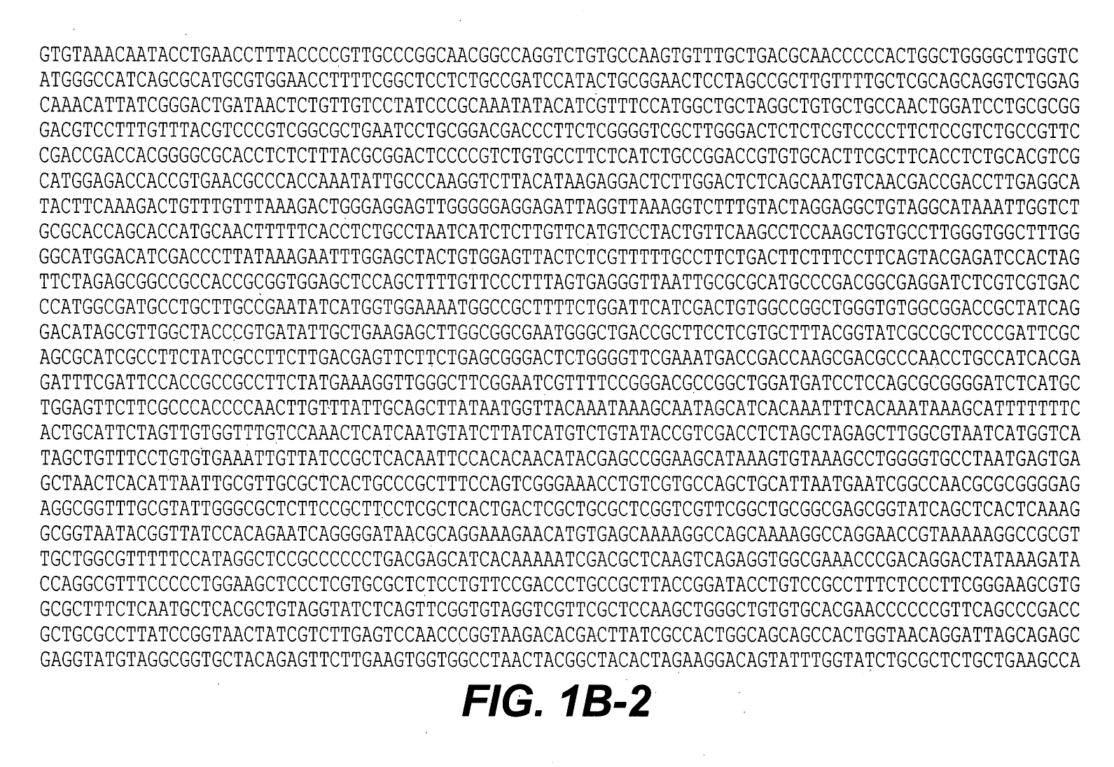

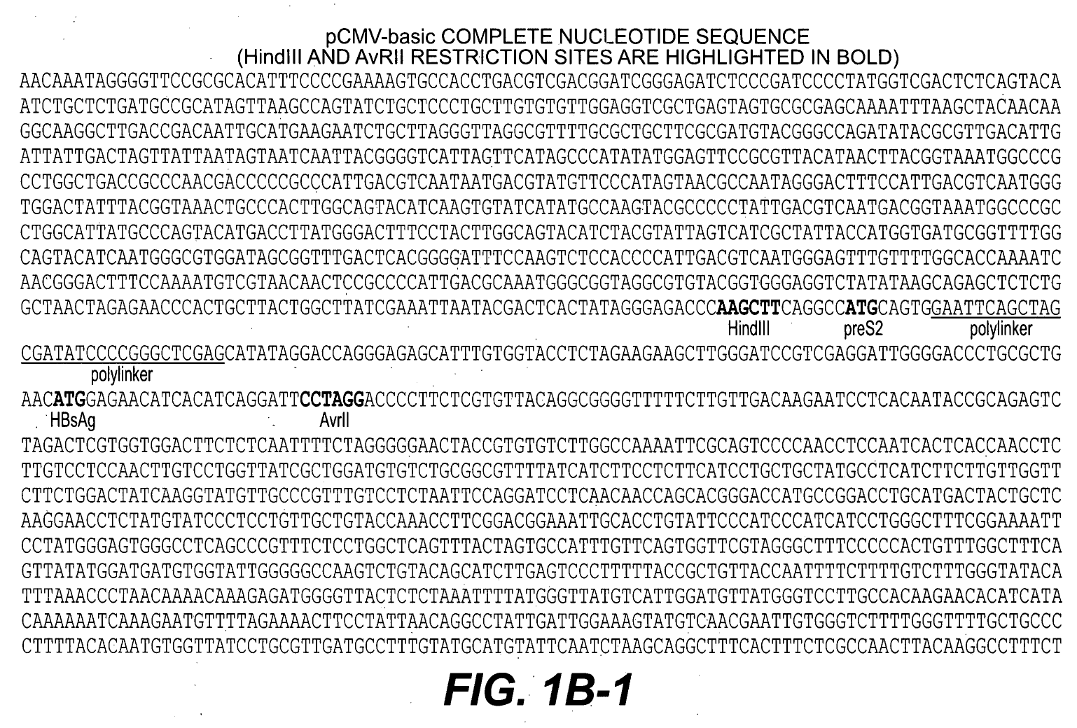

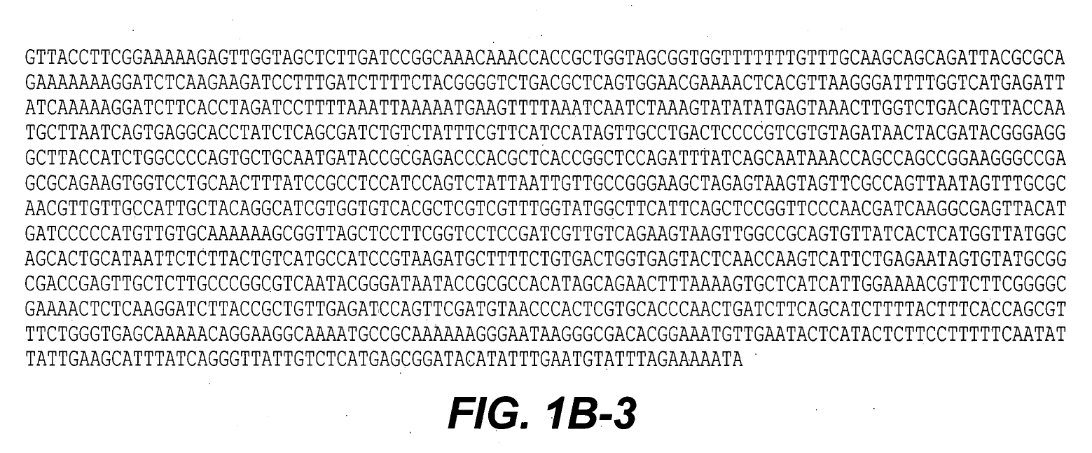

The plasmid pCMV-B10 was previously known. See Eur. J. Immunol. 2001, 31:3064-3074. FIG. 1 (A) is a schematic representation of the pCMV-B10 plasmid.

Specifically, the pCMV-B1 plasmid vector is a pcDNA3 derivative (Invitrogen, Costa Mesa, Calif.), in which the nucleotide sequences of the hepatitis B middle [initiation at Position 900 (ATG), termination at Pos. 1744 (TAA)] and small [initiation at Pos. 1066 (ATG), termination at Pos. 1744 (TAA)] envelope proteins have been inserted downstream of a human CMV immediate early promoter. A polyadenylation signal is provided by the HBV untranslated sequence (nucleotides 1744-2899). The central part of the coding preS2 segment sequence was replaced by a polylinker in which a polyepitope DNA can be inserted. An HIV-1 derived (MN isolate) V3 loop tag was inserted, downstream of the polyepitope.

Using the pCMV-B1 plasmid, a plasmid designated ppoIHIV-1.opt was constructed by cloning an HIV-1 polyepitopic sequence between the EcoRI and XhoI restriction sites in pCMV-B10. The HIV-1 polyepitopic sequence is identified as poIHIV-1.opt and has the following amino acid sequence:

YLKEPVHGVRAKTYLNAWVKVVRDTAVLDVGDAYFSVRAKTYLVKLWYQL
RADTRLYNTVATLRTKALLDTGADDTVRAKTLLWKGEGAVRTDAYIYQYM
DDLR.

Using the ppoIHIV-1.opt plasmid, a control plasmid, pCMV-basic (FIG. 1B), was constructed for use in expression studies involving the vectors of the invention. Specifically, the poIHIV-1.opt polyepitope in ppoIHIV-1.opt was removed by digestion with EcoRI and XhoI and substituted by a polylinker comprising EcoRI, NheI, EcoRV, SmaI, and XhoI restriction sites. In between the EcoRI and XhoI restriction sites, the NheI, EcoRV, SmaI restriction sites follow one another as shown below.

### Polylinker of the pCMV-basic Plasmid (EcoRI-XhoI) Nucleic Acid Sequence

GAATTC-A-GCTAGC-GATATC-CCCGGG-CTCGAG

### Restriction Enzyme Sequence

EcoRI-NheI-EcoRV-SmaI-XhoI.

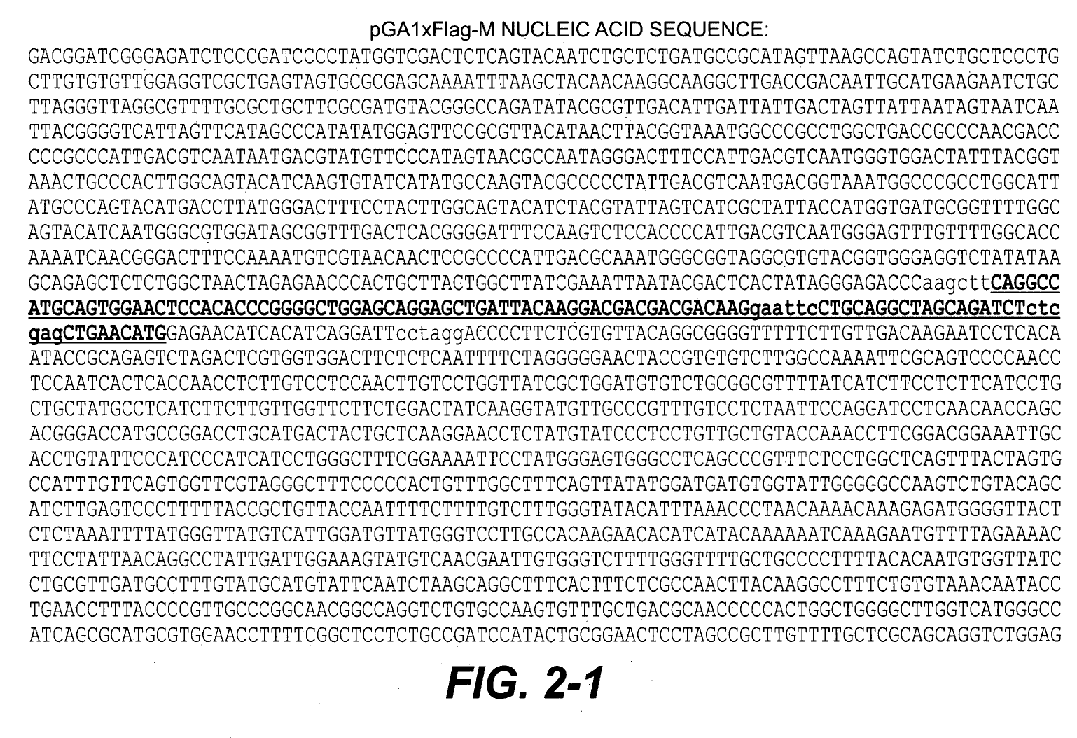

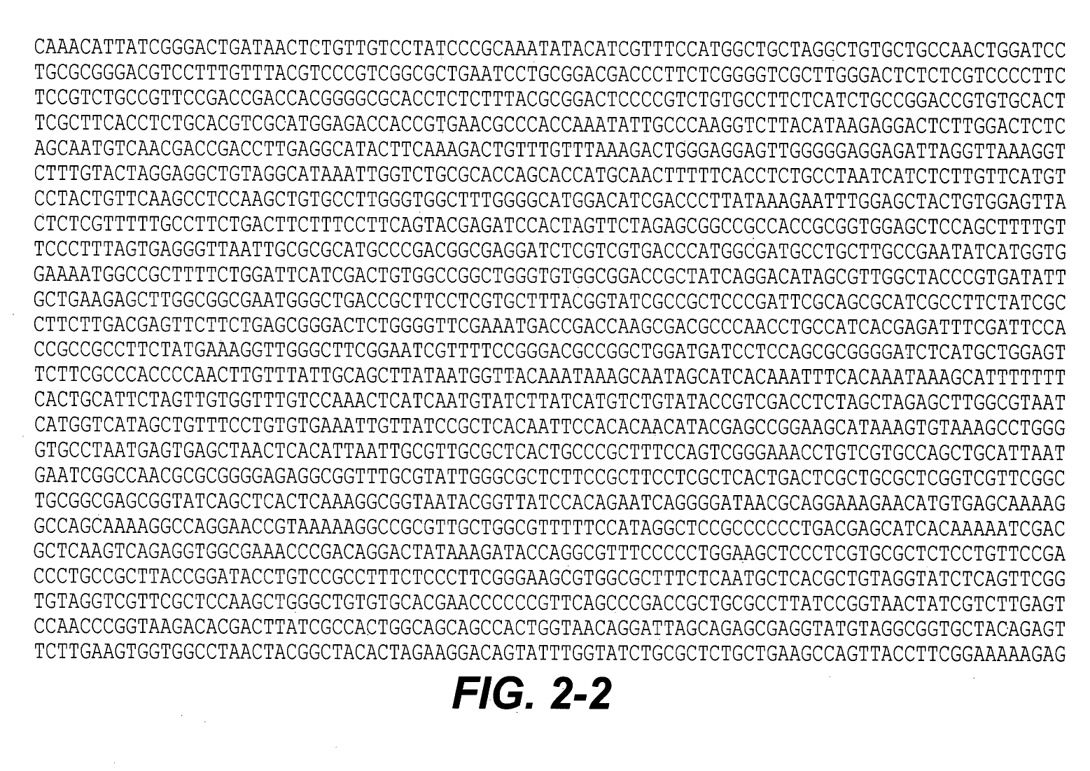

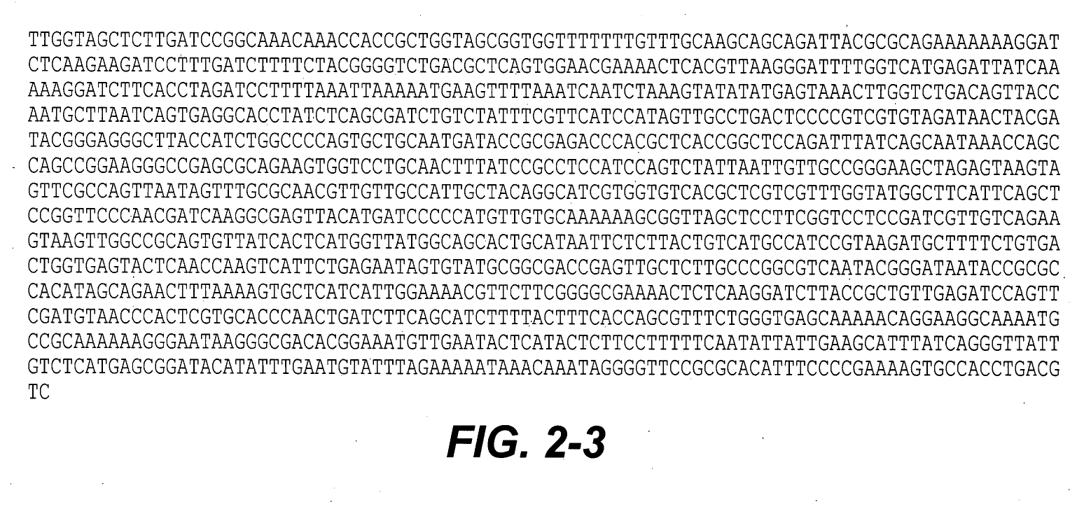

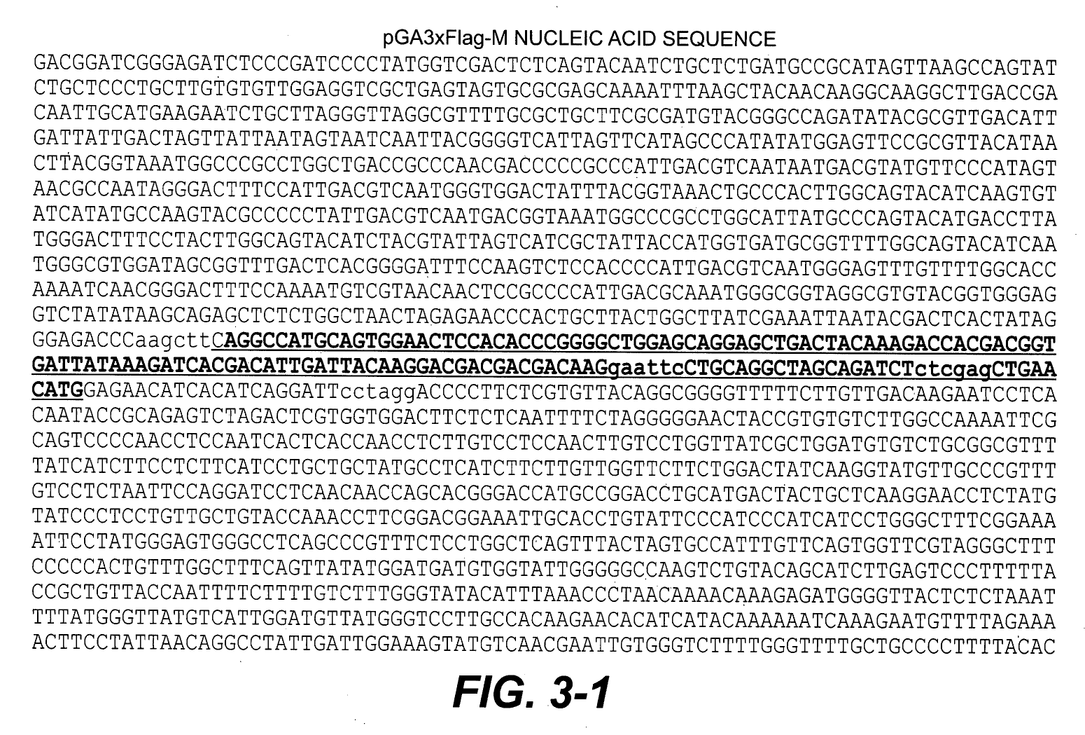

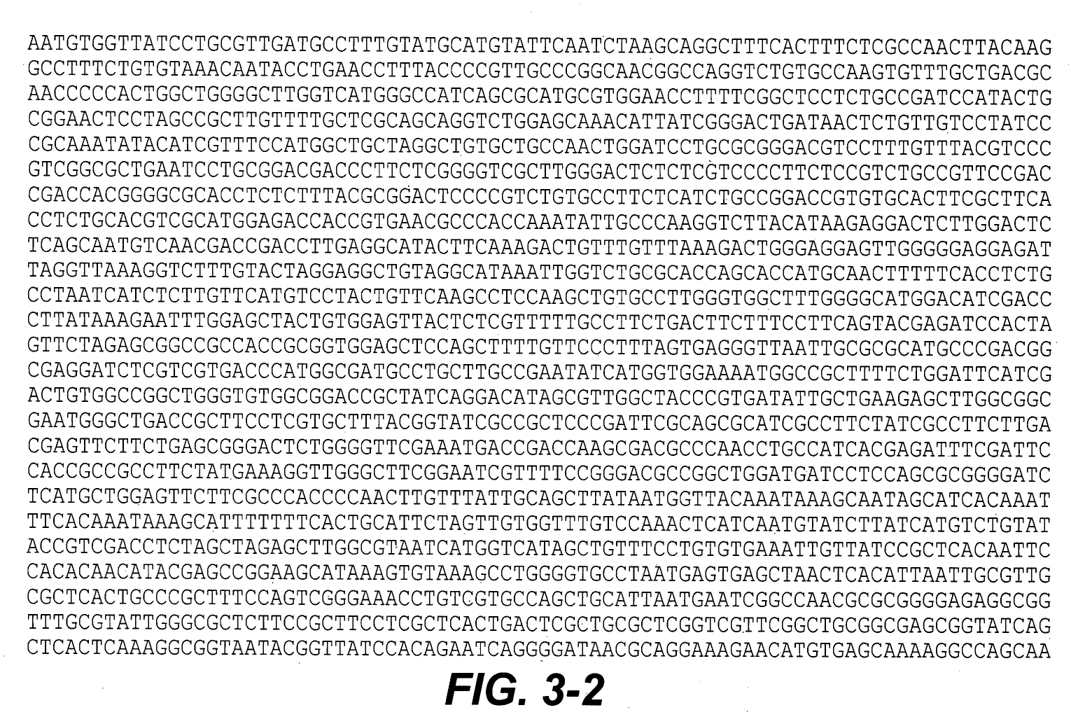

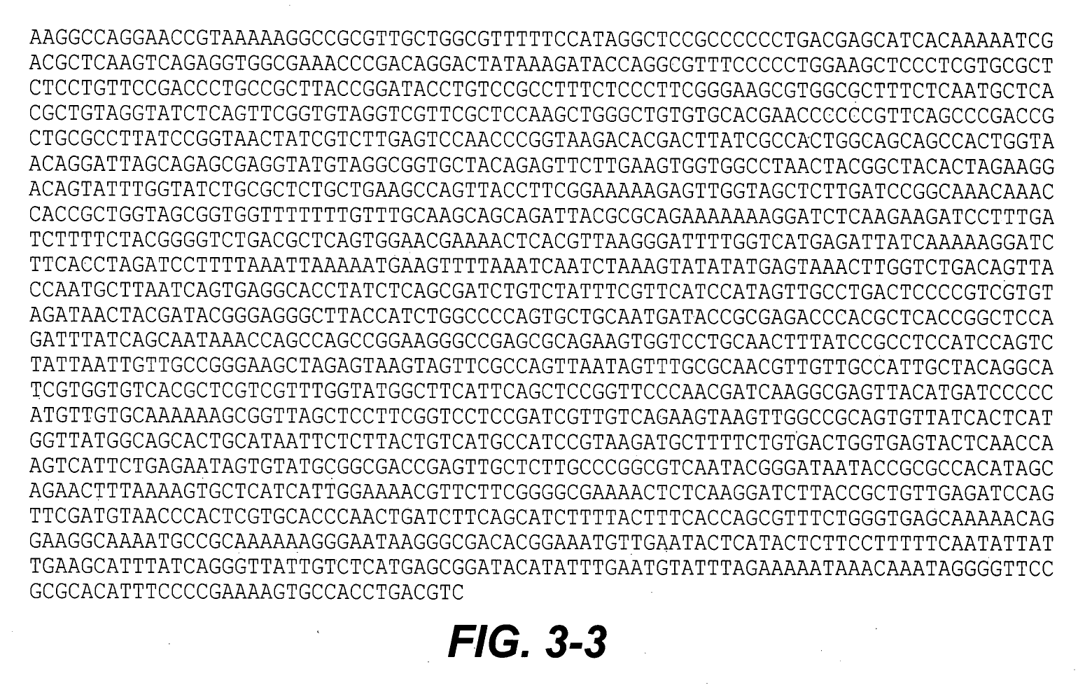

The pGA1xFlag-M (FIG. 2) and pGA3xFlag-M (FIG. 3) plasmids of the invention were then constructed from the pCMV-basic plasmid. Specifically, the newly created pGA1xFlag-M and pGA3xFlag-M constructions only maintained the pCMV-basic plasmid backbone, with its CMV promoter and HBV polyadenylation signal. The nucleic acid sequence between the HindIII and the AvrII restriction sites in the pCMV-basic plasmid was eliminated. Nucleic acid sequences were then cloned between the HindIII and AvrII restriction sites. Nucleic acid sequences corresponding to the cloned sequences in between the HindIII and AvrII of the pCMV-basic plasmid are given below for the pGA1xFlag-M and pGA3xFlag-M plasmids:

pGA1xFlag-M and pGA3xFlag-M Nucleic Acids Sequences Between the HindIII (aagctt) and AvrII (cctagg) Restrictions Sites
Nucleic Acid Sequence of pGA1xFlag-M

aagcttCAGGCCATGCAGTGGAACTCCACACCCGGGGCTGGAGCAGGAGC
TGATTACAAGGACGACGACGACAAGgaattcCTGCAGGCTAGCAGATCTc
tcgagCTGAACATGGAGAACATCACATCAGGATTcctagg

Nucleic Acid Sequence of pGA3xFlag-M

aagcttCAGGCCATGCAGTGGAACTCCACACCCGGGGCTGGAGCAGGAGC
TGACTACAAAGACCACGACGGTGATTATAAAGATCACGACATTGATTACA
AGGACGACGACGACAAGgaattcCTGCAGGCTAGCAGATCTctcgagCTG
AACATGGAGAACATCACATCAGGATTcctagg

EcoRI (gaattc) and XhoI (ctcgag) restriction sites are provided in these sequences for cloning foreign sequences in the vectors.

More particularly, starting from the HindIII restriction site in 5′, nucleotides encoding the QA peptide corresponding to C-terminal sequence of the HBV preS1 region were introduced. These nucleotides were inserted to preserve the native context of ATG start codon of preS2, hence the strength of this translation initiation codon. Then nucleotides encoding the MQWNSTP peptide corresponding to the N-terminal portion of the HBV preS2 region were introduced. This modification was made to reintroduce in the pGA1xFlag-M and pGA3xFlag-M plasmids the glycosylation site (N4*), which was absent in the pCMV-basic construction.

The preS2 peptide is followed by the GA motif (Gly-Ala amino acids repeated three times), here located to prevent steric impairment for the binding of anti-Flag antibodies to the tag (1xFlag-M or 3xFlag-M) by sugar molecules covalently linked to preS2 N4*.

The GA motif is followed by the 1xFlag-M or 3xFlag-M tag (modified from SIGMA-ALDRICH), giving the pGA1xFlag-M and pGA3xFlag-M plasmids, respectively. Specifically, the 1xFlag and 3xFlag nucleic acid sequences from SIGMA-ALDRICH were modified to eliminate the ATG codons in the second and third possible reading frames of the sequences. Nucleic acid sequences of the original SIGMA-ALDRICH1xFlag and 3xFlag were modified preserving amino acids sequences, but eliminating methionine residues in secondary and tertiary phases.

In the pGA1xFlag-M and pGA3xFlag-M plasmids, between the EcoRI and XhoI restriction sites, a new polylinker was inserted, where the PstI, NheI, BgIII restriction sites follow one the others.

In the pGA1xFlag-M and pGA3xFlag-M plasmids, the preS2 C-terminal peptide preceding the HBsAg ATG start codon (M₁) was reduced to two amino acids (Leu-Asn), preserving the nucleic acid context, hence the strength, of the HBsAg translation initiation ATG codon. Finally, nucleic acid sequence of HBsAg protein from the ATG start codon to the seventh codon was inserted in the pGA1xFlag-M and pGA3xFlag-M plasmids in order to preserve the N-terminal sequence of HBsAg deleted by HindIII-AvrII digestion.

The sequence cloned between HindIII and AvrII sites in pGA1xFlag-M and pGA3xFlag-M plasmids were obtained by “atypical” PCR, as described below, using long primers (60-80 nucleic acids) all corresponding to the 5′-3′ strand of the final product, and cloning in between the HindIII and AvrII of the pCMV-basic plasmid.

The nucleic acid sequences of pGA1xFlag-M and pGA3xFlag-M are given in FIGS. 2 and 3, respectively. In FIG. 2, the GA1xFlag-M sequence is underlined. The GA1xFlag-M nucleic acid sequence in bold corresponds to the following amino acid sequence, which represents in the order: the preS1 C-terminal sequence (QA)-the preS2 ATG-the glycosylation site (N* in the N-terminal protion of preS2 polypeptide QWNSTP)-the GA motif-the 1xFlag-M tag-the polylinker-the preS2 C-terminal portion (LN)-the HBsAg ATG:

QAMQWN*STPGAGAGADYKDDDDKEFLQASRSLELNM.

The 1xFlag-M sequence is underlined above. The remainder of the sequence depicted is the vector.

In FIG. 3, the GA3xFlag-M sequence is underlined. The GA3xFlag-M nucleic acid sequence in bold corresponds to the following amino acid sequence which represents in the order: the preS1 C-terminal sequence (QA)-the preS2 ATG-the glycosylation site (N* in the N-terminal protion of preS2 polypeptide QWNSTP)-the GA motif-the 3xFlag-M tag-the polylinker-the preS2 C-terminal portion (LN)-the HBsAg ATG:

QAMQWN*STPGAGAGADYKDHDGDYKDHDIDYKDDDDKEFLQASRSLELN
M.

The 3xFlag-M sequence is underlined above.

The polynucleotides and plasmids of the invention are particularly useful for cloning and expressing foreign sequences. Polynucleotides and plasmids provide the nucleic sequence context to produce a fusion polypeptide where the HBsAg protein is the carrier, referred herein as HBsAg fusion protein, which is efficiently secreted by host cells and assembles with HBsAg protein as HBsAg virus-like particles. HBsAg fusion proteins produced by using the polynucleotides of the invention and the expression vectors of the invention have the relevant characteristic to carry the 1xFlag or 3xFlag tag. For this reason, HBsAg fusion proteins of the invention are also named tagged HBsAg fusion proteins.

The HBsAg fusion proteins are characterized herein as “tagged” because of the presence of 1xFlag-M and 3xFlag-M tags in the GA1xFlag-M and GA3xFlag-M motifs, respectively. This tag is very relevant because 1) it makes it possible to follow the incorporation of HBsAg fusion proteins in VLP; 2) it can be used to purify recombinant VLPs from the cell culture medium; and 3) it can be used to further purify or isolate the HBsAg fusion protein from the VLP and from the HBsAg proteins of the recombinant VLP.

It is interesting to note that even without foreign nucleotide sequence insertion, two types of proteins are synthesized by the expression vectors of the invention as pGA1xFlag-M and pGA3xFlag-M : the HBsAg protein and a tagged HBsAg protein translated from the preS2 ATG and carrying the Flag tag. The tagged HBsAg VLP obtained by assembling these two proteins makes it possible to follow the formation of the VLP in host cells and to study the kinetics of secretion of HBsAg VLP.

The term “peptide” is generally understood in the art to refer to a small amino acid molecule, whereas the term “polypeptide” is generally understood to refer to a larger amino acid molecule. Both peptides and polypeptides are within the scope of this invention. Thus, for example, the foreign sequence can be either a peptide or a polypeptide. The terms are used interchangeably herein.

The HBsAg proteins and fusion polypeptides can assemble with host cell derived lipids into multimeric particles that are highly immunogenic in comparatively low concentrations. The HBsAg fusion proteins containing the foreign sequence are exposed on the surface of the virus-like particles. The resulting virus-like particles provide excellent configurational mimics for protective epitopes as they exist in pathogens, such as an infectious virus. For these reasons, the virus-like particles are suitable for exploitation as carriers for foreign peptides or polypeptides, such as protective determinants of etiologic agents. The foreign peptides and polypeptides are comprised of sequences other than HBsAg sequences. These highly immunogenic virus-like particles display epitopes of the foreign peptides or polypeptides while retaining the protective response to HBsAg determinants.

Fusion proteins containing a foreign peptide or polypeptide and a very small part of preS2 region and HBsAg protein are alternatively referred to herein as HBsAg fusion protein or the recombinant HBsAg fusion protein.

The HBsAg virus-like particles thus comprise a mixture of the HBsAg proteins and fusion polypeptides comprising the foreign peptide inserted in the preS2 part of M protein. In one embodiment of the invention, the foreign peptide or polypeptide is a peptide or polypeptide other than a peptide or polypeptide from HBsAg.

The foreign peptide or polypeptide can contain from 8-11 to 138-140 amino acid residues, preferably from about 20-26 to about 138-140 amino acid residues, especially from about 63-64 to about 138-140 amino acid residues. Preferably, the foreign peptide or polypeptide inserted in the preS2 region is free of cysteine residues and contains 0 to 1 methionine residues apart from the methionine required for initiation of preS2 translation. Flanking residues on either the N-terminal, C-terminal, or both N- and C-terminal ends may be added to the foreign peptide or polypeptide to generate the virus-like particles. In one aspect, the invention provides virus-like particles comprising epitope-bearing portions of foreign peptides or polypeptides. These epitopes are immunogenic or antigenic epitopes of the foreign peptides or polypeptides. An “immunogenic epitope” is defined as a part of a protein that elicits a humoral or cellular response in vivo when the whole polypeptide or fragment thereof, is the immunogen. A region of a polypeptide to which an antibody can bind is defined as an “antigenic determinant” or “antigenic epitope.” Included in the present invention are VLPs containing both immunogenic epitopes and antigenic epitopes. Foreign peptides or polypeptides comprising immunogenic or antigenic epitopes are at least 7 amino acids residues in length.

The foreign peptide or polypeptide can also be derived from any number of foreign proteins, i.e. proteins other than the envelope proteins of HBV. The foreign peptide or polypeptide can be derived from any protein of any plant, animal, bacterial, viral or parasitic organism. In one embodiment the foreign peptide or polypeptide can be derived from a polypeptide of a pathogen The term “pathogen” as used herein, means a specific causative agent of disease, and may include, for example, any bacteria, virus, or parasite. The term “disease” as used herein, means an interruption, cessation, or disorder of body function, system, or organ. Typical diseases include infectious diseases. For example, the foreign peptide or polypeptide can be from the immunogenic proteins of an RNA virus, such as HIV-1, HIV-2, SIV, HCV, Ebola virus, Marbourg virus, HTLV-I, and HTLV-II. Specific examples are the structural or NS1 proteins of Dengue virus; the G1, G2, or N proteins of Hantaan virus; the HA proteins of Influenza A virus; the Env proteins of Friend murine leukemia virus; the Env proteins of HTLV-1 virus; the preM, E, NS1, or NS2A proteins of Japanese encephalitis virus; the N or G proteins of Lassa virus; the G or NP proteins of lymphocytic choriomeningitis virus; the HA or F proteins of measles virus; the F or HN proteins of parainfluenza 3 virus; the F or HN proteins of parainfluenza SV5 virus; the G proteins of Rabies virus; the F or G proteins of respiratory syncytial virus; the HA or F proteins of Rinderpest; or the G proteins of vesicular stomatitis virus. These are just some of the possibilities and do not represent an exhaustive or restricted list.

The foreign peptide or polypeptide can also be derived from the immunogenic proteins of a DNA virus, such as gp89 of cytomegalvirus; gp340 of Epstein-Barr; gp13 or 14 of equine herpesvirus; gB of herpes simplex 1; gD of Herpes simplex 1; gD of herpes simplex 2; or gp50 of pseudorabies. These are just some of the possibilities and do not represent an exhaustive or restricted list.

Further, the foreign peptide or polypeptide can be derived from the immunogenic proteins of bacteria, such as Streptococci A M6 antigens, or tumor antigens, such as human melanoma p97, rat Neu oncogene p185, human epithelial tumor ETA, or human papillomarvirus antigens. These are just some of the possibilities and do not represent an exhaustive or restricted list.

In a preferred embodiment of this invention, the foreign peptide or polypeptide is derived from a human immunodeficiency virus. Following are HIV-1 epitopes that can be employed in designing the foreign peptide or polypeptide.

| GAG | P17 (77-85) | SLYNTVATL (S9L) |
|---|---|---|
| | P24(19-27) | TLNAWVKW (T9V) |
| POL | (79-88) | LLDTGADDTV (L10V) |
| | (263-273) | VLDVGDAYFSV (V11V) |
| | (334-342) | VIYQYMDDL (V9L) |
| | (464-472) | ILKEPVHGV (19V) |
| | (576-584) | PLVKLWYQL (P9L) |
| | (669-679) | ESELVNQIIEQ (E11Q) |
| | (671-680) | ELVNQIIEQL (E10 |
| | (956-964) | LLWKGEGAV (L9V) |
| ENV | Gp41 (260-268) | RLRDLLLIV (R9V) |
| NEF | (188-196) | AFHHVAREL (A9L) |

Numbering is based on the amino acid sequence of the HIV-1 WEAU clone 1.60 (Genbank accession no. U21135). The WEAU sequence may not be always identical to that of the reactive peptide and simply indicates its location in the viral proteins.

The foreign peptide or polypeptide can comprise a multiplicity of epitopes linked to each other. It will be understood that the virus-like particles of the invention can contain multiple epitopes of one or more origins, such as epitopes from different immunogenic proteins of the same pathogen. It will also be understood that the virus-like particles can contain one or more epitopes from different pathogens. In addition, mixtures of virus-like particles having different epitopes in different particles are contemplated by this invention.

Recombinant expression vectors containing a nucleic acid encoding the foreign peptide or polypeptide in VLPs can be prepared using well known methods. The expression vectors include the sequence encoding the foreign peptide or polypeptide operably linked to suitable transcriptional or translational regulatory nucleotide sequences, such as those derived from a mammalian, viral, or insect gene. A transcriptional or translational regulatory nucleotide sequence is operably linked if the nucleotide sequence controls the transcription or translation of another coding DNA sequence. Examples of regulatory sequences include transcriptional promoters, operators, or enhancers, an mRNA ribosomal binding site, and appropriate sequences which control transcription and translation initiation or termination. The ability to replicate in the desired host cells, usually conferred by an origin of replication, and a selection gene by which transformants are identified can additionally be incorporated into the expression vector.

Among eukaryotic vectors for use in the preparation of vectors of the invention are pWLNEO, pSV2CAT, pOG44, pXT1, and pSG available from Stratagene; and pSVK3, PBPV, pMSG, and pSVL available from Pharmacia. Other suitable vectors will be readily apparent to the skilled artisan.

Among vectors for use in the preparation of vector of the invention, non-integrative eucaryotic vectors are not only useful, but integrative/transformant vectors (i.e. vectors that integrate a part of their nucleic acid material in the genome of the eukaryotic host cell) can also be employed. Typical of these vectors are lentiviral vector Trips, adenovirus, and yeast integrative vectors.

The expression vectors of the invention can include at least one selectable marker. Such markers include, for example, dihydrofolate reductase, G418, ampicilin or neomycin resistance for eukaryotic cell culture.

Any strong promoter known to those skilled in the art can be used for driving expression. Suitable promoters include adenoviral promoters, such as the adenoviral major late promoter; heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the β-actin promoter; and human growth hormone promoters. The promoter also can be a native promoter from HBV.

Suitable host cells for expression of VLP include yeast or higher eukaryotic cells. Appropriate cloning and expression vectors for use with plant, fungal, yeast, and mammalian cellular hosts are described, for example, in Pouwels et al. Cloning Vectors: A Laboratory Manual, Elsevier, New York, (1985). Representative examples of appropriate hosts include, but are not limited to, fungal cells, such as yeast cells; insect cells, such as Drosophila S2 and Spodoptera Sf9 cells; animal cells, such as CHO, COS, 293, and Bowes melanoma cells; and plant cells. Appropriate culture mediums and conditions for the above-described host cells are known in the art.

Introduction of the vector of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986).

In another aspect, the invention is directed to a method for producing, in vitro, HBsAg virus-like particles, comprising: culturing in vitro, in a suitable culture medium, a cell incorporating an expression vector of the invention or a polynucleotide of the invention, and collecting in the culture medium HBsAg virus-like particles produced by these cells. The virus-like particles are released form the host cell into the extracellular space.

Therefore, the invention is also concerned with cells, such as recombinant eucaryotic cells, infected, transformed, or transfected by a polynucleotide or vector of the invention for expressing the HBsAg virus-like particles. Methods for producing such cells and methods for using these cells in the production of proteins or peptides are well known in the art. The virus-like particles can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography.

While this invention relates to HBsAg virus-like particles carrying one or more polyepitopes of foreign peptides or polypeptides on their surfaces, this invention contemplates the use of polyepitopes that have been optimized for incorporation in virus-like particles. The polyepitope nucleic and amino acid sequences can be optimized in view of increasing the oveall hycrophilicity ofpolyepitope and ensuring an optimal processing of epitopes. Epitopes in the polyepitope can be permutated in order to obtain the best hydrophilic profile. Hydrophilic spacers can be added to counterbalance the generally hydrophobic class I epitopes. Epitopes bearing cysteine residues can be eliminated, the number of internal methionine residues can be limited to a minimum, and optionally Homo sapiens codon usage can be adopted. Epitopes can be positioned in head-tot-tail and an arginine residue can be inserted at the epitope Cl terminal position. As an example, the surface antigen (HBsAg) of the Hepatitis B virus (HBV) carries all the information required for membrane translocation, particle assembly, and secretion from mammalian cells. HBsAg assembles into VLPs polymeric structure that enhances antigenic stability. It is only if assembled in VLPs that HBsAg can be secreted out of cells. In this system, secretion provides high-density HBsAg presentation to antigen presenting cells (APCs). It is preferable to utilize criteria for optimizing the polyepitope sequence, which ensure the conservation of recombinant virus-like particle structures and secretion, once the particle is used as carrier of a polyepitope. Procedures for optimizing epitopes for incorporation in VLPs are described in detail in the U.S. Provisional application filed concurrently herewith by the same inventors and entitled RECOMBINANT HBsAg VIRUS-LIKE PARTICLES CONTAINING POLYEPITOPES OF INTEREST, THEIR PRODUCTION AND USE, attorney docket No. 03495.6115, the entire disclosure of which is relied upon and incorporated by reference herein.

Several polyepitopic sequences of HIV-1 were prepared for incorporation in the vectors of the invention and HBsAg virus-like particles were produced and assayed for activity. One such polyepitopic sequence was derived from epitopes of HIV-1 and has been designated poIHIV-1.opt. Following is the poIHIV-1.opt polyepitope amino acid sequence: polHIV-1 .opt

### Nucleic Acid Sequence

CTACTTGAAAGAGCCAGTTCATGGGGTGAGAGCCAAGACCTACCTGAATG
CATGGGTGAAAGTTGTCAGAGACACCGCAGTGCTGGATGTGGGGGATGCC
TACTTCTCAGTGAGAGCTAAGACTTATCTGGTCAAACTCTGGTACCAGTT
GAGGGCTGACACTCGTCTTTACAACACTGTGGCCACCCTTAGGACCAAGG
CTCTTCTGGACACTGGAGCAGATGACACTGTGAGGGCTAAGACCCTGCTG
TGGAAGGGAGAGGGAGCAGTTAGGACTGATGCTTACATCTACCAGTATAT
GGATGACCTTAGA

### Amino Acid Sequence

YLKEPVHGVRAKTYLNAWVKVVRDTAVLDVGDAYFSVRAKTYLVKLWYQL
RADTRLYNTVATLRTKALLDTGADDTVRAKTLLWKGEGAVRTDAYIYQYM
DDLR.

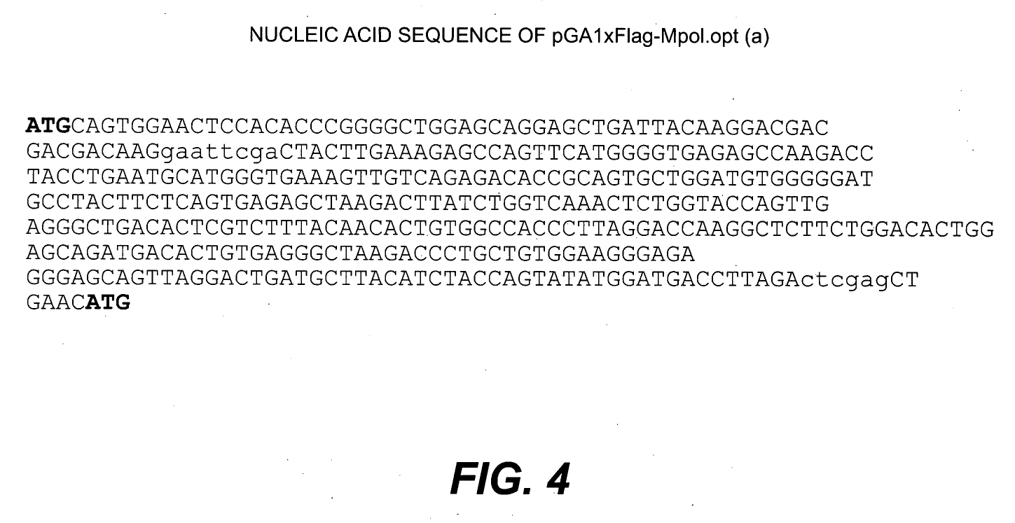

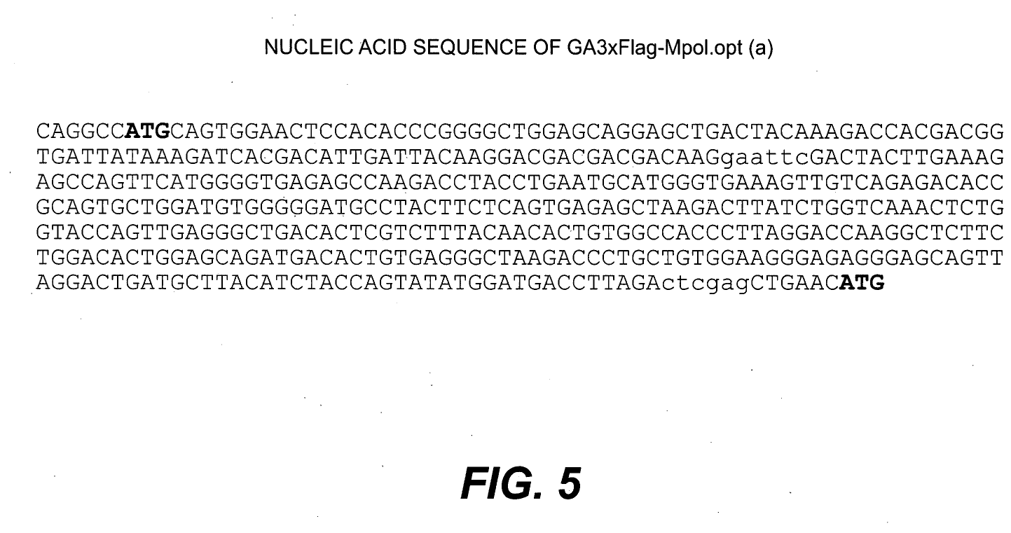

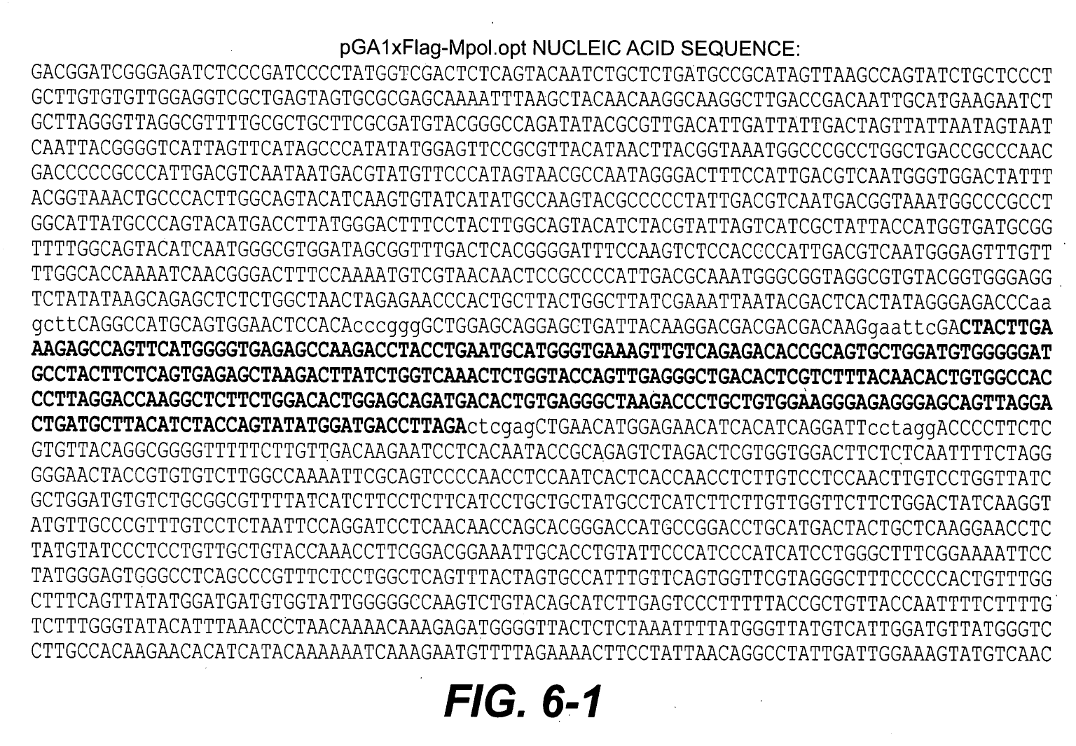

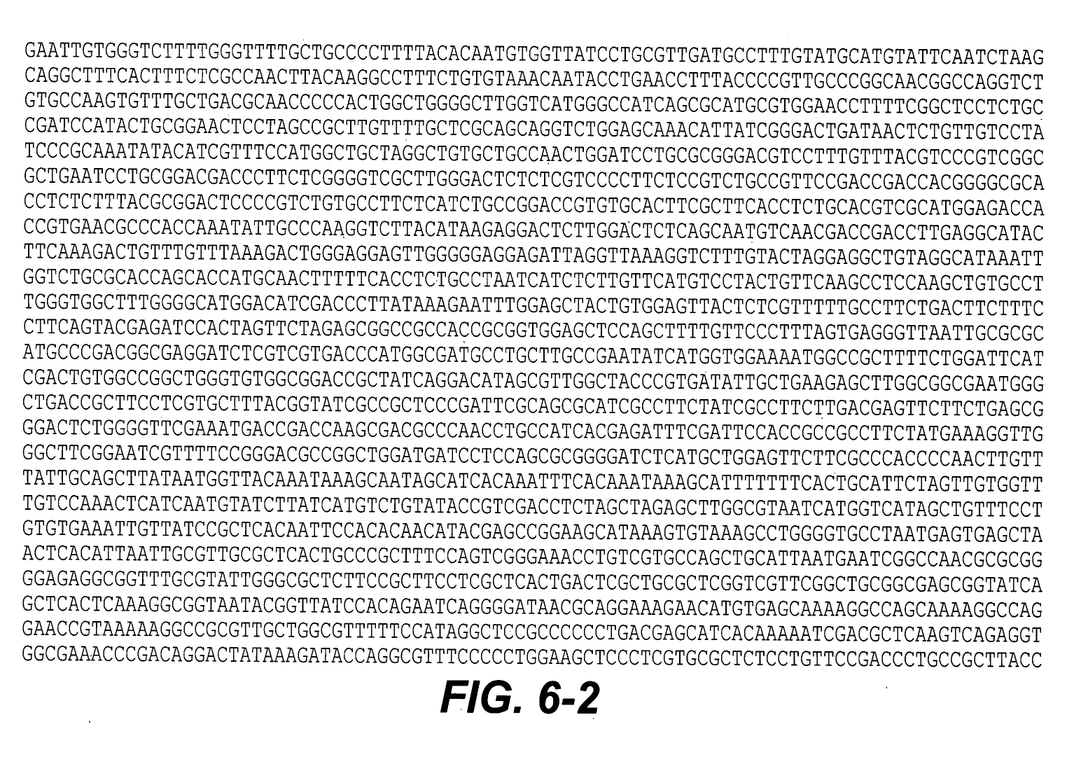

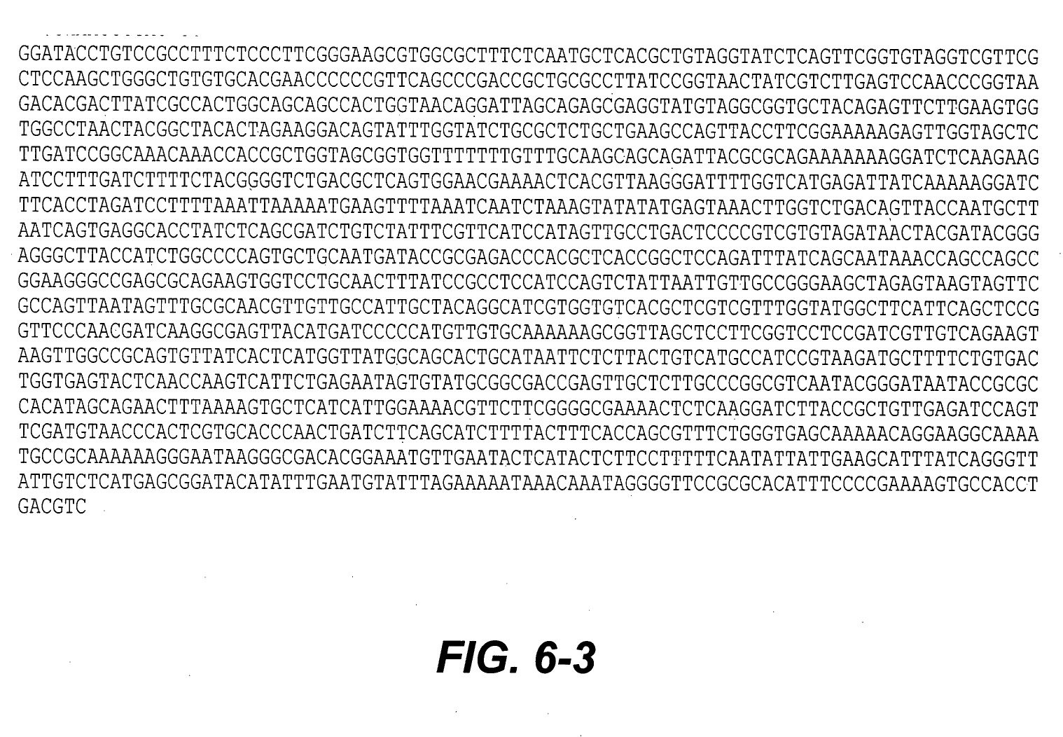

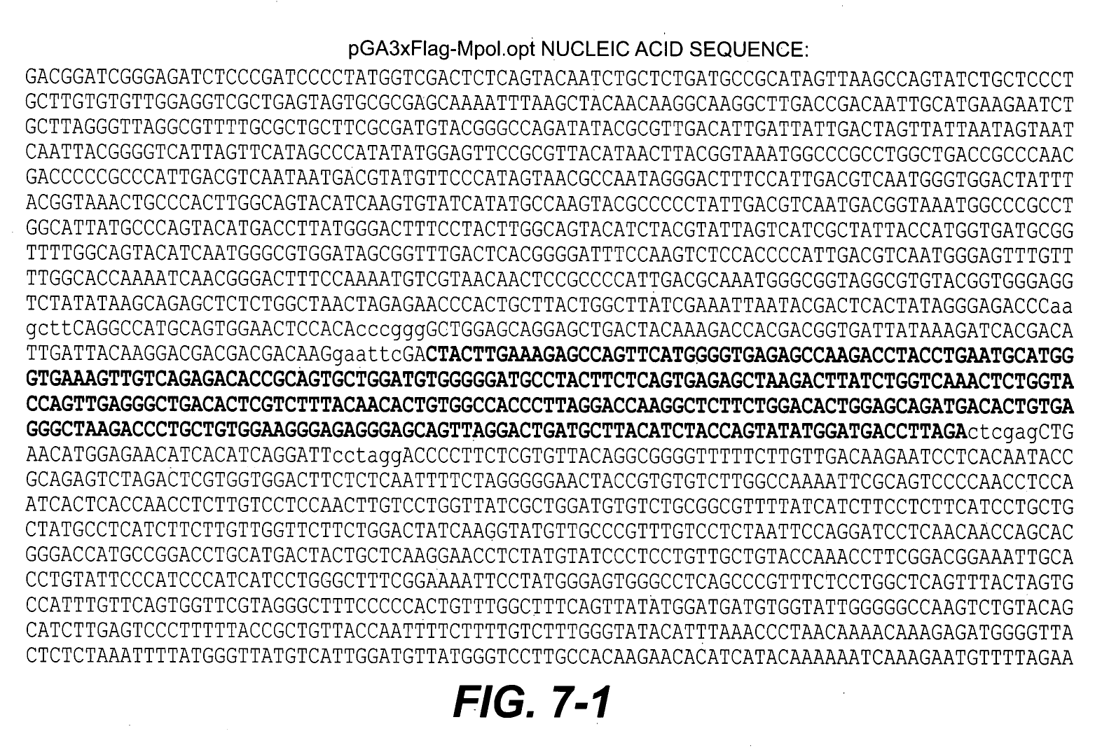

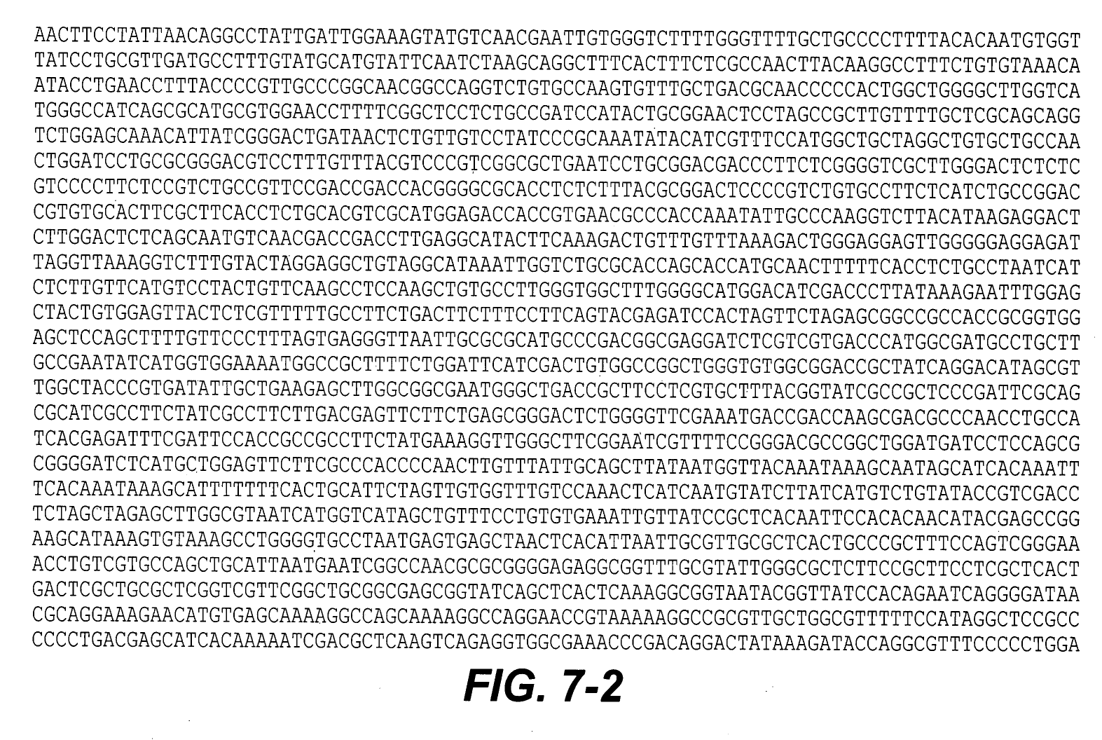

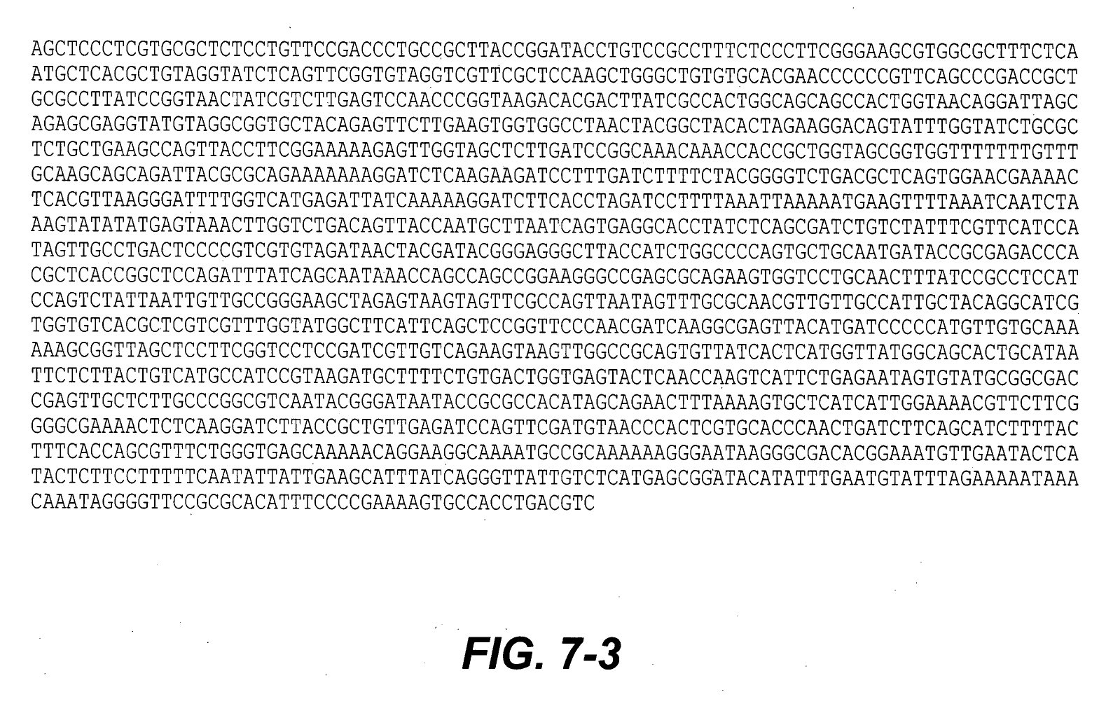

The poIHIV-1.opt polyepitope was cloned through the EcoRI and XhoI restriction sites into the pGA1xFlag-M and pGA3xFlag-M plasmids to obtain plasmid constructions designated pGA1xFlag-MpoI.opt and pGA3xFlag-MpoI.opt, respectively. FIG. 4 is a detailed nucleic acid sequence for the GA1xFlag-MpoI.opt insert in pGA1xFlag-M to produce pGA1xFlag-MpoI.opt. FIG. 5 is a detailed nucleic acid sequence for the GA3xFlag-MpoI.opt insert in pGA3xFlag-M to produce pGA3xFlag-MpoI.opt. FIGS. 6 and 7 show nucleic acid sequences of resulting pGA1xFlag-MpoI.opt and pGA3xFlag-MpoI.opt, respectively. In FIGS. 4 and 5, preS2 and HBsAg ATG codons are highlighted in bold and in FIGS. 6 and 7, the poIHIV-1.opt polyepitope is highlighted in bold. In FIGS. 3, 4, 5 and 6 the EcoRI and XhoI restriction sites are in lower case.

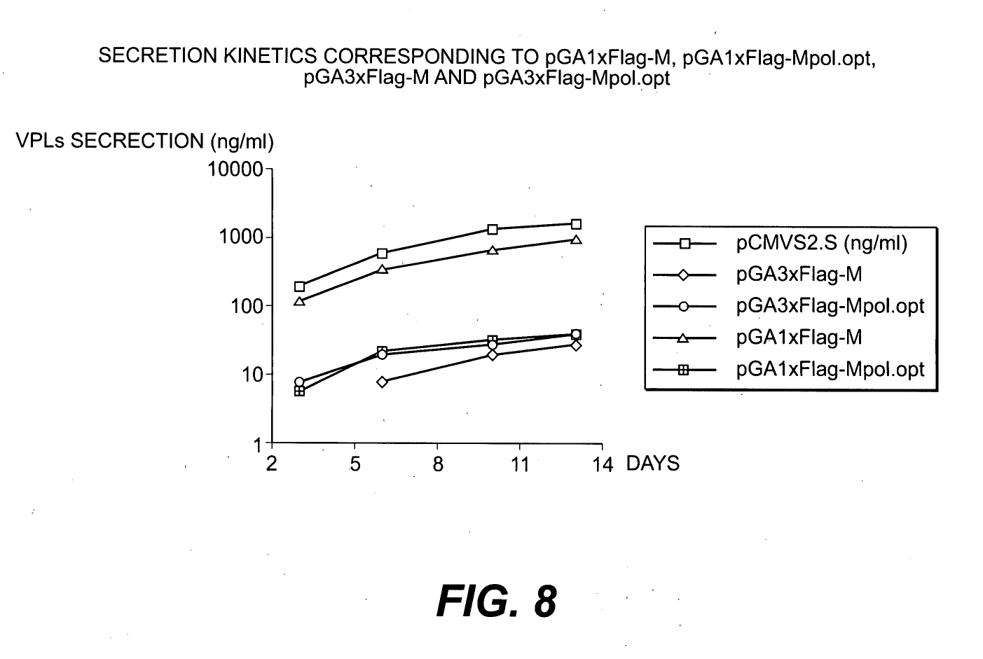

The secretion kinetics corresponding to pGA1xFlag-M, pGA1xFlag-MpoI.opt, pGA3xFlag-M and pGA3xFlag-MpoI.opt are shown in FIG. 8.

Similarly, four additional polyepitopic sequences were designed. These polyepitopic sequences have been designated poI1A2, poI2A2, poI1B7, and poI2B7. The nucleic acid and amino acid sequences, as well as epitope name and epitope sequences, are as follows.

Nucleic and Amino Acid Sequences of poI1A2

GTGCTGGATGTGGGAGATGCCTACTTCTCAGTGAGAGCTGACACCTACCT
GAATGCCTGGGTGAAGGTGGTCAGAGCCAAGACCTACCTGGTGAAGCTGT
GGTACCAGCTGAGGACAGATGCCTCCCTGGTGAAGCATCACATGTATGTG
AGAGACACAGCCTACATCTACCAGTACATGGATGACCTGAGA
VLDVGDAYFSVRADTYLNAWVKVVRAKTYLVKLWYQLRTDASLVKHHMYV
RDTAYIYQYMDDLR

| | | |
|---|---|---|
| Name | aa seq | nuc seq |

| | | |
|---|---|---|
| V11V | VLDVGDAYFSV | TGCTGGATGTGGGAGATGCCTACTTCTCAGTG |
| Y/T9V | YLNAWVKVV | TACCTGAATGCCTGGGTGAAGGTGGTC |
| Y/P9L | YLVKLWYQL | TACCTGGTGAAGCTGTGGTACCAGCTG |
| Vif23 | SLVKHHMYV | TCCCTGGTGAAGCATCACATGTATGTG |
| V/V9L | YIYQYMDDL | TACATCTACCAGTACATGGATGACCTG |

Nucleic and Amino Acid Sequences of poI2A2

CTGCTTGACACAGGAGCTGATGACACAGTGAGGACAGATGCCAGCCTGTA
TAACACAGTGGCCACCCTGAGAGCTGACACCTACCTGAAGGAGCCTGTGC
ATGGAGTGAGAGCTAAGACCCTCCTGTGGAAGGGAGAGGGAGCAGTGAGA
ACCAAGGCAGTGCTGGCTGAGGCCATGTCCCAGGTGAGA
LLDTGADDTVRTDASLYNTVATLRADTYLKEPVHGVRAKTLLWKGEGAVR
TKAVLAEAMSQVR

| | | |
|---|---|---|
| Name | aa seq | nuc seq |

| | | |
|---|---|---|
| L10V | LLDTGADDTV | CTGCTTGACACAGGAGCTGATGACACAGTG |
| S9L | SLYNTVATL | AGCCTGTATAACACAGTGGCCACCCTG |
| Y/I9V | YLKEPVHGV | TACCTGAAGGAGCCTGTGCATGGAGTG |
| L9V | LLWKGEGAV | CTCCTGTGGAAGGGAGAGGGAGCAGTG |
| Gag 362 | VLAEAMSQV | GTGCTGGCTGAGGCCATGTCCCAGGTG |

Nucleic and Amino Acid Sequences of poI1B7

TCCCCTAGGACCCTGAATGCCTGGGTGAGAGCTAAGACCAGACCTAACAA
TAACACAAGGAAGTCCATCAGAGACACAGCCTTCCCTGTGAGACCACAGG
TGCCTCTGAGGAGAACCAAGGCCCACCCTGTGCATGCTGGCCCTATTGCC
AGAGCTGATACAGCACCCACTAAGGCCAAAAGGAGAGTGGTCAGG
SPRTLNAWVRAKTRPNNNTRKSIRDTAFPVRPQVPLRRTKAHPVHAGPIA
RADTAPTKAKRRVVR

| | | |
|---|---|---|
| Name | aa seq | nuc seq |

| | | |
|---|---|---|
| S9WV | SPRTLNAWV | TCCCCTAGGACCCTGAATGCCTGGGTG |
| R10SI | RPNNNTRKSI | AGACCTAACAATAACACAAGGAAGTCCATC |
| F10LR | FPVRPQVPLR | TTCCCTGTGAGACCACAGGTGCCTCTGAGG |
| Gag 237 | HPVHAGPIA | CACCCTGTGCATGCTGGCCCTATTGCC |
| A10VC | APTKAKRRVV | GCACCCACTAAGGCCAAAAGGAGAGTGGTC |

Nucleic and Amino Acid Sequences of poI2B7

AAGCCTGTGGTCTCCACACAGCTGCTTCTCAGGGCCAAGACCTTCCCTGT
GAGACCCCAAGTGCCACTGAGAAGGGCTGATACACAGCCCAGGAGTGACA
CCCATGTGTTCAGAACCAAGGCCATTCCTAGGAGAATTAGGCAGGGCCTG
AGAGATACAGCTACACCTCAGGACCTGAACACCATGCTGAGA
KPVVSTQLLLRAKTFPVRPQVPLRRADTQPRSDTHVFRTKAIPRRIRQGL
RDTATPQDLNTMLR

| | | |
|---|---|---|
| Name | aa seq | nuc seq |

| | | |
|---|---|---|
| K10LL | KPVVSTQLLL | AAGCCTGTGGTCTCCACACAGCTGCTTCTC |
| F10LR | FPVRPQVPLR | TTCCCTGTGAGACCCCAAGTGCCACTGAGA |
| Q9VF | QPRSDTHVF | CAGCCCAGGAGTGACACCCATGTGTTC |
| I9GL | IPRRIRQGL | ATTCCTAGGAGAATTAGGCAGGGCCTG |
| T9ML | TPQDLNTML | ACACCTCAGGACCTGAACACCATGCTG |

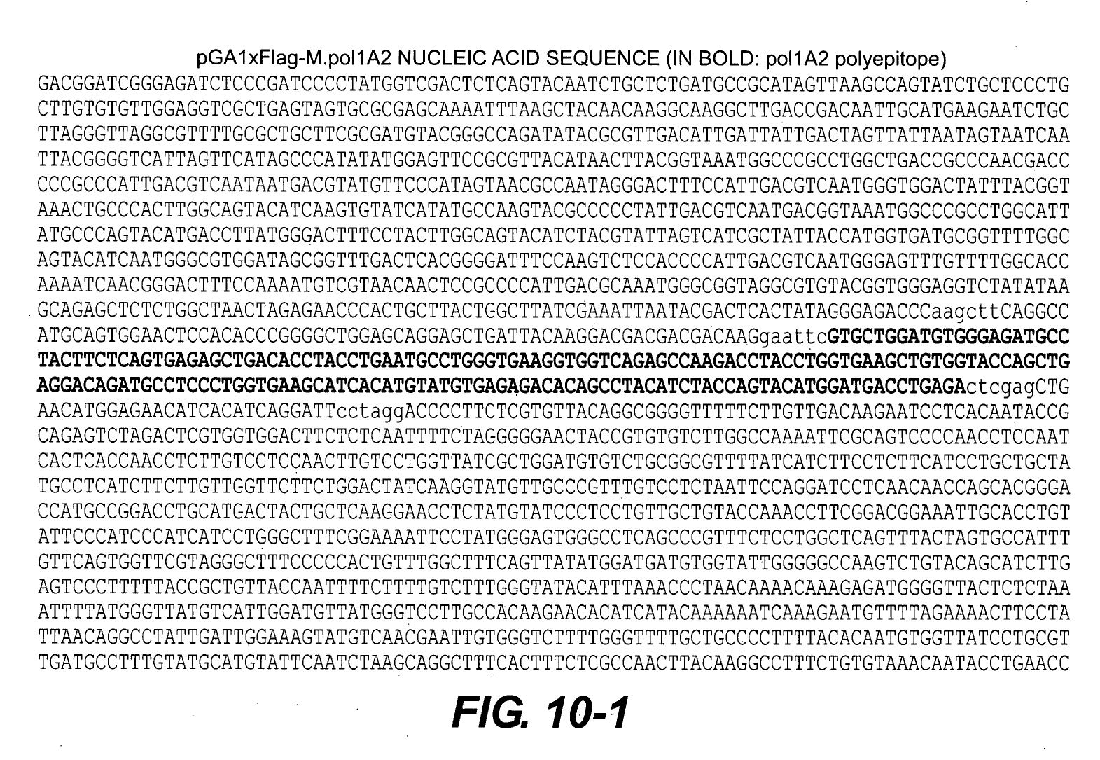

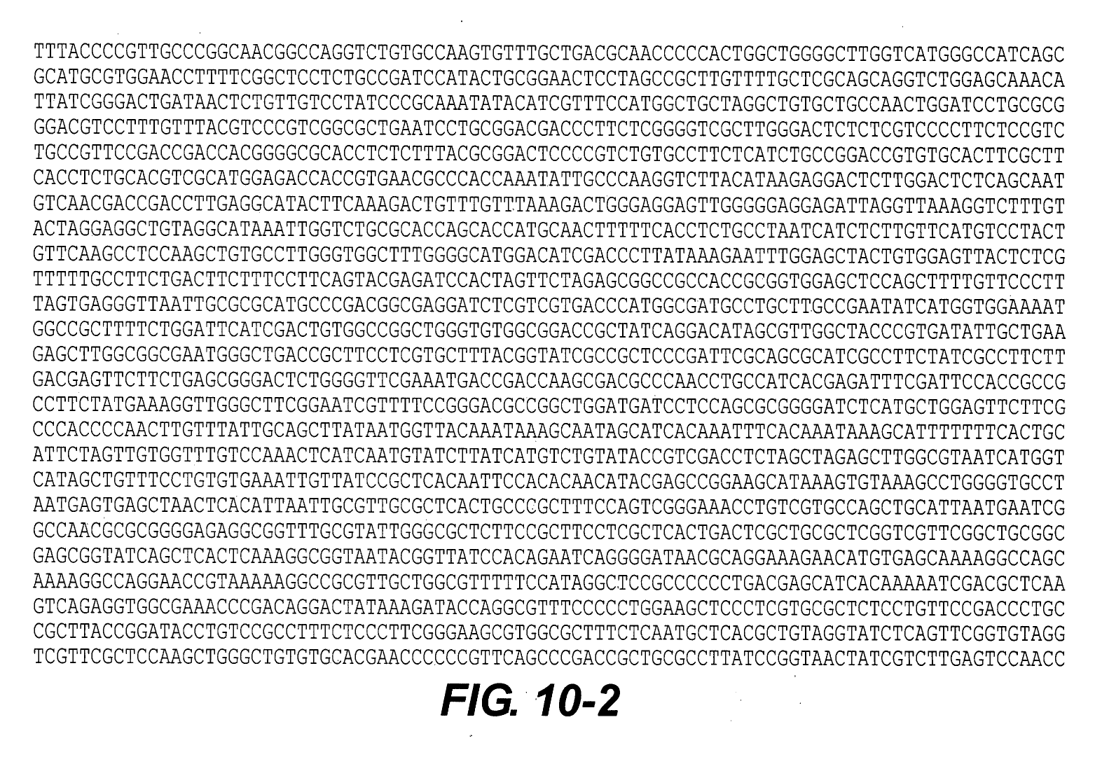

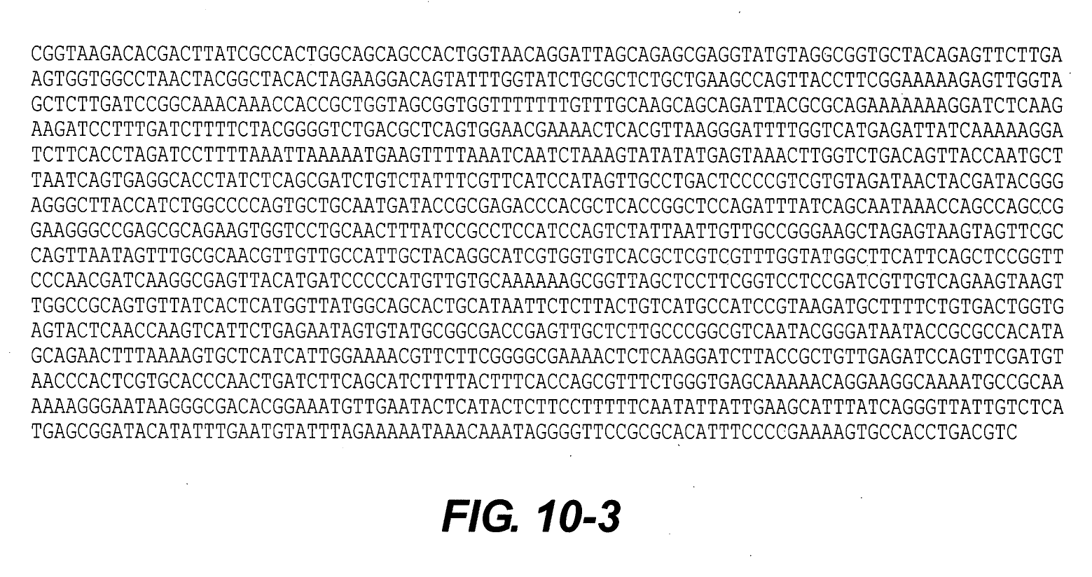

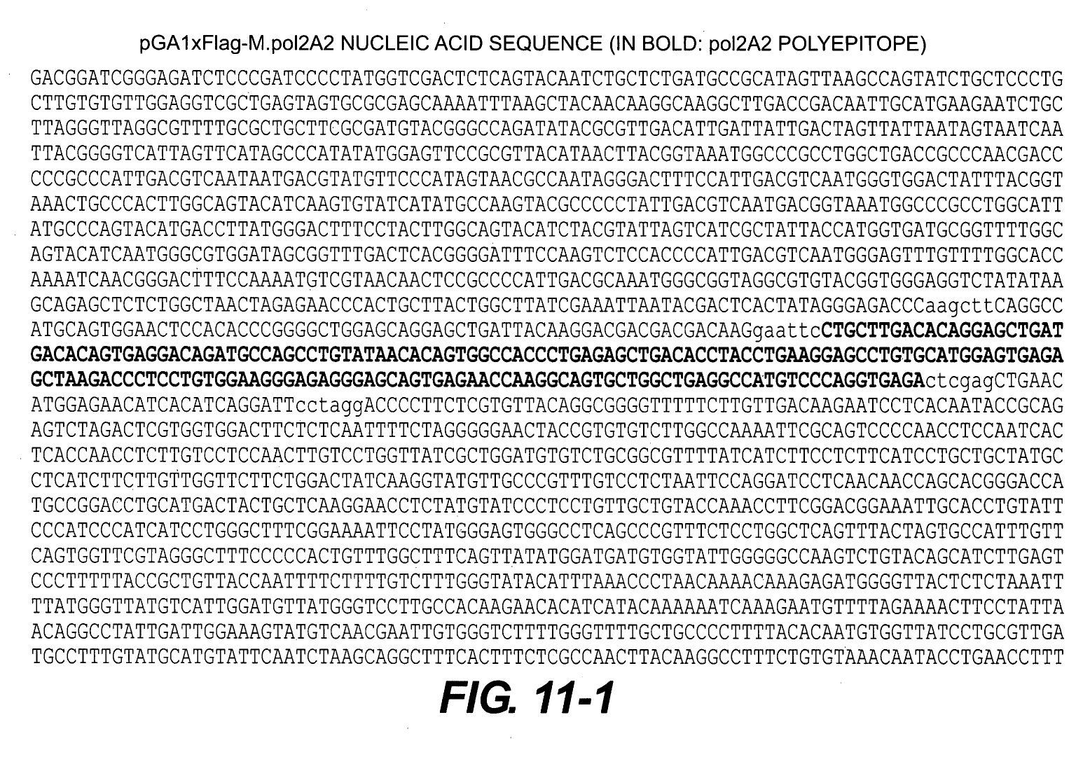

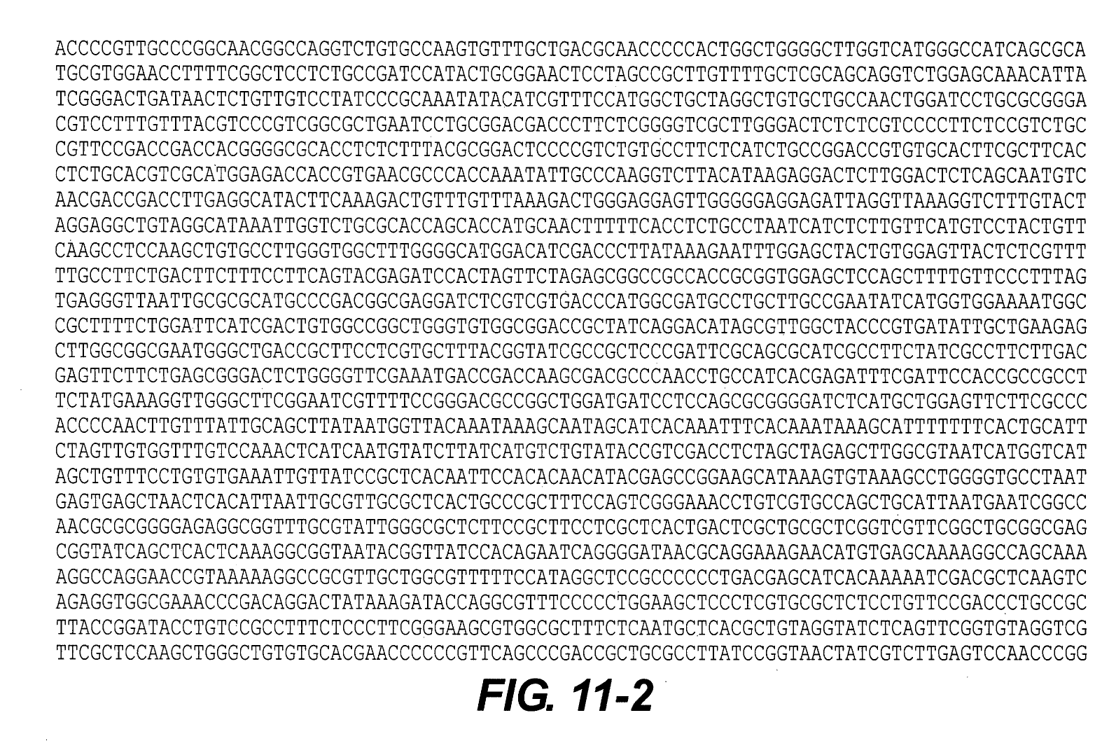

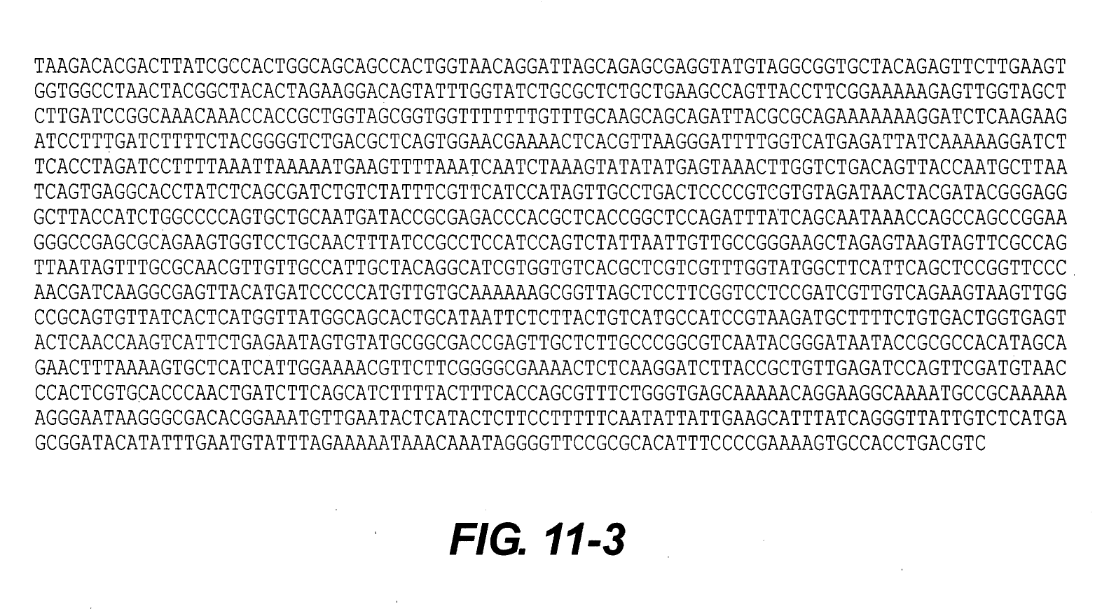

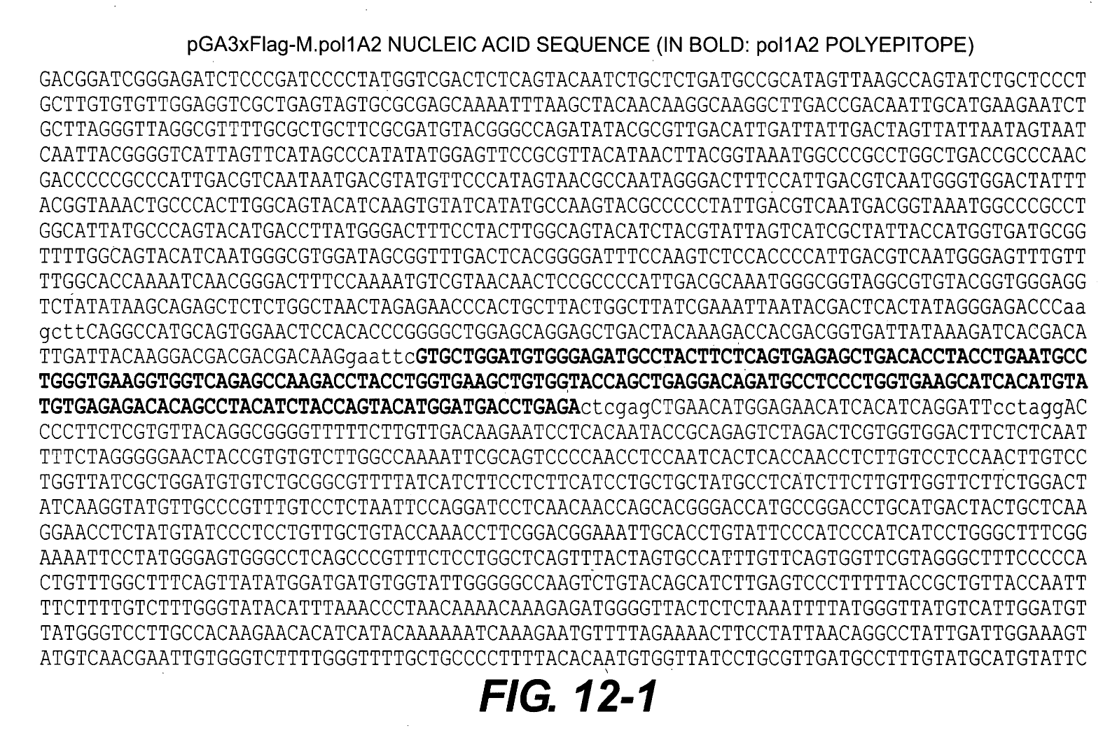

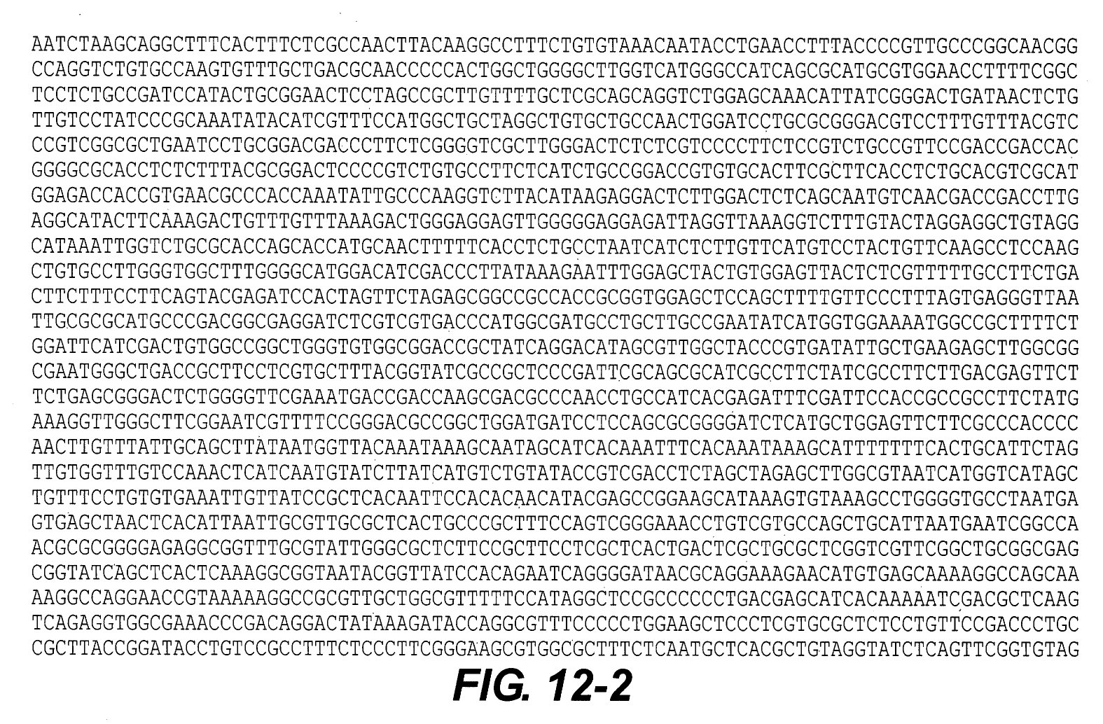

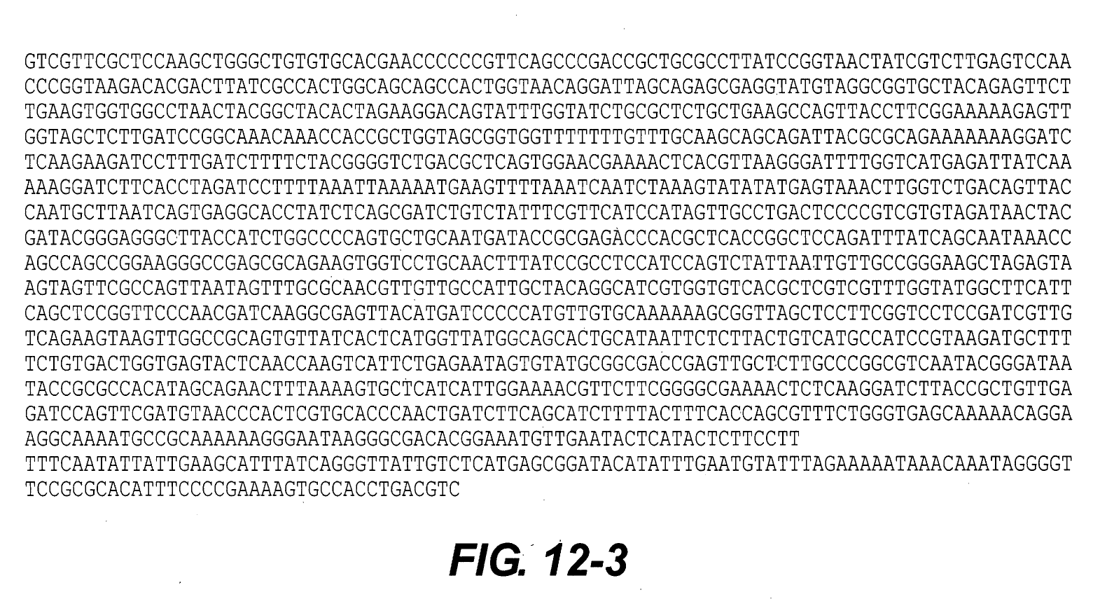

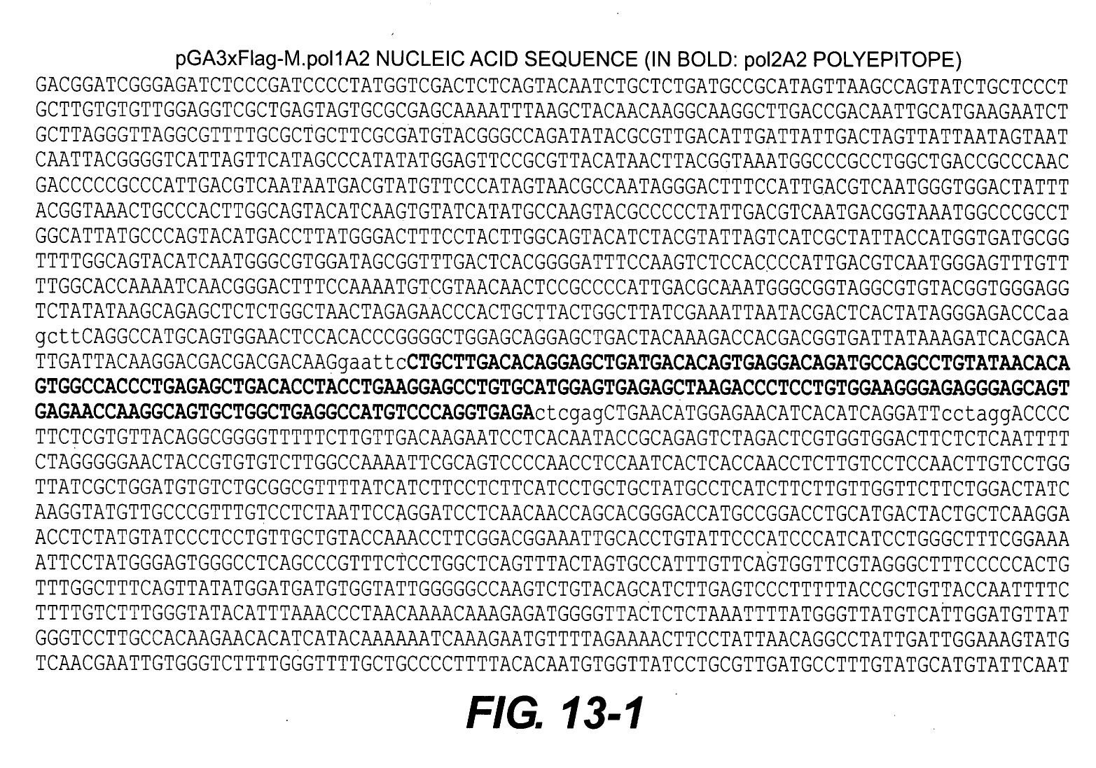

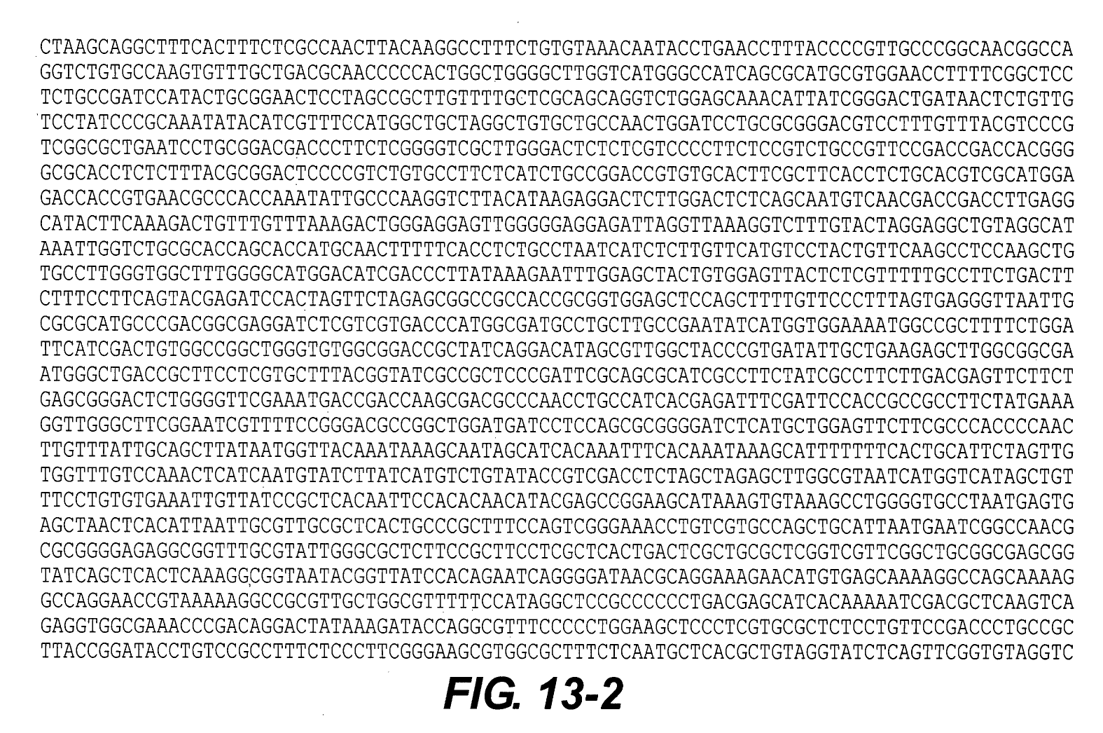

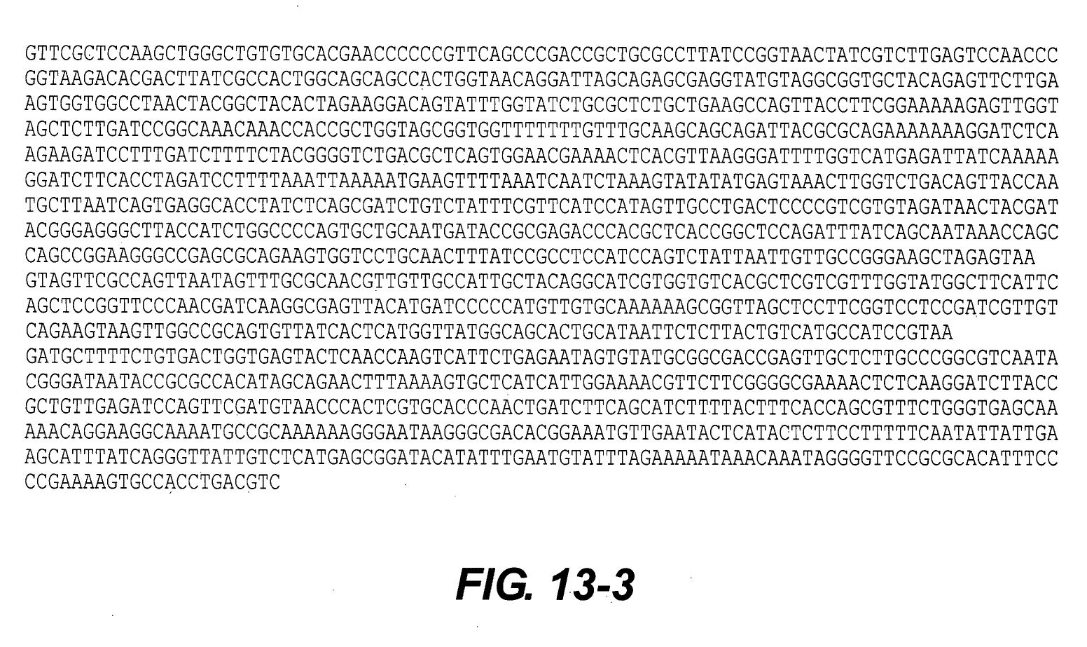

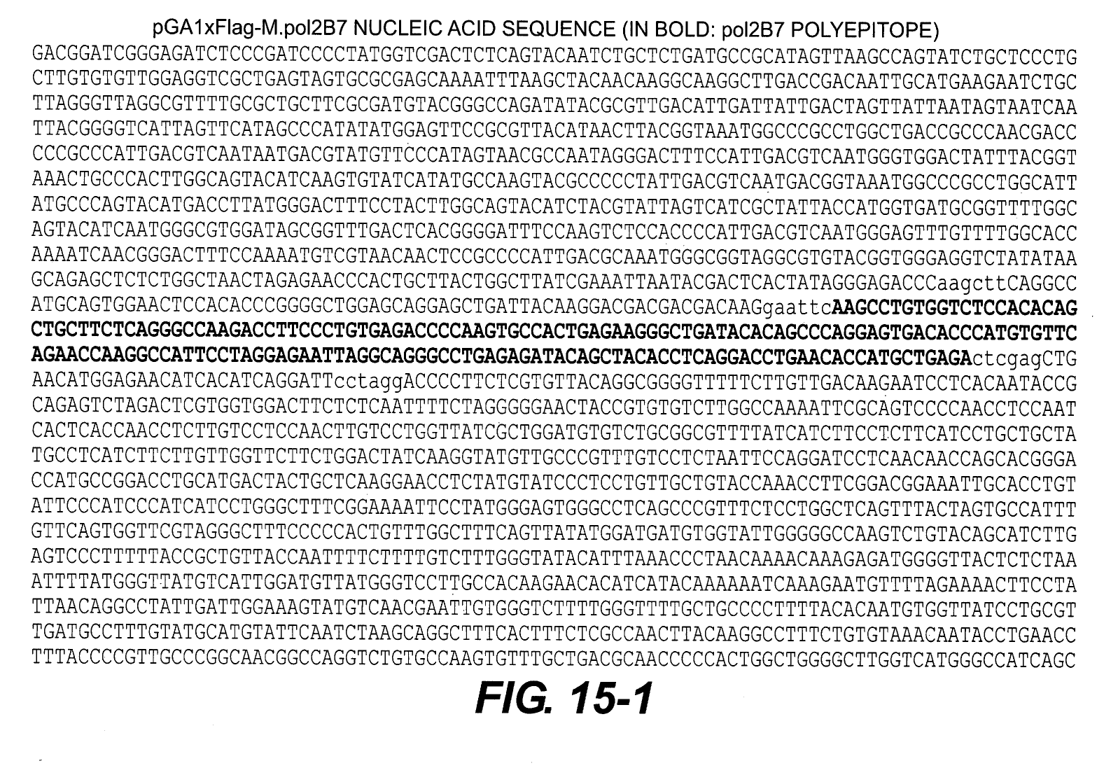

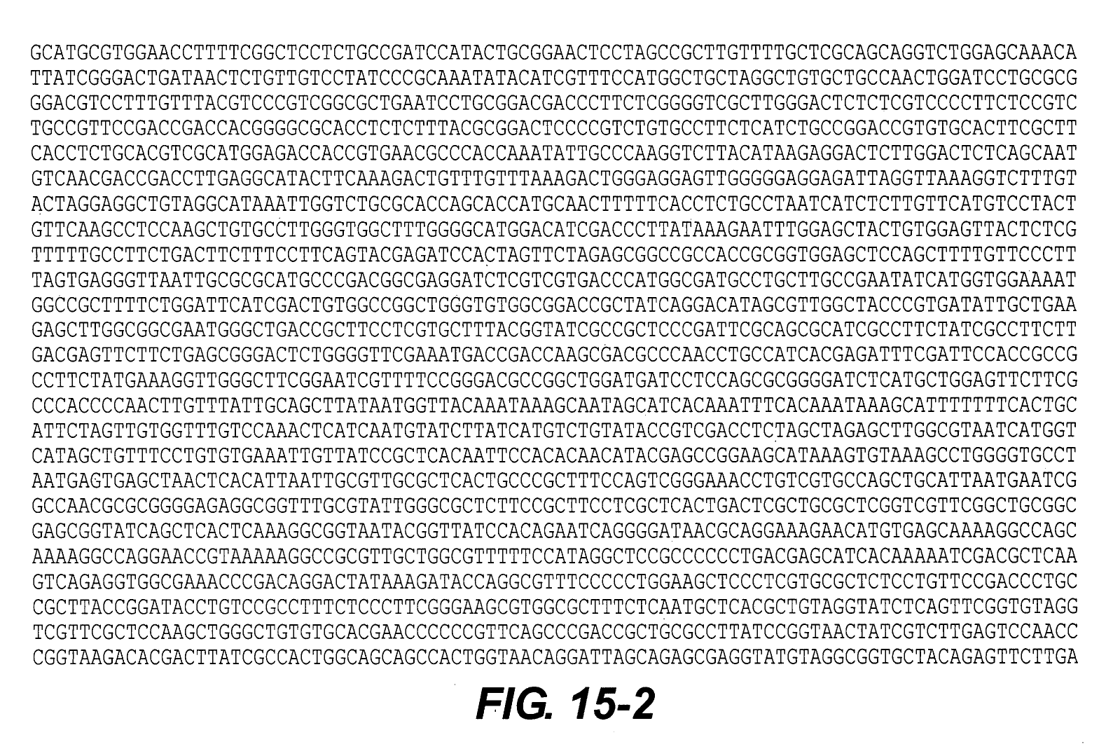

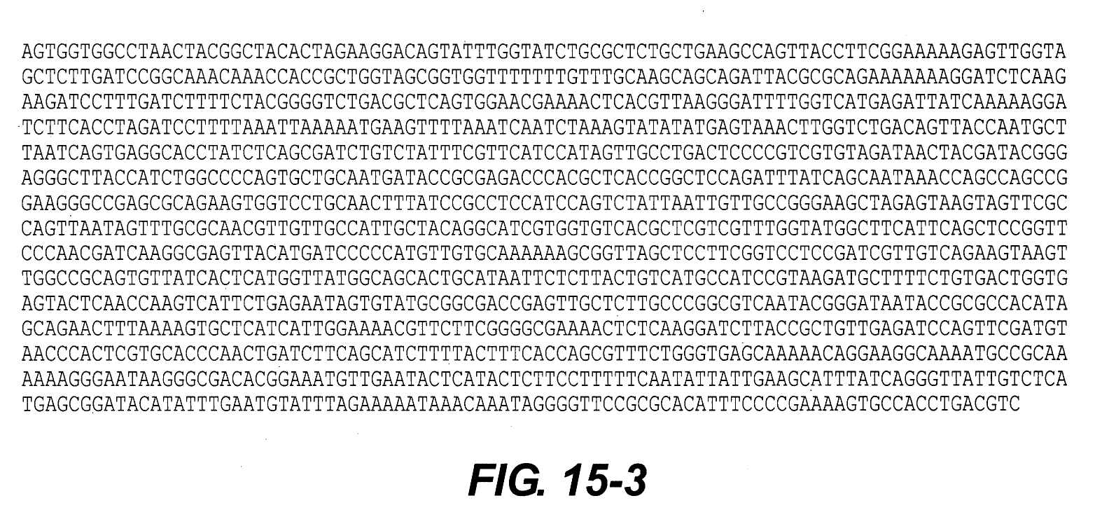

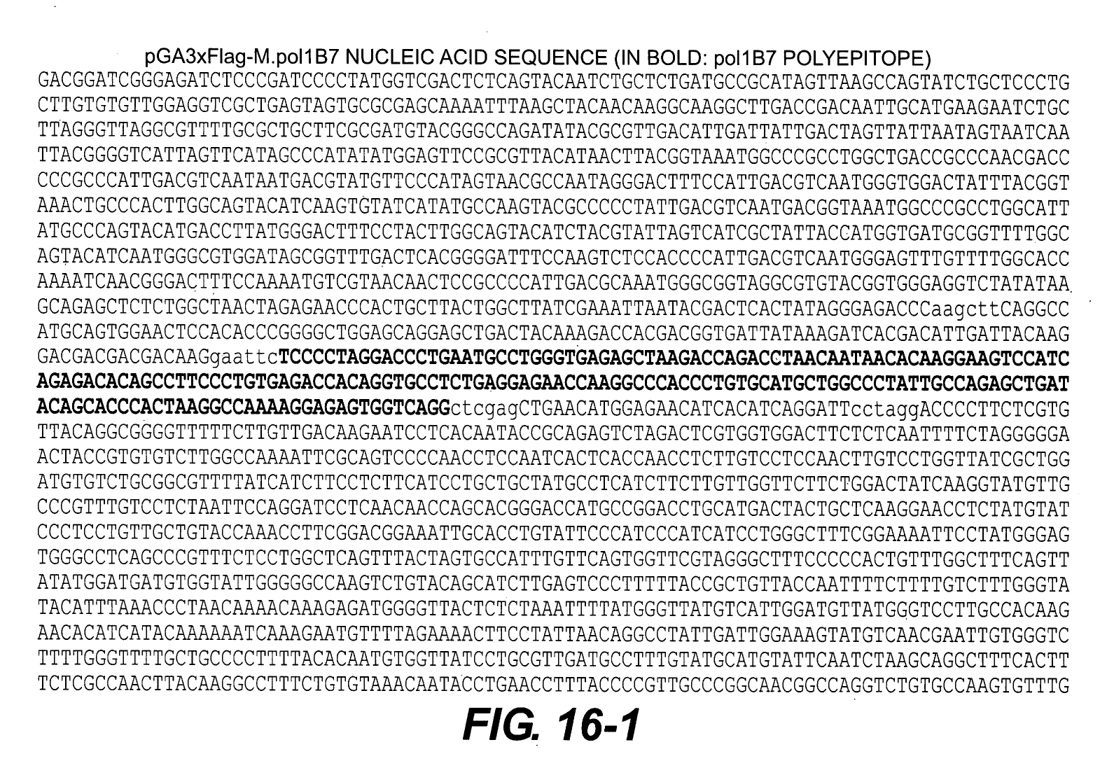

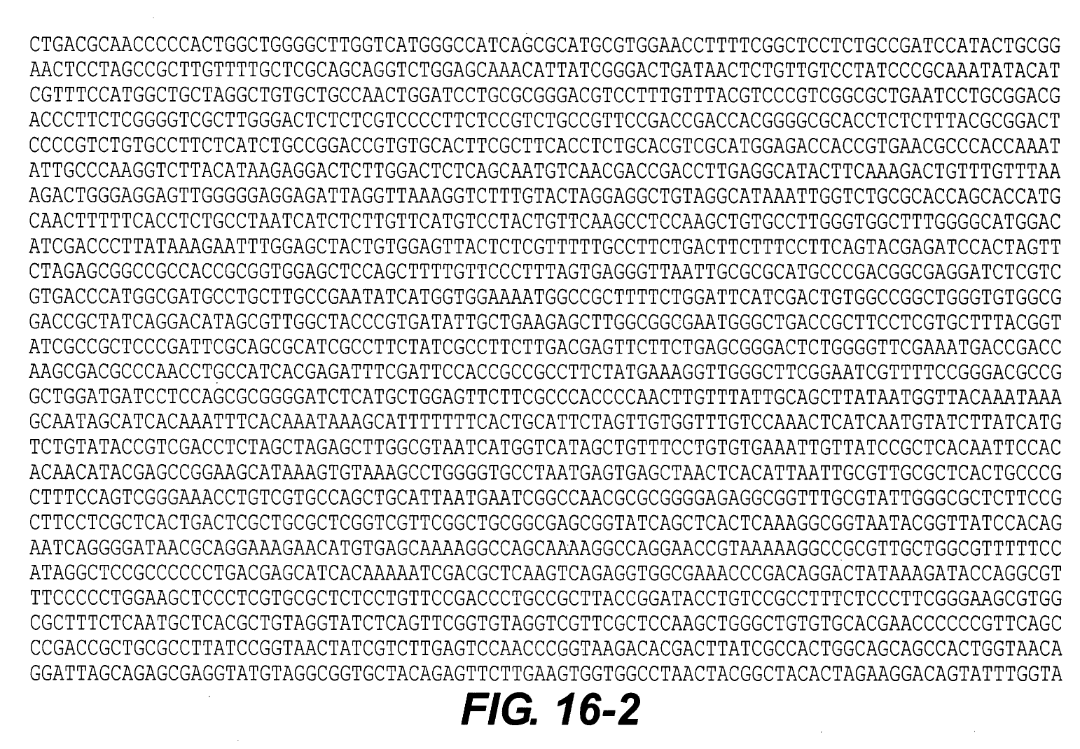

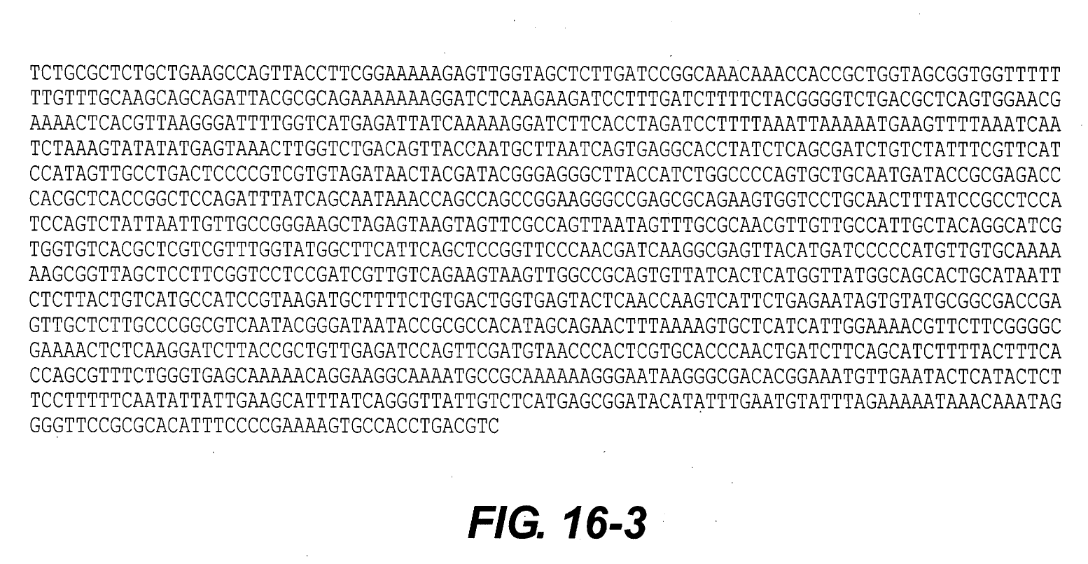

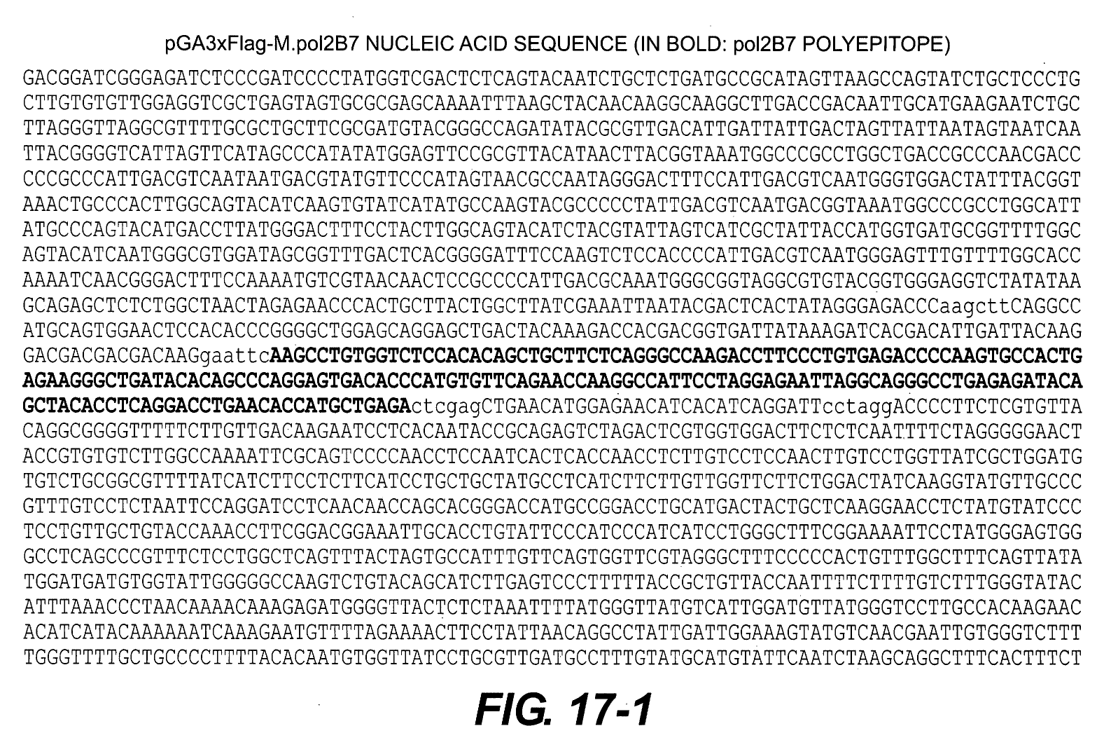

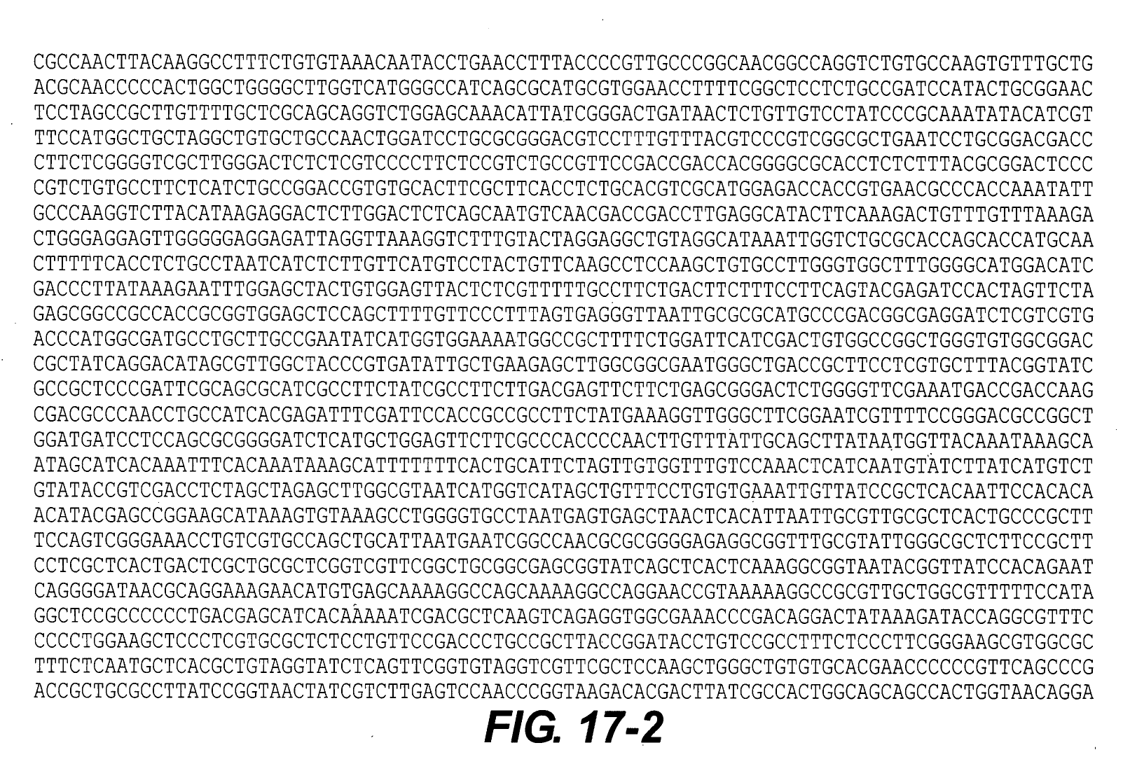

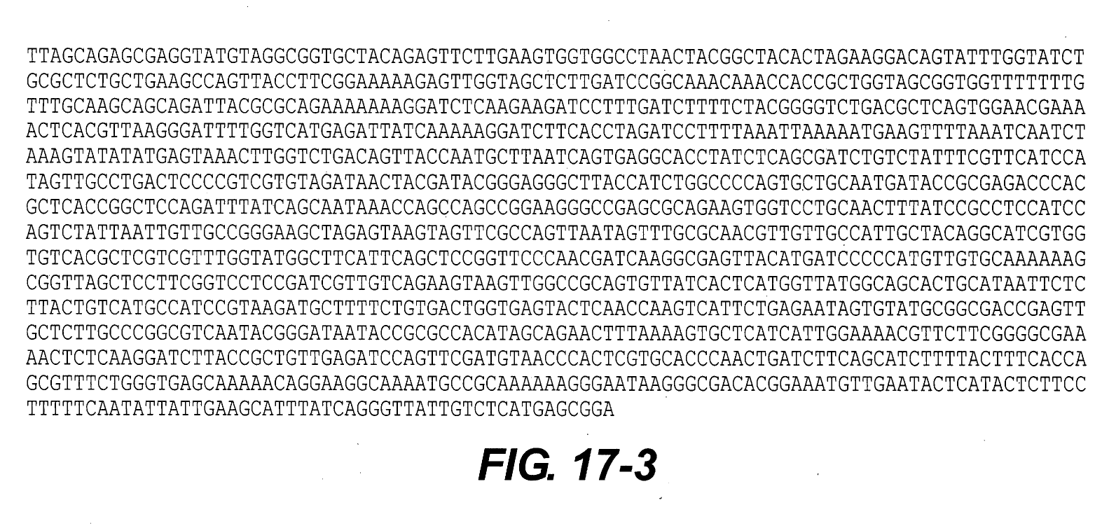

Each of the polyepitopes poI1A2, poI2A2, poI1B7, and poI2B7 was similarly inserted into pGA1xFlag-M and pGA3xFlag-M plasmids. A detailed nucleic acid sequence for each of the resulting constructs is shown in FIGS. 10 to 17. The polyepitopic sequence inserted in the plasmid is shown in bold in each Figure.

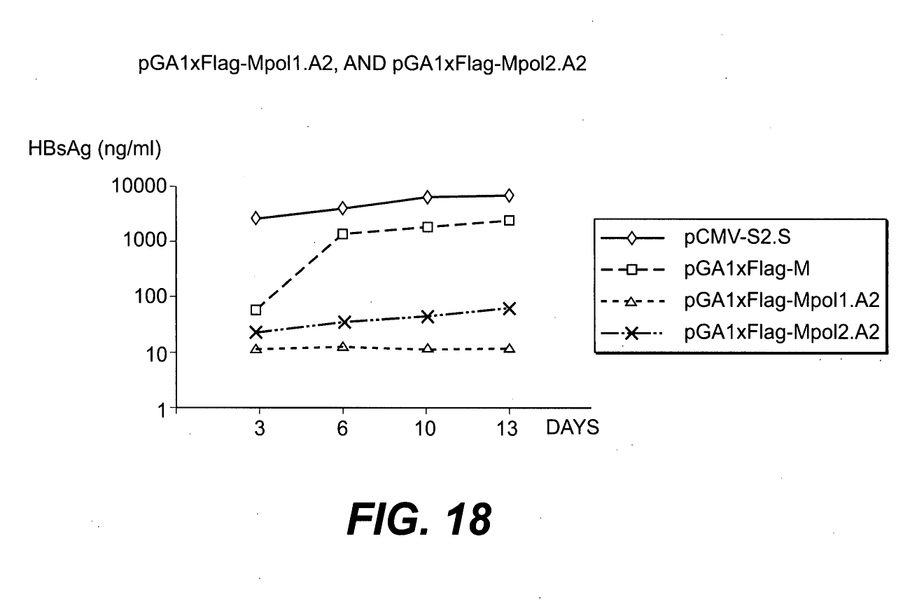

The recombinant HBsAg VLPs secretion kinetics corresponding to pGA1xFlag-M.poI1A2, pGA1xFlag-M.poI2A2, pGA3xFlag-M.poI1A2, pGA3xFlag-M.poI2A2, pGA1xFlag-M.poI1B7, pGA1xFlag-M.poI2B7, pGA3xFlag-M.poI1B7, and pGA3xFlag-M.poI2B7 transfections are shown in FIGS. 18 to 21. All constructions give rise to VLPs secretion from transfected cells. The lowest values are obtained by poll B7 and poI2B7 bearing constructions. This is due to the fact that HLA-B7 restricted epitopes are more hydrophobic peptides, when compared to HLA-A2 restricted ones.

All in vitro analyses employed the pCMV-S2.S positive control plasmid, which expresses the wild type preS2-HBsAg fusion protein (23).

This invention will now be further described in the following Examples.

### EXAMPLE 1

### Expression Vectors and Constructions

The plasmid referred to as pCMV-basic (FIG. 1B) is a derivative of the pCMV-B10 construction. It was made with a small polylinker (NheI, EcoRV, SmaI) replacing the pCMV-B10 polylinker between the EcoRI and XhoI restriction sites.

The pGA1xFlag-M and pGA3xFlag-M plasmids are based on the expression vector pCMV-basic plasmid backbone (FIG. 1B) and obtained by substitution of the nucleic acids sequence in between the HindIII and the AvrII restriction sites by the insertion of the GA1xFlag-M and GA3xFlag-M motifs. In the GA1xFlag-M and GA3xFlag-M motifs, starting from the HindIII restriction site in 5′, were introduced the QA peptide corresponding to the C-terminal sequence of the HBV preS1 region, the MQWNSTP peptide corresponding to the N-terminal portion of the HBV preS2 protein. This latter introduced into the construction the glycosylation site (N₄ ), which is highly conserved among primate HBV isolates. The preS2 peptide is followed by the GA motif (Gly-Ala amino acids repeated three time), here located to prevent steric impairment for the binding of anti-Flag antibodies to the tag (1xFlag-M or 3xFlag-M) by sugar molecules covalently linked to preS2 N₄*. The GA motif is followed by the 1xFlag-M or 3xFlag-M tags (modified from SIGMA-ALDRICH), giving the pGA1xFlag-M and pGA3xFlag-M, respectively (FIGS. 2 and 3). Nucleic sequences of the original SIGMA-ALDRICH 1xFlag and 3xFlag have been modified preserving amino acids sequences but eliminating methionine residues in secondary and tertiary phases. In between the EcoRI and XhoI restriction sites, a new polylinker has been inserted, where the PstI, NheI, BgIII restriction sites follow one the others. In the new constructions, the preS2 C-terminal peptide preceding the HBsAg methionine start codon (M₁) has been reduced to two amino acids (Leu-Asn), preserving the nucleic acid context, hence the strength, of the HBsAg ATG codon. Finally several N-terminal amino acid residues of HBsAg (ENITSG) were introduced.

The two constructions have been obtained by “atypical PCR” using long primers (60-80 nucleic acids) detailed in the Table I (nucleic acids in HindIII and AvrII restriction sites are highlighted in small case).

TABLE 1

Oligonucleotides used for GA1XFlag-M and
GA3XFlag-M motifs construction

| | |
|---|---|
| Oligonucleotide | Sequence |

| | |
|---|---|
| 1Xflag1 | 5′-CAGGCCATGCAGTGGAACTCCACACCCGGGG |
| | CTGGAGCAGGAGCTGATTACAAGGACG-3′ |
| 1Xflag2 | 5′-TGGAGCAGGAGCTGATTACMGGACGACGACG |
| | ACAAGgaattcCTGCAGGCTAGCAGATCTct |
| | cg-3′ |
| Xflag3 | 5′-tcCGTGCAGGCTAGCAGATCTctcgagCTGA |
| | ACATGGAGAACATCACATCAGGATTcctag-3′ |
| 3Xflag-M.1 | 5′-CAGGCCATGCAGTGGACTCCACACCCGGGGC |
| | TGGAGCAGGAGCTGACTACAAG-3′ |
| 3Xflag-M.2 | 5′-TGGAGCAGGAGCTGACTACAAGACCACGACG |
| | GTGATTATAAAGATCACGACATfGA1TACAG-3′ |
| 3Xflag-M.3 | 5′-TGATTATAAGATCACGACATTGATTACAGGA |
| | CGACGACGACAGgaattcCTGCAGGCTAGCAGAT |
| | CTctcg-3′ |
| 3Xflag-M.4 | 5′-TGCAGGCTAGCAGATCTctcgagCTGACATG |
| | GAGACATCACATCAGGATTcctag-3′ |
| 5′ flag | 5′-AGACCCaagcttCAGGCCATGCAGTGGAACT |
| | CCACA-3′ |
| 3′ flag | 5′-AGGGGTcctaggATCCTGATGTGATG1TCTC |
| | CATG-3′ |

Two separate reactions (A and B), for GA1xFlag-M and GA3xFlag-M motifs respectively, were performed using 50 pmols of 1xFlag1, −2 and −3, in reaction A, and 3xFlag-M .1, .2, .3, and .4 in B. In both reactions A and B, 10 pmols 3′ flag were added. Six cycles of PCR were then performed.

Then, 25 cycles of classical PCR were performed, adding 100 pmols of the 5′ flag and the 3′ flag primers.

To obtain the pGA1xFlag-M and pGA3xFlag-M plasmids, the reaction A and B PCR fragments were then digested by HindIII and AvrII to be cloned into the corresponding cloning sites of the pCMV-basic plasmid.

To obtain the pGA1xFlag-MpoI.opt and pGA3xFlag-MpoI.opt plasmids (FIGS. 6 and 7), the poIHIV-1.opt polyepitope was cloned between the EcoRI and XhoI restriction sites of the pGA1xFlag-M and pGA3xFlag-M plasmids, respectively. Codon usage was optimized according to the Homo sapiens table (http://www.kazusa.or.ip/codon). Hydrophathy profiles were obtained by DNA Strider™ 1.2 (Kyte-Doolittle option).

The poIHIV-1.opt polyepitope was assembled by multiple rounds of “atypical PCR”. Briefly, a series of six 70-80-mer oligonucleotides were synthesized corresponding to the plus strand and overlapped one another by ˜20 bases at both 5′ and 3′ ends (the oligonucleotides used in this invention are shown in Table 2).

TABLE 2

Oligonucleofides used for polHIV-1.opt
polyepitope construction

| | |
|---|---|
| Oligonucleotide | Sequence |

| | |
|---|---|
| 1-HIVPOLY | 5′-GAATTCCTACTTGAAAGAGCCAGTICATGGG |
| | GTGAGAGCCPAGACCTACCTGAATGCATGGGTGA |
| | AGTTG-3′ |
| 2-HIVPOLY | 5′-CTGAATGCATGGGTGAAAGTTGTCAGAGACA |
| | CCGCAGTGCTGGATGTGGGGGATGCCTACTTCTC |
| | AGTGAGAG-3′ |
| 3-HIVPOLY | 5′-ATGCCTACTTCTCAGAGAGAGCTAAGACTTA |
| | TCTGGTCAAACTCTGGTACCAGTTGAGGGCTGAC |
| | ACTCG-3′ |
| 4-HIVPOLY | 5′-CAGTTGAGGGCTGACACTCGTCTTTACAACA |
| | CTGTGGCCACCCTTAGGACCAAGGCTCTTCTGGA |
| | CACTGGAGCAGATG-3′ |
| 5-HIVPOLY | 5′-CTTCTGGACACTGGAGCAGATGACACTGTGA |
| | GGGCTAAGACCCTGCTGTGGAAGGGAGAGGGAGC |
| | AGTTAGGACTG-3′ |
| 6-HIVPOLY | 5′-AAGGGAGAGGGAGCAGTTAGGACTGATGCTT |
| | ACATCTACCAGTATATGGATGACCTTAGACTCGA |
| | G-3′ |
| 5′ conPmodifpc | 5′-CATGAACTGGCTCTTTCAAGTAGGAATTCCA |
| | CTG-3′ |
| 5′ modifPoly | 5′-GCAGTGGATTCCTACTTGAAAGAGCCAGTTC |
| | ATG-3′ |
| 3′ modifPoly | 5′-CTATATGCTCGAGTCAAGGTCATCCATATAC |
| | TG-3′ |

Two separate reactions (A and B) were performed using 50 pmols of HIVPOLY-1, -2 and -3, in reaction A, and HIVPOLY-4, -5 and -6 in B, respectively. Then, 25 pmols of 5′conpmodifpc and 3′modifpoly were added in reactions A and B, respectively. Fifteen cycles of PCR were then performed.

PCR products from reactions A and B were assembled as follows: 0.5 μl of each reaction were put in 20 μl of H₂O at 95° C. for 30 seconds and then to room temperature (r.t.). Five units of Klenow fragment and 1 μl of dNTPs (40 mM) were added and reaction performed for 15 minutes at 37° C.

Then, 25 cycles of classical PCR were performed, adding 100 pmols of the 5′modifpoly and the 3′modifpoly primers.

### EXAMPLE 2

### In Vitro Evaluation of VLPs Secretion by the Plasmids Bearing the poIHIV-1.opt Polyepitope

The pGA1 xFlag-M, pGA3xFlag-M, pGA1 xFlag-MpoI.opt and pGA3xFlag-MpoI.opt plasmids were transiently transfected into SW480 cells, along with pCMV-S2.S as positive control for HBsAg VLPs formation and secretion (FIG. 8). The pCMV-S2.S plasmid expresses the wild type pres2-HBsAg fusion protein (23).

More particularly, the SW480 human cell line was maintained in Dulbecco medium supplemented with 5% foetal calf serum (FCS) and 1% streptomycin and penicillin, according to recommendations of the manufacturer. The pCMV-S2.S plasmid was kindly provided by Dr. Marie-Louise Michel (23).

Cells were transiently transfected by FuGENE6™ transfection reagent (Roche). Out of 2 ml, 500 μl of supernatant were collected and renewed at each time point. HBsAg concentration in supernatants was estimated by the Monolisa® Ag HBsAg Plus Kit (BIORAD). To detect fusion protein on HBsAg VLPs, an anti-Flag-M ELISA was performed using the M2 monoclonal antibody (SIGMA-ALDRICH), which recognizes a linear epitope. Briefly, 96 well plates were coated with M2 mAb, and 2.5 ng/ml of HBsAg positive samples tested per well.

The anti-HBsAg ELISA test used allows detection and quantification of HBsAg antigenic units only if the protein is assembled into VLPs. The pGA1xFlag-M plasmid resulted in VLPs secretion, which was only ˜2-3 fold down from the pCMV-S2.S (FIG. 8). The pGA1xFlag-MpoI.opt plasmid resulted in VLPs secretion ˜20-35 fold down from the pGA1xFlag-M. These data clearly show a gradual impact of HBsAg fusion protein complexity on the inhibition of recombinant VLPs assembly. This impact is more drastic in the case of the pGA3xFlag-M, which resulted in VLPs secretion ˜60-70 fold down from the pCMV-S2.S. Nevertheless, over a 14 days period, recombinant HBsAg VLPs could be detected at comparable levels in pGA1xFlag-MpoI.opt and pGA3xFlag-MpoI.opt samples. This suggests that once the poIHIV-1.opt polyepitope is inserted into the pGA1xFlag-M or the pGA3xFlag-M plasmid, the inhibition by the GA1xFlag-M and GA3xFlag-M motifs on VLPs assembly is eliminated and replaced by the impact of polyepitope next to the N-terminal ATG of the HBsAg, here of the polHIV-1.opt polyepitope.

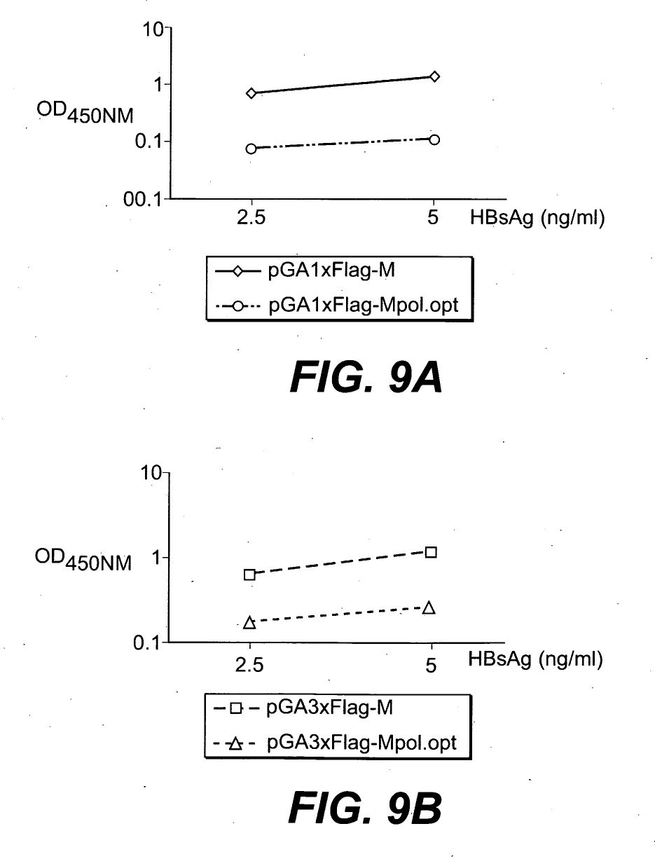

To verify that the pGA1xFlag-M, pGA3xFlag-M, pGA1xFlag-MpoI.opt and pGA3xFlag-MpoI.opt plasmids could give rise to recombinant VLPs bearing fusion proteins, an anti-Flag-M ELISA assay was performed. (FIG. 9.) The Flag-M is a linear epitope present in N-terminal to fusion proteins, hence N-terminal to the poIHIV-1.opt polyepitope and the HBsAg genes, in the order. The Flag-M ELISA was performed on the equivalent of 2.5 and 5 ng HBsAg/ml of supernatants determined by the Monolisa® Ag HBsAg Plus Kit (BIORAD). The Flag-M ELISA was made by coating over night 96 well plates with 200 μl of 4 μg/ml of M2 monoclonal anti-Flag antibody (SIGMA-ALDRICH). Supernatants are incubated for 2 hours at 37° C. and then revealed by the R6-R7-R8-R9 reagents from the Monolisa® Ag HBsAg Plus Kit (BIORAD). By this procedure, recombinant VLPs bearing fusion proteins are trapped in ELISA plate wells by the anti-Flag antibody and then identified by polyclonal anti-HBsAg antibodies recognizing conformational epitopes in the VLP structure. As negative samples, N-terminal Flag-BAP™ and 3xFlag-BAP™ control proteins (SIGMA-ALDRICH) and supernatants from pCMV.S2.S transfected cultures are used. Wells were revealed by alkaline phosphatase reaction and read at 450 nm and 620 nm. Limit of detection corresponds to 0.05 OD at 450 nm.

Results showed that the all recombinant VLPs quantified by anti-HBsAg ELISA bear on their surfaces fusion proteins, which are highlighted by the presence of the Flag-M tag (FIG. 9 A and B). This semi-quantitative test allows comparison of GA1xFlag-M (FIG. 9A) or GA3xFlag-M (FIG. 9B) samples as far as their fusion protein content with respect to a given HBsAg input. This test does not allow direct comparison of GA1xFlag-M samples to GA3xFlag-M ones, as M2 antibody affinity for 3xFlag-M is assumed to be higher than for 1xFlag-M. In other words, at comparable HBsAg input and OD₄₅₀ₙₘ. output, more fusion protein is present in the GA1xFlag-M sample when compared to an equivalent GA3xFlag-M sample.

### EXAMPLE 3

### Expression Vectors and Constructions

To obtain the pGA1xFlag-MpoI1.A2, pGA1xFlag-MpoI2.A2, pGA3xFlag-MpoI1.A2 and pGA3xFlag-MpoI2.A2 plasmids (FIGS. 10, 11, 12, and 13, respectively), the poI1.A2, poI2.A2 polyepitopes were cloned between the EcoRI and XhoI restriction sites of the pGA1xFlag-M and pGA3xFlag-M plasmids, respectively. Codon usage was optimized according to the Homo sapiens table (http://www.kazusa.or.ip/codon). Hydrophathy profiles were obtained by DNA StriderTM 1.2 (Kyte-Doolittle option).

The poI1.A2, poI2.A2 polyepitopes were assembled by multiple rounds of “atypical PCR.” Briefly, a series of four 60-80-mer oligonucleotides were synthesized corresponding to the plus strand of each polyepitope and overlapped one another by ˜20 bases at both 5′ and 3′ ends (the oligonucleotides used in this invention are shown in Table 3.

TABLE 3

Oligonucleotides used for pol1.A2 and pol2.A2
polyepitopes construction

| Oligonucleotide | Sequence |
|---|---|
| pol1.A2-1 | 5′-attcGTGCTGGATGTGGGAGATGCCTACTTC |
| | TCAGTGAGAGCTGACACCTACCTGAATGCCTGGG |
| | TGAAGGTG-3′ |
| pol1.A2-2 | 5′-ACCTGAATGCCTGGGTGAAGGTGGTCAGAGC |
| | CAAGACCTACCTGGTGAAGCTGTGGTACCAGCTG |
| | AGGACAG-3′ |
| pol1.A2-3 | 5′-AGCTGTGGTACCAGCTGAGGACAGATGCCTC |
| | CCTGGTGAAGCATCACATGTATGTGAGAGACACA |
| | G-3′ |
| pol1.A2-4 | 5′-AGCATCACATGTATGTGAGAGACACAGCCTA |
| | CATCTACCAGTACATGGATGACCTGAG-3′ |
| 5′ pol1.A2 | 5′-GAGAATgaattcGTGCTGGATGTGGGAGAT |
| | G-3′ |
| 3′ pol1.A2 | 5′-CTATATctcgagTCTCAGGTCATCCATGTAC |
| | TGGTAG-3′ |
| pol2.A2-1 | 5′-attcCTGCTTGACACAGGAGCTGATGACACA |
| | GTGAGGACAGATGCCAGCCTGTATAACACAGTGG |
| | CCACCCTG-3′ |
| pol2.A2-2 | 5′-AGCCTGTATAACACAGAGGCCACCCTGAGAG |
| | CTGACACCTACCTGAAGGAGCCTGTGCATGGAGT |
| | GAGAG-3′ |
| pol2.A2-3 | 5′-AGCCTGTGCATGGAGTGAGAGCTAAGACCCT |
| | CCTGTGGAAGGGAGAGGGAGCAGTGAGAACCAAG |
| | GCAGTG-3′ |
| pol2.A2-4 | 5′-AGCAGTGAGAACCAAGGCAGTGCTGGCTGAG |
| | GCCATGTCCCAGGTGAGActcgag-3′ |
| 5′ pol2.A2 | 5′-GAGAATgaattcCTGCTTGAGACAGGAGCT |
| | G-3′ |
| 3′ pol2.A2 | 5′-CTAGATctgagTCTCACCTGGGACATG-3′40 |

Two separate reactions (A and B) for poI1.A2 and poI2.A2 polyepitopes, respectively, were performed using 50 pmols of poI1.A2-1, -2, -3, -4 and 10 pmols of 3′ poll .A2 in reaction A, and 50 pmols of poI2.A2-1, -2, -3, -4 and 10 pmols of 3′ poI2.A2 in B. Six cycles of PCR were then performed.

Then, 25 cycles of classical PCR were performed, adding 100 pmols of the 5′ poI1.A2 and 3′ poI1.A2 for reactions A, and 5′ poI2.A2 and 3′ poI2.A2 for reaction B.

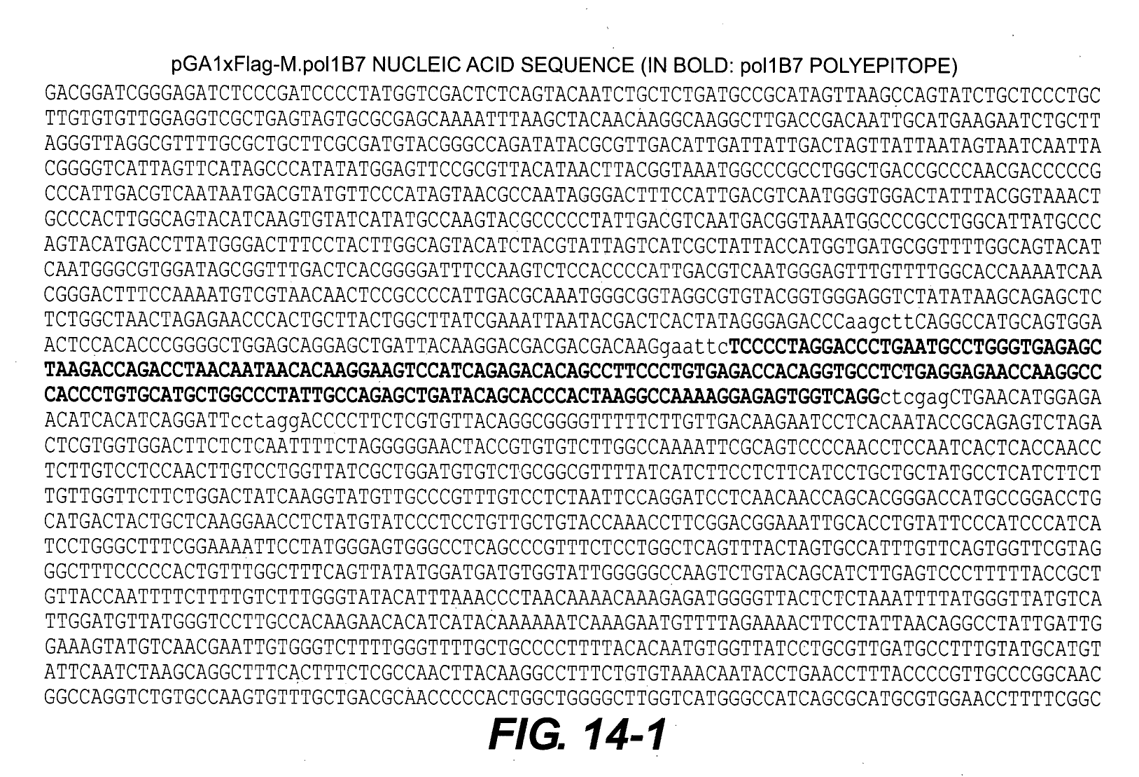

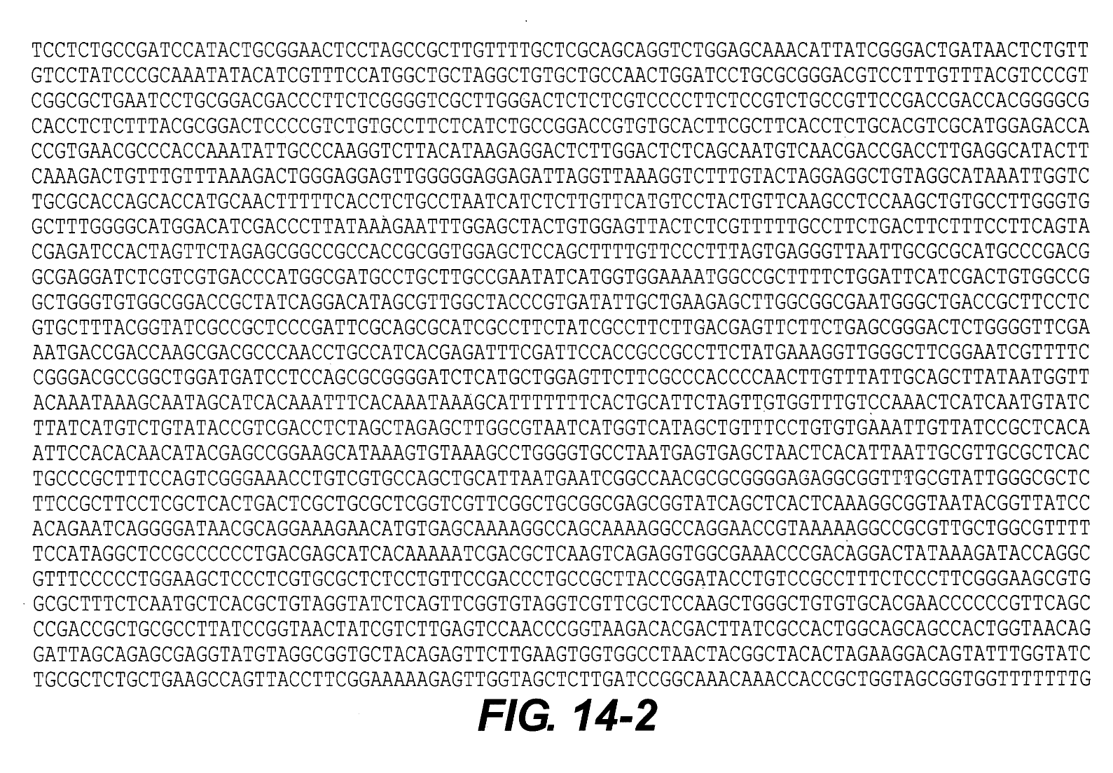

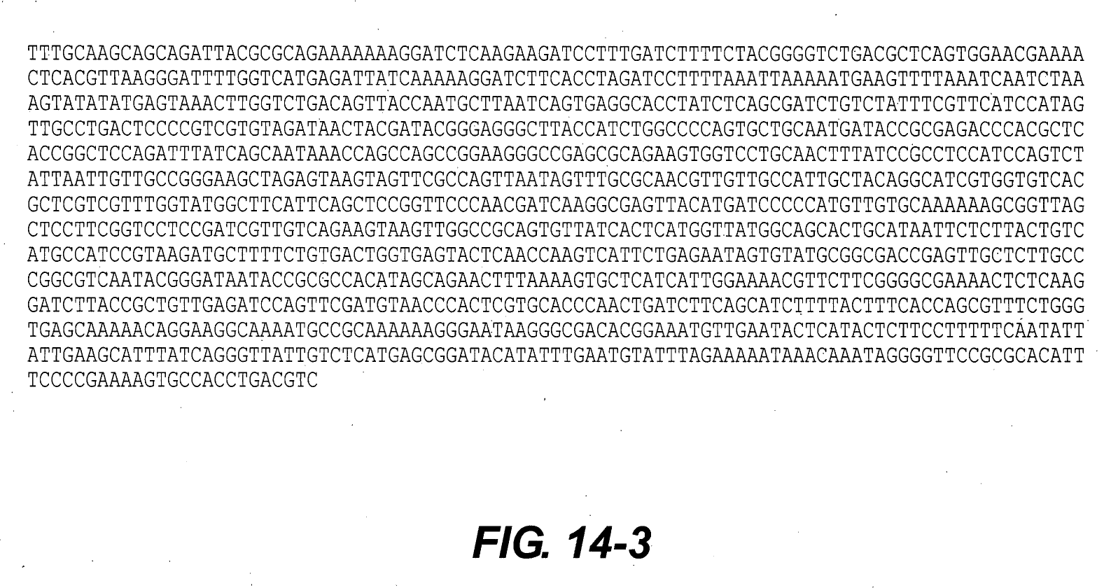

To obtain the pGA1xFlag-MpoI1.B7, pGA1xFlag-MpoI2.B7, pGA3xFlag-MpoI1B7 and pGA3xFlag-MpoI2B7 plasmids (FIGS. 14, 15, 16, and 17, respectively), the poI1.B7, poI2.B7 polyepitopes were cloned between the EcoRI and XhoI restriction sites of the pGA1xFlag-M and pGA3xFlag-M plasmids, respectively. Codon usage was optimized according to the Homo sapiens table (http://www kazusa.or.ip/capon). Hydrophathy profiles were obtained by DNA StriderTM 1.2 (Kyte-Doolittle option).

The poI1.B7, poI2.B7 polyepitopes were assembled by multiple rounds of “atypical PCR.” Briefly, a series of four 60-80-mer oligonucleotides were synthesized corresponding to the plus strand of each polyepitope and overlapped one another by ˜20 bases at both 5′ and 3′ ends (The oligonucleotides used in this invention are shown in Table 4).

More particularly, the poI1.B7, poI2.B7 polyepitopes were synthesized by multiple rounds of “atypical” PCR using the long primers detailed in the Table 4:

TABLE 4

Oligonucleotides used for pol1.B7 and pol2.B7
polyepitopes construction

| | |
|---|---|
| Oligonucleotide | Sequence |

| | |
|---|---|
| pol1.HIVB7-1 | 5′-AATTCCCCTAGGACCCTGAATGGCTGGGTGA |
| | GAGCTAAGACCAGACCTAACAATAACACAAGGAA |
| | G-3′ |
| pol1.HIVB7-2 | 5′-ACCAGACCTAACAATAACACAAGGAAGTCCA |
| | TCAGAGACACAGCCTTCCCTGTGAGACCACAGGT |
| | GCCTCTGAG-3′ |
| pol1.HIVB7-3 | 5′-AGACCACAGGTGCCTCTGAGGAGAACCAAGG |
| | CCCACCCTGTGCATGCTGGCCCTATTGCCAGAGC |
| | TG-3′ |
| pol1.HIVB7-4 | 5′-ATGCTGGCCCTATTGCCAGAGCTGATACAGC |
| | ACCCACTAAGGCCAAAAGGAGAGTGGTCAGGCTC |
| | GAG-3′ |
| 5′ pol1.HIVB7 | 5′-GTATAAhaattcTCCCCTAGGACCCTGAATG |
| | CCTG-3′ |
| 3′ pol1.HIVB7 | 5′-CTATATGCTCGAGCCTGACCACTCTCCTTTT |
| | G-3′ |
| pol2.HIVB7-1 | 5′-AATTCCAAGCCTGTGGTCTCCACACAGCTGC |
| | TTCTCAGGGCCAAGACCTTCCCTGTGAGACCCCA |
| | AGTG-3′ |
| pol2.HIVB7-2 | 5′-ACCTTCCCTGTGAGACCCCAAGTGCCACTGA |
| | GAAGGGCTGATACACAGCCCAGGAGTGACACCCA |
| | TGTGTTCAG-3′ |
| pol2.HIVB7-3 | 5′-AGGAGTGACACCCATGTGTTCAGAACCAAGG |
| | CCATTCCTAGGAGAATTAGGCAGGGCCTGAGAGA |
| | TACAG-3′ |
| pol2.HIVB7-4 | 5′-ATTAGGCAGGGCCTGAGAGATACAGCTACAC |
| | CTCAGGACCTGAACACCATGCTGAGACTCGA |
| | G-3′ |
| 5′ pol2.HIVB7 | 5′-GTCCTgaatcAAGCCTGTGGTCTCCACACA |
| | G-3′ |
| 3′ pol2.HIVB7 | 5′-CTATATGCTCGAGTCTCAGCATGGTGTTCA |
| | G-3′40 |

Two separate reactions (A and B), for poI1.B7 and poI2.B7 polyepitopes, respectively, were performed using 50 pmols of poI1.HIVB7-1, -2, -3, -4, and 10 pmols of 3′poI1.HIVB7 in reaction A, and 50 pmols of poI2.HIVB7-1, -2, -3, -4, and 10 pmols of 3′ poI2.HIVB7 in B. Six cycles of PCR were then performed.

Then, 25 cycles of classical PCR were performed, adding 100 pmols of the 5′ poll.HIVB7 and 3′ poI1.HIVB7 for reactions A, and 5′ poI2.HIVB7 and 3′ poI2.HIVB7 for reaction B.

### EXAMPLE 4

### In Vitro Evaluation of VLPs Secretion by Plasmids Bearing the poI1.A2.poI2A2, poI1B7 and poI2.B7 polyepitopes

pGA1xFlag-MpoI1.A2, pGA1xFlag-MpoI2.A2, pGA3xFlag-MpoI1.A2, pGA3xFlag-MpoI2.A2, pGA1xFlag-MpoI1.B7, pGA1xFlag-MpoI2.B7, pGA3xFlag-MpoI1.B7 and pGA3xFlag-MpoI2.B7 plasmids were transiently transfected into SW480 cells, along with pCMV-S2.S as positive controls for HBsAg VLPs formation and secretion (FIGS. 18, 19, 20, and 21). The pCMV-S2.S plasmid expresses the wild type preS2 HBsAg fusion proteins (23)

More particularly, the SW480 human cell line was maintained in Dulbecco medium supplemented with 5% foetal calf serum (FCS) and 1% streptomycin and penicillin, according to recommendations of the manufacturer. The pCMV-S2.S plasmid was kindly provided by Dr. Marie-Louise Michel (23).

Cells were transiently transfected by FuGENE6™ transfection reagent (Roche). Out of 2 ml, 500 μl of supernatant were collected and renewed at each time point. HBsAg concentration in supernatants was estimated by the Monolisa® Ag HBsAg Plus Kit (BIORAD).

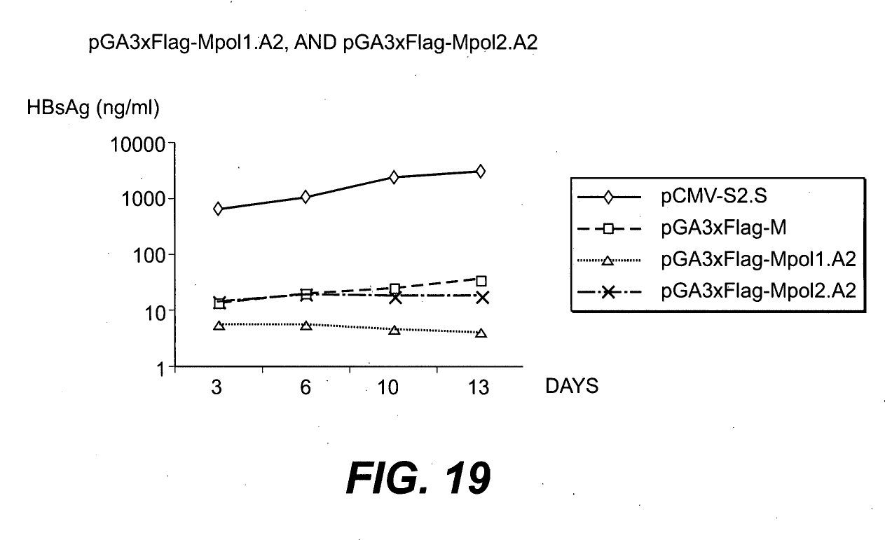

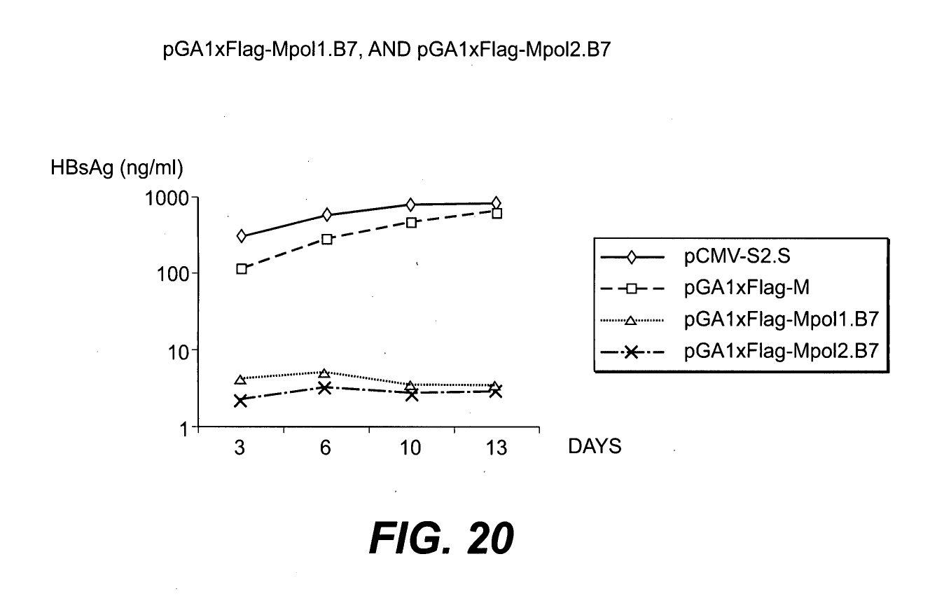

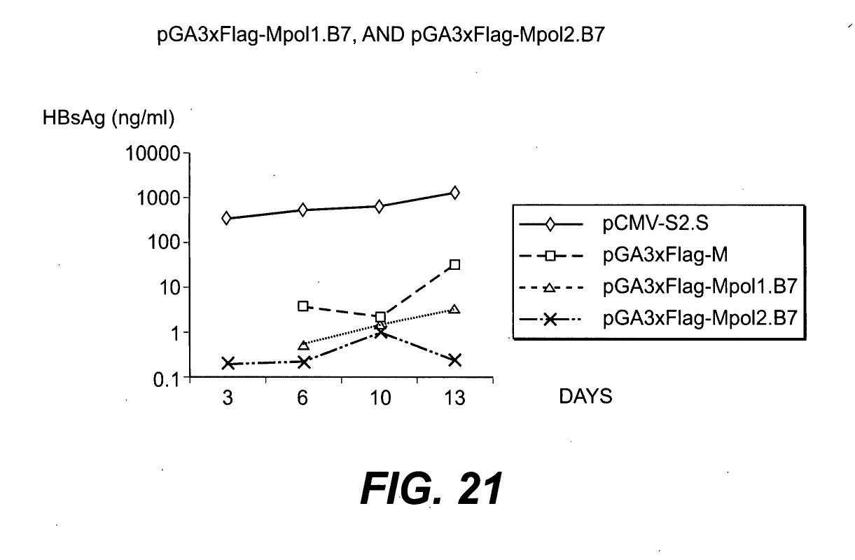

The ELISA test used allows detection and quantification of HBsAg antigenic units only if the protein is assembled into VLPs. pGA1xFlag-MpoI1.A2, pGA1xFlag-MpoI2.A2, pGA3xFlag-MpoI1.A2, pGA3xFlag-MpoI2.A2 plasmids resulted in VLPs secretion (FIGS. 18 and 19) when compared to pGA1xFlag-MpoI1.B7, pGA1xFlag-MpoI2.A2B7 pGA3xFlag-MpoI1.B7, pGA3xFlag-MpoI2.B7 plasmids (FIGS. 20 and 21). This is in keeping with the fact that HLA.A2.1 epitopes are generally more hydrophilic than the HLA.B7 ones. Moreover, the pGA1xFlag-MpoI1.A2, pGA1xFlag-MpoI2.A2, pGA3xFlag-MpoI1.A2, pGA3xFlag-MpoI2.A2 plasmids resulted in VLPs production comparable to that obtained by pGA1xFlag-MpoI.opt, pGA3xFlag-MpoI.opt (FIG. 8), all these constructions sharing HLA.A2.1 restricted epitopes.

Thus, this invention provides the GA1xFlag-M nucleotide sequence (5′->3′):

CAGGCCATGCAGTGGAACTCCACAcccgggGCTGGAGCAGGAGCTGATTA
CAAGGACGACGACGACAAGgaattcCTGCAGGCTAGCAGATCTctcgagC
TGAACATG.

The GA1xFlag-M nucleotide sequence can be comprised of the following elements:

1) CAGGCC corresponding to preS1 C-terminal sequence of HBV strain U95551 (nucleotides 3168-3173),

2) ATGCAGTGGAACTCCACA corresponding to preS2 N-terminal sequence of HBV strain U95551 (nucleotides 3174-3182; 1-9). Note: the T nucleotide in HBV strain U95551 position 3 has been substituted by C in the present invention,

3) cccggg is the SmaI restriction site (overlapping the following motif),

4) gggGCTGGAGCAGGAGCT encoding the GAGAGA amino acid sequence of the spacer motif,

5) GATTACAAGGACGACGACGACMG corresponding to the 1xFlag-M nucleotide sequence,

6) gaaftcCTGCAGGCTAGCAGATCTctcgag corresponding to EcoRI, PstI, NheI, BgII, and XhoI polylinker,

7) CTGAAC corresponding to preS2 C-terminal sequence of HBV strain U95551 (nucleotides 151-156), and

8) ATG is the first ATG codon of any S HBV protein.

This invention also provides the GA3xFlag-M nucleotide sequence (5′->3′):

CAGGCCATGCAGTGGAACTCCACAcccgggGCTGGAGCAGGAGCTGACTA
CAAAGACCACGACGGTGATTATAAAGATCACGACATTGATTACAAGGACG
ACGACGACAAGgaattcCTGCAGGCTAGCAGATCTctcgagCTGAACATG

The GA3xFlag-M nucleotide sequence can be comprised of the following elements:

1) CAGGCC corresponding to preS1 C-terminal sequence of HBV strain U95551 (nucleotides 3168-3173),

2) ATGCAGTGGMCTCCACA corresponding to preS2 N-terminal sequence of HBV strain U95561 (nucleotides 3174-3182; 1-9). Note: the T nucleotide in HBV strain U95551 position 3 has been substituted by C in the present invention,

3) cccggg corresponding to the SmaI restriction site (overlapping the following motif),

4) gggGCTGGAGCAGGAGCT encoding GAGAGA amino acid sequence of the spacer motif,

5) GACTACMAGACCACGACGGTGATTATAAAGATCACGAC ATTGATTACM GGACGACGACGACMG corresponding to 3xFlag-M nucleotide sequence,

6) gaattcCTGCAGGCTAGCAGATCTctcgag corresponding to EcoRI, PstI, NheI, BgII, and XhoI polylinker,

7) CTGAAC corresponding to preS2 C-terminal sequence of HBV strain U95551 (nucleotides 151-156), and

8) ATG is the first ATG codon of any S HBV protein.

E. coli strains carrying the following plasmids were deposited at the Collection Nationale de Cultures de Microorganismes (C.N.C.M.), of Institut Pasteur, 25, rue du Docteur Roux, F-75724 Paris, Cedex 15, France, and assigned the following Accession Nos.:

| Plasmid | Accession No. |
|---|---|
| pGA1xFlag-M | CNCM I-3543 filed on Dec. 16, 2005 |
| pGA1xFlag-Mpol.opt | CNCM I-3544 filed on Dec. 16, 2005 |
| pGA3xFlag-M | CNCM I-3545 filed on Dec. 16, 2005 |
| pGA3xFlag-Mpol.opt | CNCM I-3546 filed on Dec. 16, 2005 |
| pGA1xFlag-M.pol.1A2 | CNCM I-3579 filed on Feb. 28, 2006 |
| pGA1xFlag-M.pol.2A2 | CNCM I-3580 filed on Feb. 28, 2006 |
| pGA1xFlag-M.pol.1B7 | CNCM I-3581 filed on Feb. 28, 2006 |
| pGA1xFlag-M.pol.2B7 | CNCM I-3582 filed on Feb. 28, 2006. |

### REFERENCES

The following references are cited herein. The entire disclosure of each reference is relied upon and incorporated by reference herein.
1. Albert, M. L., S. F. Pearce, L. M. Francisco, B. Sauter, P. Roy, R. L. Silverstein, and N. Bhardwaj. 1998. Immature dendritic cells phagocytose apoptotic cells via alphavbeta5 and CD36, and cross-present antigens to cytotoxic T lymphocytes. J. Exp. Med 188:1359-1368.
2. Bisht, H., D. A. Chugh, M. Raje, S. S. Swaminathan, and N. Khanna. 2002. Recombinant dengue virus type 2 envelope/hepatitis B surface antigen hybrid protein expressed in Pichia pastoris can function as a bivalent immunogen. J. Biotechnol. 99:97-110.
3. Boisgerault, F., G. Moron, and C. Leclerc. 2002. Virus-like particles: a new family of delivery systems. Expert Rev. Vaccines 1:101-109.
4. Boyle, J. S., C. Koniaras, and A. M. Lew. 1997. Influence of cellular location of expressed antigen on the efficacy of DNA vaccination: cytotoxic T lymphocyte and antibody responses are suboptimal when antigen is cytoplasmic after intramuscular DNA immunization. Int. Immunol. 9:1897-1906.
5. Bruss, V. 2004. Envelopment of the hepatitis B virus nucleocapsid. Virus Res. 106:199-209.
6. Bullock, T. N., T. A. Colella, and V. H. Engelhard. 2000. The density of peptides displayed by dendritic cells affects immune responses to human tyrosinase and gp100 in HLA-A2 transgenic mice. J. Immunol. 164:2354-2361.
7. Buseyne, F., M. Fevrier, S. Garcia, M. L. Gougeon, and Y. Riviere. 1996. Dual function of a human immunodeficiency virus (HIV)-specific cytotoxic T-lymphocyte clone: inhibition of HIV replication by noncytolytic mechanisms and lysis of HIV-infected CD4+ cells. Virology 225:248-253.30
8. Chua, P. K., R. Y. Wang, M. H. Lin, T. Masuda, F. M. Suk, and C. Shih. 2005. Reduced secretion of virions and hepatitis B virus (HBV) surface antigen of a naturally occurring HBV variant correlates with the accumulation of the small s envelope protein in the endoplasmic reticulum and Golgi apparatus. J. Virol. 79:13483-13496.
9. Davis, H. L., C. L. Millan, and S. C. Watkins. 1997. Immune-mediated destruction of transfected muscle fibers after direct gene transfer with antigen-expressing plasmid DNA. Gene Ther. 4:181-188.
10. Doan, L. X., M. Li, C. Chen, and Q. Yao. 2005. Virus-like particles as HIV-1 vaccines. Rev. Med. Virol. 15:75-88.
11. Firat, H., F. Garcia-Pons, S. Tourdot, S. Pascolo, A. Scardino, Z. Garcia, M. L. Michel, R. W. Jack, G. Jung, K. Kosmatopoulos, L. Mateo, A. Suhrbier, F. A. Lemonnier, and P. Langlade-Demoyen. 1999. H-2 class I knockout, HLA-A2.1-transgenic mice: a versatile animal model for preclinical evaluation of antitumor immunotherapeutic strategies. Eur. J. Immunol. 29:3112-3121.
12. Firat, H., S. Tourdot, A. Ureta-Vidal, A. Scardino, A. Suhrbier, F. Buseyne, Y. Riviere, O. Danos, M. L. Michel, K. Kosmatopoulos, and F. A. Lemonnier. 2001. Design of a polyepitope construct for the induction of HLA-A0201-restricted HIV 1-specific CTL responses using HLA-A*0201 transgenic, H-2 class I KO mice. Eur. J. Immunol. 31:3064-3074.
13. Inaba, K., S. Turley, F. Yamaide, T. lyoda, K. Mahnke, M. Inaba, M. Pack, M. Subklewe, B. Sauter, D. Sheff, M. Albert, N. Bhardwaj, I. Mellman, and R. M. Steinman. 1998. Efficient presentation of phagocytosed cellular fragments on the major histocompatibility complex class II products of dendritic cells. J. Exp. Med. 188:2163-2173.
14. Kozak, M. 2002. Pushing the limits of the scanning mechanism for initiation of translation. Gene 299:1-34.
15. Kypr, J., and J. Mrazek. 1987. Unusual codon usage of HIV. Nature 327:20.
16. Le Borgne, S., M. Mancini, R. Le Grand, M. Schleef, D. Dormont, P. Tiollais, Y. Riviere, and M. L. Michel. 1998. In vivo induction of specific cytotoxic T lymphocytes in mice and rhesus macaques immunized with DNA vector encoding an HIV epitope fused with hepatitis B surface antigen. Virology 240:304-315.
17. Lenz, P., C. D. Thompson, P. M. Day, S. M. Bacot, D. R. Lowy, and J. T. Schiller. 2003. Interaction of papillomavirus virus-like particles with human myeloid antigenpresenting cells. Clin. Immunol. 106:231-237.
18. Lewis, P. J., H. van Drunen Littel-van den, and L. A. Babiuk. 1999. Altering the cellular location of an antigen expressed by a DNA-based vaccine modulates the immune response. J. Virol. 73:10214-10223.
19. Li, H. Z., H. Y. Gang, Q. M. Sun, X. Liu, Y. B. Ma, M. S. Sun, and C. B. Dai. 2004. Production in Pichia pastoris and characterization of genetic engineered chimeric HBV/HEV virus-like particles. Chin. Med. Sci. J. 19:78-83.
20. Livingston, B. D., M. Newman, C. Crimi, D. McKinney, R. Chesnut, and A. Sette. 2001. Optimization of epitope processing enhances immunogenicity of multiepitope DNA vaccines. Vaccine 19:4652-4660.
21. Marsac, D., A.-L. Puaux, Y. Riviere, and M. L. Michel. 2005. In vivo induction of cellular and humoral immune response by hybrid DNA vectors encoding simian/human immunodeficiency virus/hepatitis B surface antigen virus particules in BALB/c and HLA-A2-transgenic mice. Immunobiology 210:305-319.
22. Mathet, V. L., M. Feld, L. Espinola, D. O. Sanchez, V. Ruiz, O. Mando, G. Carballal, J. F. Quarleri, F. D'Mello, C. R. Howard, and J. R. Oubina. 2003. Hepatitis B virus S gene mutants in a patient with chronic active hepatitis with circulating Anti-HBs antibodies. J Med Virol 69:18-26.
23. Michel, M. L., H. L. Davis, M. Schleef, M. Mancini, P. Tiollais, and R. G. Whalen. 1995. DNA-mediated immunization to the hepatitis B surface antigen in mice: aspects of the humoral response mimic hepatitis B viral infection in humans. Proc. Natl. Acad. Sci. USA 92:5307-5311.
24. Michel, M. L., and D. Loirat. 2001. DNA vaccines for prophylactic or therapeutic immunization against hepatitis B. Intervirology 44:78-87.
25. Morel, P. A., D. Falkner, J. Plowey, A. T. Larregina, and L. D. Falo. 2004. DNA immunization: altering the cellular localisation of expressed protein and the immunization route allows manipulation of the immune response. Vaccine 22:447-456.
26. Pajot, A., M. L. Michel, N. Fazilleau, V. Pancre, C. Auriault, D. M. Ojcius, F. A. Lemonnier, and Y. C. Lone. 2004. A mouse model of human adaptive immune functions: HLA-A2.1-/HLA-DR1-transgenic H-2 class I-/class II-knockout mice. Eur. J. Immunol. 34:3060-3069.
27. Pajot, A., V. Pancre, N. Fazilleau, M. L. Michel, G. Angyalosi, D. M. Ojcius, C. Auriault, F. A. Lemonnier, and Y. C. Lone. 2004. Comparison of HLA-DR1-restricted T cell response induced in HLA-DR1 transgenic mice deficient for murine MHC class II and HLA-DR1 transgenic mice expressing endogenous murine MHC class II molecules. Int. Immunol. 16:1275-1282.
28. Pascolo, S., N. Bervas, J. M. Ure, A. G. Smith, F. A. Lemonnier, and B. Perarnau. 1997. HLA-A2.1-restricted education and cytolytic activity of CD8(+) T lymphocytes from beta2 microglobulin (beta2m) HLA-A2.1 monochain transgenic H-2Db beta2m double knockout mice. J. Exp. Med. 185:2043-2051.
29. Peixoto, L., A. Zavala, H. Romero, and H. Musto. 2003. The strength of translational selection for codon usage varies in the three replicons of Sinorhizobium meliloti. Gene 320:109-116.
30. Pumpens, P., R. Razanskas, P. Pushko, R. Renhof, I. Gusars, D. Skrastina, V. Ose, G. Borisova, I. Sominskaya, I. Petrovskis, J. Jansons, and K. Sasnauskas. 2002. Evaluation of HBs, HBc, and frCP virus-like particles for expression of human papillomavirus 16 E7 oncoprotein epitopes. Intervirology 45:24-32.
31. Rajcani, J., T. Mosko, and 1. Rezuchova. 2005. Current developments in viral DNA vaccines: shall they solve the unsolved? Rev. Med. Virol. 15:303-325.
32. Romero, H., A. Zavala, H. Musto, and G. Bernardi. 2003. The influence of translational selection on codon usage in fishes from the family Cyprinidae. Gene 317:141-147.
33. Rudolf, M. P., S. C. Fausch, D. M. Da Silva, and W. M. Kast. 2001. Human dendritic cells are activated by chimeric human papillomavirus type-16 virus-like particles and induce epitope-specific human T cell responses in vitro. J. Immunol. 166:5917-5924.
34. Rudolf, M. P., J. D. Nieland, D. M. DaSilva, M. P. Velders, M. Muller, H. L. Greenstone, J. T. Schiller, and W. M. Kast. 1999. Induction of HPV16 capsid protein-specific human T cell responses by virus-like particles. Biol. Chem. 380:335-340.
35. Ruedl, C., T. Storni, F. Lechner, T. Bachi, and M. F. Bachmann. 2002. Cross-presentation of virus-like particles by skin-derived CD8(−) dendritic cells: a dispensable role for TAP. Eur. J. Immunol. 32:818-825.
36. Schreckenberger, C., and A. M. Kaufmann. 2004. Vaccination strategies for the treatment and prevention of cervical cancer. Curr. Opin. Oncol. 16:485-491.
37. Stern, P. L. 2005. Immune control of human papillomavirus (HPV) associated anogenital disease and potential for vaccination. J. Clin. Virol. 32 Suppl 1:S72-81.
38. Subklewe, M., C. Paludan, M. L. Tsang, K. Mahnke, R. M. Steinman, and C. Munz. 2001. Dendritic cells cross-present latency gene products from Epstein-Barr virus-transformed B cells and expand tumor-reactive CD8(+) killer T cells. J. Exp. Med. 193:405-411.
39. Wang, S., D. J. Farfan-Arribas, S. Shen, T. H. Chou, A. Hirsch, F. He, and S. Lu. 2005. Relative contributions of codon usage, promoter efficiency and leader sequence to the antigen expression and immunogenicity of HIV-1 Env DNA vaccine. Vaccine. In Press.
40. Wang, Y., J. A. Smith, T. Kamradt, M. L. Gefter, and D. L. Perkins. 1992. Silencing of immunodominant epitopes by contiguous sequences in complex synthetic peptides. Cell. Immunol. 143:284-297.
41. Wherry, E. J., M. J. McElhaugh, and L. C. Eisenlohr. 2002. Generation of CD8(+) T cell memory in response to low, high, and excessive levels of epitope. J. Immunol. 168:4455-4461.
42. Yan, M., J. Peng, I. A. Jabbar, X. Liu, L. Filgueira, I. H. Frazer, and R. Thomas. 2004. Despite differences between dendritic cells and Langerhans cells in the mechanism of papillomavirus-like particle antigen uptake, both cells cross-prime T cells. Virology 324:297-310.

What is claimed is:

1. A polynucleotide comprising GA1xFlag-M polynucleotide of sequence:
[SEQ ID NO: 1]
CAGGCCATGCAGTGGAACTCCACAcccgggGCTGGAGCAGGAGCT
GATTACAAGGACGACGACGACAAGgaattcCTGCAGGCTAGCAGA
TCTctcgagCTGAACATG;
or GA3XFlag-M polynucleotide of sequence:
[SEQ ID NO: 2]
CAGGCCATGCAGTGGAACTCCACAcccgggGCTGGAGCAGGAGCT
GACTACAAAGACCACGACGGTGATTATAAAGATCACGACATTGAT
TACAAGGACGACGACGACAAGgaattcCTGCAGGCTAGCAGATCT
ctcgagCTGAACATG.

2. A polynucleotide as claimed in claim 1, which comprises an eukaryotic promoter operably linked to the GA1xFlag-M polynucleotide or the GA3xFlag-M polynucleotide.

3. A polynucleotide as claimed in claim 2, which comprises a nucleotide sequence encoding hepatitis B surface antigen protein (HBsAg) devoid of translation initiation ATG and positioned downstream and in frame with the GA1xFlag-M or the GA3xFlag-M polynucleotide sequence.

4. A polynucleotide as claimed in claim 2 or 3, which comprises a polyadenylation sequence operably linked to the other sequences.

5. A polynucleotide as claimed in claim 1 comprising the polynucleotide sequence cloned between HindIII and AvrII restriction sites in pGA1xFlag-M plasmid deposited at the CNCM on Dec. 16, 2005, under the Accession Number 1-3543.

6. A polynucleotide as claimed in claim 1 comprising the polynucleotide cloned between HindIII and AvrII restriction sites in pGA3xFlag-M plasmid deposited at the CNCM on Dec. 16, 2005, under the Accession Number 1-3545.

7. A polynucleotide as claimed in claim 1 comprising the GA1xFlag-M polynucleotide or the GA3xFlag-M polynucleotide, a eukaryotic promoter sequence, a nucleotide sequence encoding hepatitis B surface antigen protein (HBsAg), and a polyadenylation sequence.

8. A polynucleotide hybridizing under stringent conditions to the polynucleotide as claimed in claim 1 or its complement.

9. A polynucleotide as claimed in any of claims 1 to 10, wherein it further comprises a foreign coding polynucleotide inserted in any of restriction sites of the GA1xFlag-M or GA3xFlag-M polynucleotide and in frame with the ATG at position 7 in the GA1xFlag-M or GA3xFlag-M polynucleotide sequence.

10. A cloning and/or expression vector comprising a polynucleotide as claimed in any one of claims 1-9.

11. A eukaryotic host cell comprising a vector as claimed in claim 10.

12. A eukaryotic host cell as claimed in claim 11, wherein the vector comprises an eukaryotic promoter sequence operably linked to a nucleotide sequence encoding HBsAg protein for expression of HBsAg virus-like particles.

13. A eukaryotic host cell as claimed in claim 12, wherein the vector comprises a nucleotide sequence encoding a HBsAg fusion protein comprising a foreign polypeptide and HBsAg protein, and wherein the eukaryotic host cell produces HBsAg virus-like particles constituted by said HBsAg fusion protein and HBsAg protein.

14. A method of producing HBsAg virus-like particles, wherein the method comprises:
providing a host cell as claimed in any one of claims 11-13; and
expressing the fusion proteins and HBsAg proteins under conditions in which the proteins assemble into virus-like particles, which are released from the host cell into extracellular space.

15. A method as claimed in claim 14, which comprises recovering the virus-like particles.

16. A method of preparing a HBsAg fusion protein, wherein the method comprises:
providing a host cell as claimed in any one of claims 11 to 13;
expressing a tagged HBsAg fusion protein and HbsAg protein under conditions in which the proteins assemble into virus-like particles, which are released from the host cell into extracellular space; and
separating the VLP bearing tagged HBsAg fusion proteins from the bacteria culture by capture with Flag-M antibodies and/or HBsAg antibodies.

17. An expression vector as claimed in claim 10, wherein it is selected from pGA1xFlag-M (CNCM No. I-3543), pGA3xFlag-M (CNCM No. I-3545), pGA1xFlag-M poI.opt (CNCM No. I-3544), pGA3xFlag-M poI.opt (CNCM No. I-3546), pGA1xFlag-M.poI1A2 (CNCM No. I-3579), pGA1xFlag-M.poI2A2 (CNCM No. I-3580), pGA1xFlag-M.poI1B7 (CNCM No. I-3581), and pGA1xFlag-M.poI2B7 (CNCM No. I-3582).

18. A polynucleotide comprising the sequence between HindIII and AvrII restriction sites of an expression vector as claimed in claim 17.

19. A polypeptide encoded by a polynucleotide as claimed in any of claims 1 to 9 or by a vector according to claim 10.


SEQUENCE LISTING (136 sequences)

SEQ ID NO: 1 - DNA, 108 bp, Artificial Sequence
  Description of Artificial Sequence Synthetic polynucleotide
caggccatgcagtggaactccacacccggggctggagcaggagctgattacaaggacgacgacgacaaggaattcctgcaggctagcagatctctcgagctgaacatg

SEQ ID NO: 2 - DNA, 150 bp, Artificial Sequence
  Description of Artificial Sequence Synthetic polynucleotide
caggccatgcagtggaactccacacccggggctggagcaggagctgactacaaagaccacgacggtgattataaagatcacgacattgattacaaggacgacgacgacaaggaattcctgcaggctagcagatctctcgagctgaacatg

SEQ ID NO: 3 - protein, 104 aa, Human immunodeficiency virus
YLKEPVHGVRAKTYLNAWVKVVRDTAVLDVGDAYFSVRAKTYLVKLWYQLRADTRLYNTVATLRTKALLDTGADDTVRAKTLLWKGEGAVRTDAYIYQYMDDLR

SEQ ID NO: 4 - DNA, 31 bp, Artificial Sequence
  Description of Artificial Sequence Synthetic oligonucleotide
gaattcagctagcgatatccccgggctcgag

SEQ ID NO: 5 - DNA, 140 bp, Artificial Sequence
  Description of Artificial Sequence Synthetic polynucleotide
aagcttcaggccatgcagtggaactccacacccggggctggagcaggagctgattacaaggacgacgacgacaaggaattcctgcaggctagcagatctctcgagctgaacatggagaacatcacatcaggattcctagg

SEQ ID NO: 6 - DNA, 182 bp, Artificial Sequence
  Description of Artificial Sequence Synthetic polynucleotide
aagcttcaggccatgcagtggaactccacacccggggctggagcaggagctgactacaaagaccacgacggtgattataaagatcacgacattgattacaaggacgacgacgacaaggaattcctgcaggctagcagatctctcgagctgaacatggagaacatcacatcaggattcctagg

SEQ ID NO: 7 - protein, 7 aa, Hepatitis B virus
MQWNSTP

SEQ ID NO: 8 - protein, 6 aa, Artificial Sequence
  Description of Artificial Sequence Synthetic peptide
GAGAGA

SEQ ID NO: 9 - protein, 6 aa, Artificial Sequence
  Description of Artificial Sequence Synthetic peptide
QWNSTP

SEQ ID NO: 10 - protein, 36 aa, Artificial Sequence
  Description of Artificial Sequence Synthetic peptide
QAMQWNSTPGAGAGADYKDDDDKEFLQASRSLELNM

SEQ ID NO: 11 - protein, 50 aa, Artificial Sequence
  Description of Artificial Sequence Synthetic peptide
QAMQWNSTPGAGAGADYKDHDGDYKDHDIDYKDDDDKEFLQASRSLELNM

SEQ ID NO: 12 - protein, 9 aa, Human immunodeficiency virus
SLYNTVATL

SEQ ID NO: 13 - protein, 8 aa, Human immunodeficiency virus
TLNAWVKW

SEQ ID NO: 14 - protein, 10 aa, Human immunodeficiency virus
LLDTGADDTV

SEQ ID NO: 15 - protein, 11 aa, Human immunodeficiency virus
VLDVGDAYFSV

SEQ ID NO: 16 - protein, 9 aa, Human immunodeficiency virus
VIYQYMDDL

SEQ ID NO: 17 - protein, 9 aa, Human immunodeficiency virus
ILKEPVHGV

SEQ ID NO: 18 - protein, 9 aa, Human immunodeficiency virus
PLVKLWYQL

SEQ ID NO: 19 - protein, 11 aa, Human immunodeficiency virus
ESELVNQIIEQ

SEQ ID NO: 20 - protein, 10 aa, Human immunodeficiency virus
ELVNQIIEQL

SEQ ID NO: 21 - protein, 9 aa, Human immunodeficiency virus
LLWKGEGAV

SEQ ID NO: 22 - protein, 9 aa, Human immunodeficiency virus
RLRDLLLIV

SEQ ID NO: 23 - protein, 9 aa, Human immunodeficiency virus
AFHHVAREL

SEQ ID NO: 24 - DNA, 313 bp, Artificial Sequence
  Description of Artificial Sequence Synthetic polynucleotide
ctacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttaga

SEQ ID NO: 25 - protein, 104 aa, Artificial Sequence; identical to SEQ ID NO: 3
  Description of Artificial Sequence Synthetic polypeptide

SEQ ID NO: 26 - DNA, 192 bp, Artificial Sequence
  Description of Artificial Sequence Synthetic polynucleotide
gtgctggatgtgggagatgcctacttctcagtgagagctgacacctacctgaatgcctgggtgaaggtggtcagagccaagacctacctggtgaagctgtggtaccagctgaggacagatgcctccctggtgaagcatcacatgtatgtgagagacacagcctacatctaccagtacatggatgacctgaga

SEQ ID NO: 27 - protein, 64 aa, Artificial Sequence
  Description of Artificial Sequence Synthetic peptide
VLDVGDAYFSVRADTYLNAWVKVVRAKTYLVKLWYQLRTDASLVKHHMYVRDTAYIYQYMDDLR

SEQ ID NO: 28 - protein, 11 aa, Artificial Sequence
  Description of Artificial Sequence Synthetic peptide
VLDVGDAYFSV

SEQ ID NO: 29 - protein, 9 aa, Artificial Sequence
  Description of Artificial Sequence Synthetic peptide
YLNAWVKVV

SEQ ID NO: 30 - protein, 9 aa, Artificial Sequence
  Description of Artificial Sequence Synthetic peptide
YLVKLWYQL

SEQ ID NO: 31 - protein, 9 aa, Artificial Sequence
  Description of Artificial Sequence Synthetic peptide
SLVKHHMYV

SEQ ID NO: 32 - protein, 9 aa, Artificial Sequence
  Description of Artificial Sequence Synthetic peptide
YIYQYMDDL

SEQ ID NO: 33 - DNA, 33 bp, Artificial Sequence
  Description of Artificial Sequence Synthetic oligonucleotide
gtgctggatgtgggagatgcctacttctcagtg

SEQ ID NO: 34 - DNA, 27 bp, Artificial Sequence
  Description of Artificial Sequence Synthetic oligonucleotide
tacctgaatgcctgggtgaaggtggtc

SEQ ID NO: 35 - DNA, 27 bp, Artificial Sequence
  Description of Artificial Sequence Synthetic oligonucleotide
tacctggtgaagctgtggtaccagctg

SEQ ID NO: 36 - DNA, 27 bp, Artificial Sequence
  Description of Artificial Sequence Synthetic oligonucleotide
tccctggtgaagcatcacatgtatgtg

SEQ ID NO: 37 - DNA, 27 bp, Artificial Sequence
  Description of Artificial Sequence Synthetic oligonucleotide
tacatctaccagtacatggatgacctg

SEQ ID NO: 38 - DNA, 189 bp, Artificial Sequence
  Description of Artificial Sequence Synthetic polynucleotide
ctgcttgacacaggagctgatgacacagtgaggacagatgccagcctgtataacacagtggccaccctgagagctgacacctacctgaaggagcctgtgcatggagtgagagctaagaccctcctgtggaagggagagggagcagtgagaaccaaggcagtgctggctgaggccatgtcccaggtgaga

SEQ ID NO: 39 - protein, 63 aa, Artificial Sequence
  Description of Artificial Sequence Synthetic peptide
LLDTGADDTVRTDASLYNTVATLRADTYLKEPVHGVRAKTLLWKGEGAVRTKAVLAEAMSQVR

SEQ ID NO: 40 - protein, 10 aa, Artificial Sequence
  Description of Artificial Sequence Synthetic peptide
LLDTGADDTV

SEQ ID NO: 41 - protein, 9 aa, Artificial Sequence
  Description of Artificial Sequence Synthetic peptide
SLYNTVATL

SEQ ID NO: 42 - protein, 9 aa, Artificial Sequence
  Description of Artificial Sequence Synthetic peptide
YLKEPVHGV

SEQ ID NO: 43 - protein, 9 aa, Artificial Sequence
  Description of Artificial Sequence Synthetic peptide
LLWKGEGAV

SEQ ID NO: 44 - protein, 9 aa, Artificial Sequence
  Description of Artificial Sequence Synthetic peptide
VLAEAMSQV

SEQ ID NO: 45 - DNA, 30 bp, Artificial Sequence
  Description of Artificial Sequence Synthetic oligonucleotide
ctgcttgacacaggagctgatgacacagtg

SEQ ID NO: 46 - DNA, 27 bp, Artificial Sequence
  Description of Artificial Sequence Synthetic oligonucleotide
agcctgtataacacagtggccaccctg

SEQ ID NO: 47 - DNA, 27 bp, Artificial Sequence
  Description of Artificial Sequence Synthetic oligonucleotide
tacctgaaggagcctgtgcatggagtg

SEQ ID NO: 48 - DNA, 27 bp, Artificial Sequence
  Description of Artificial Sequence Synthetic oligonucleotide
ctcctgtggaagggagagggagcagtg

SEQ ID NO: 49 - DNA, 27 bp, Artificial Sequence
  Description of Artificial Sequence Synthetic oligonucleotide
gtgctggctgaggccatgtcccaggtg

SEQ ID NO: 50 - DNA, 195 bp, Artificial Sequence
  Description of Artificial Sequence Synthetic polynucleotide
tcccctaggaccctgaatgcctgggtgagagctaagaccagacctaacaataacacaaggaagtccatcagagacacagccttccctgtgagaccacaggtgcctctgaggagaaccaaggcccaccctgtgcatgctggccctattgccagagctgatacagcacccactaaggccaaaaggagagtggtcagg

SEQ ID NO: 51 - protein, 65 aa, Artificial Sequence
  Description of Artificial Sequence Synthetic peptide
SPRTLNAWVRAKTRPNNNTRKSIRDTAFPVRPQVPLRRTKAHPVHAGPIARADTAPTKAKRRVVR

SEQ ID NO: 52 - protein, 9 aa, Artificial Sequence
  Description of Artificial Sequence Synthetic peptide
SPRTLNAWV

SEQ ID NO: 53 - protein, 10 aa, Artificial Sequence
  Description of Artificial Sequence Synthetic peptide
RPNNNTRKSI

SEQ ID NO: 54 - protein, 10 aa, Artificial Sequence
  Description of Artificial Sequence Synthetic peptide
FPVRPQVPLR

SEQ ID NO: 55 - protein, 9 aa, Artificial Sequence
  Description of Artificial Sequence Synthetic peptide
HPVHAGPIA

SEQ ID NO: 56 - protein, 10 aa, Artificial Sequence
  Description of Artificial Sequence Synthetic peptide
APTKAKRRVV

SEQ ID NO: 57 - DNA, 27 bp, Artificial Sequence
  Description of Artificial Sequence Synthetic oligonucleotide
tcccctaggaccctgaatgcctgggtg

SEQ ID NO: 58 - DNA, 30 bp, Artificial Sequence
  Description of Artificial Sequence Synthetic oligonucleotide
agacctaacaataacacaaggaagtccatc

SEQ ID NO: 59 - DNA, 30 bp, Artificial Sequence
  Description of Artificial Sequence Synthetic oligonucleotide
ttccctgtgagaccacaggtgcctctgagg

SEQ ID NO: 60 - DNA, 27 bp, Artificial Sequence
  Description of Artificial Sequence Synthetic oligonucleotide
caccctgtgcatgctggccctattgcc

SEQ ID NO: 61 - DNA, 30 bp, Artificial Sequence
  Description of Artificial Sequence Synthetic oligonucleotide
gcacccactaaggccaaaaggagagtggtc

SEQ ID NO: 62 - DNA, 192 bp, Artificial Sequence
  Description of Artificial Sequence Synthetic polynucleotide
aagcctgtggtctccacacagctgcttctcagggccaagaccttccctgtgagaccccaagtgccactgagaagggctgatacacagcccaggagtgacacccatgtgttcagaaccaaggccattcctaggagaattaggcagggcctgagagatacagctacacctcaggacctgaacaccatgctgaga

SEQ ID NO: 63 - protein, 64 aa, Artificial Sequence
  Description of Artificial Sequence Synthetic peptide
KPVVSTQLLLRAKTFPVRPQVPLRRADTQPRSDTHVFRTKAIPRRIRQGLRDTATPQDLNTMLR

SEQ ID NO: 64 - protein, 10 aa, Artificial Sequence
  Description of Artificial Sequence Synthetic peptide
KPVVSTQLLL

SEQ ID NO: 65 - protein, 10 aa, Artificial Sequence
  Description of Artificial Sequence Synthetic peptide
FPVRPQVPLR

SEQ ID NO: 66 - protein, 9 aa, Artificial Sequence
  Description of Artificial Sequence Synthetic peptide
QPRSDTHVF

SEQ ID NO: 67 - protein, 9 aa, Artificial Sequence
  Description of Artificial Sequence Synthetic peptide
IPRRIRQGL

SEQ ID NO: 68 - protein, 9 aa, Artificial Sequence
  Description of Artificial Sequence Synthetic peptide
TPQDLNTML

SEQ ID NO: 69 - DNA, 30 bp, Artificial Sequence
  Description of Artificial Sequence Synthetic oligonucleotide
aagcctgtggtctccacacagctgcttctc

SEQ ID NO: 70 - DNA, 30 bp, Artificial Sequence
  Description of Artificial Sequence Synthetic oligonucleotide
ttccctgtgagaccccaagtgccactgaga

SEQ ID NO: 71 - DNA, 27 bp, Artificial Sequence
  Description of Artificial Sequence Synthetic oligonucleotide
cagcccaggagtgacacccatgtgttc

SEQ ID NO: 72 - DNA, 27 bp, Artificial Sequence
  Description of Artificial Sequence Synthetic oligonucleotide
attcctaggagaattaggcagggcctg

SEQ ID NO: 73 - DNA, 27 bp, Artificial Sequence
  Description of Artificial Sequence Synthetic oligonucleotide
acacctcaggacctgaacaccatgctg

SEQ ID NO: 74 - protein, 6 aa, Hepatitis B virus
ENITSG

SEQ ID NO: 75 - DNA, 58 bp, Artificial Sequence
  Description of Artificial Sequence Synthetic primer
caggccatgcagtggaactccacacccggggctggagcaggagctgattacaaggacg

SEQ ID NO: 76 - DNA, 65 bp, Artificial Sequence
  Description of Artificial Sequence Synthetic primer
tggagcaggagctgattacaaggacgacgacgacaaggaattcctgcaggctagcagatctctcg

SEQ ID NO: 77 - DNA, 60 bp, Artificial Sequence
  Description of Artificial Sequence Synthetic primer
tcctgcaggctagcagatctctcgagctgaacatggagaacatcacatcaggattcctag

SEQ ID NO: 78 - DNA, 55 bp, Artificial Sequence
  Description of Artificial Sequence Synthetic primer
caggccatgcagtggaactccacacccggggctggagcaggagctgactacaaag

SEQ ID NO: 79 - DNA, 64 bp, Artificial Sequence
  Description of Artificial Sequence Synthetic primer
tggagcaggagctgactacaaagaccacgacggtgattataaagatcacgacattgattacaag

SEQ ID NO: 80 - DNA, 74 bp, Artificial Sequence
  Description of Artificial Sequence Synthetic primer
tgattataaagatcacgacattgattacaaggacgacgacgacaaggaattcctgcaggctagcagatctctcg

SEQ ID NO: 81 - DNA, 57 bp, Artificial Sequence
  Description of Artificial Sequence Synthetic primer
tgcaggctagcagatctctcgagctgaacatggagaacatcacatcaggattcctag

SEQ ID NO: 82 - DNA, 36 bp, Artificial Sequence
  Description of Artificial Sequence Synthetic primer
agacccaagcttcaggccatgcagtggaactccaca

SEQ ID NO: 83 - DNA, 36 bp, Artificial Sequence
  Description of Artificial Sequence Synthetic primer
aggggtcctaggaatcctgatgtgatgttctccatg

SEQ ID NO: 84 - DNA, 71 bp, Artificial Sequence
  Description of Artificial Sequence Synthetic oligonucleotide
gaattcctacttgaaagagccagttcatggggtgagagccaagacctacctgaatgcatgggtgaaagttg

SEQ ID NO: 85 - DNA, 73 bp, Artificial Sequence
  Description of Artificial Sequence Synthetic oligonucleotide
ctgaatgcatgggtgaaagttgtcagagacaccgcagtgctggatgtgggggatgcctacttctcagtgagag

SEQ ID NO: 86 - DNA, 70 bp, Artificial Sequence
  Description of Artificial Sequence Synthetic oligonucleotide
atgcctacttctcagtgagagctaagacttatctggtcaaactctggtaccagttgagggctgacactcg

SEQ ID NO: 87 - DNA, 79 bp, Artificial Sequence
  Description of Artificial Sequence Synthetic oligonucleotide
cagttgagggctgacactcgtctttacaacactgtggccacccttaggaccaaggctcttctggacactggagcagatg

SEQ ID NO: 88 - DNA, 76 bp, Artificial Sequence
  Description of Artificial Sequence Synthetic oligonucleotide
cttctggacactggagcagatgacactgtgagggctaagaccctgctgtggaagggagagggagcagttaggactg

SEQ ID NO: 89 - DNA, 66 bp, Artificial Sequence
  Description of Artificial Sequence Synthetic oligonucleotide
aagggagagggagcagttaggactgatgcttacatctaccagtatatggatgaccttagactcgag

SEQ ID NO: 90 - DNA, 34 bp, Artificial Sequence
  Description of Artificial Sequence Synthetic oligonucleotide
catgaactggctctttcaagtaggaattccactg

SEQ ID NO: 91 - DNA, 35 bp, Artificial Sequence
  Description of Artificial Sequence Synthetic oligonucleotide
gcagtggaattcctacttgaaagagccagttcatg

SEQ ID NO: 92 - DNA, 34 bp, Artificial Sequence
  Description of Artificial Sequence Synthetic oligonucleotide
ctatatgctcgagtctaaggtcatccatatactg

SEQ ID NO: 93 - DNA, 73 bp, Artificial Sequence
  Description of Artificial Sequence Synthetic oligonucleotide
attcgtgctggatgtgggagatgcctacttctcagtgagagctgacacctacctgaatgcctgggtgaaggtg

SEQ ID NO: 94 - DNA, 72 bp, Artificial Sequence
  Description of Artificial Sequence Synthetic oligonucleotide
acctgaatgcctgggtgaaggtggtcagagccaagacctacctggtgaagctgtggtaccagctgaggacag

SEQ ID NO: 95 - DNA, 66 bp, Artificial Sequence
  Description of Artificial Sequence Synthetic oligonucleotide
agctgtggtaccagctgaggacagatgcctccctggtgaagcatcacatgtatgtgagagacacag

SEQ ID NO: 96 - DNA, 58 bp, Artificial Sequence
  Description of Artificial Sequence Synthetic oligonucleotide
agcatcacatgtatgtgagagacacagcctacatctaccagtacatggatgacctgag

SEQ ID NO: 97 - DNA, 31 bp, Artificial Sequence
  Description of Artificial Sequence Synthetic oligonucleotide
gagaatgaattcgtgctggatgtgggagatg

SEQ ID NO: 98 - DNA, 37 bp, Artificial Sequence
  Description of Artificial Sequence Synthetic oligonucleotide
ctatatctcgagtctcaggtcatccatgtactggtag

SEQ ID NO: 99 - DNA, 73 bp, Artificial Sequence
  Description of Artificial Sequence Synthetic oligonucleotide
attcctgcttgacacaggagctgatgacacagtgaggacagatgccagcctgtataacacagtggccaccctg

SEQ ID NO: 100 - DNA, 70 bp, Artificial Sequence
  Description of Artificial Sequence Synthetic oligonucleotide
agcctgtataacacagtggccaccctgagagctgacacctacctgaaggagcctgtgcatggagtgagag

SEQ ID NO: 101 - DNA, 71 bp, Artificial Sequence
  Description of Artificial Sequence Synthetic oligonucleotide
agcctgtgcatggagtgagagctaagaccctcctgtggaagggagagggagcagtgagaaccaaggcagtg

SEQ ID NO: 102 - DNA, 55 bp, Artificial Sequence
  Description of Artificial Sequence Synthetic oligonucleotide
agcagtgagaaccaaggcagtgctggctgaggccatgtcccaggtgagactcgag

SEQ ID NO: 103 - DNA, 31 bp, Artificial Sequence
  Description of Artificial Sequence Synthetic oligonucleotide
gagaatgaattcctgcttgacacaggagctg

SEQ ID NO: 104 - DNA, 28 bp, Artificial Sequence
  Description of Artificial Sequence Synthetic oligonucleotide
ctagatctcgagtctcacctgggacatg

SEQ ID NO: 105 - DNA, 66 bp, Artificial Sequence
  Description of Artificial Sequence Synthetic primer
aattcccctaggaccctgaatgcctgggtgagagctaagaccagacctaacaataacacaaggaag

SEQ ID NO: 106 - DNA, 74 bp, Artificial Sequence
  Description of Artificial Sequence Synthetic primer
accagacctaacaataacacaaggaagtccatcagagacacagccttccctgtgagaccacaggtgcctctgag

SEQ ID NO: 107 - DNA, 67 bp, Artificial Sequence
  Description of Artificial Sequence Synthetic primer
agaccacaggtgcctctgaggagaaccaaggcccaccctgtgcatgctggccctattgccagagctg

SEQ ID NO: 108 - DNA, 68 bp, Artificial Sequence
  Description of Artificial Sequence Synthetic primer
atgctggccctattgccagagctgatacagcacccactaaggccaaaaggagagtggtcaggctcgag

SEQ ID NO: 109 - DNA, 35 bp, Artificial Sequence
  Description of Artificial Sequence Synthetic primer
gtataagaattctcccctaggaccctgaatgcctg

SEQ ID NO: 110 - DNA, 32 bp, Artificial Sequence
  Description of Artificial Sequence Synthetic primer
ctatatgctcgagcctgaccactctccttttg

SEQ ID NO: 111 - DNA, 69 bp, Artificial Sequence
  Description of Artificial Sequence Synthetic primer
aattccaagcctgtggtctccacacagctgcttctcagggccaagaccttccctgtgagaccccaagtg

SEQ ID NO: 112 - DNA, 74 bp, Artificial Sequence
  Description of Artificial Sequence Synthetic primer
accttccctgtgagaccccaagtgccactgagaagggctgatacacagcccaggagtgacacccatgtgttcag

SEQ ID NO: 113 - DNA, 70 bp, Artificial Sequence
  Description of Artificial Sequence Synthetic primer
aggagtgacacccatgtgttcagaaccaaggccattcctaggagaattaggcagggcctgagagatacag

SEQ ID NO: 114 - DNA, 63 bp, Artificial Sequence
  Description of Artificial Sequence Synthetic primer
attaggcagggcctgagagatacagctacacctcaggacctgaacaccatgctgagactcgag

SEQ ID NO: 115 - DNA, 33 bp, Artificial Sequence
  Description of Artificial Sequence Synthetic primer
gtcctggaattcaagcctgtggtctccacacag

SEQ ID NO: 116 - DNA, 31 bp, Artificial Sequence
  Description of Artificial Sequence Synthetic primer
ctatatgctcgagtctcagcatggtgttcag

SEQ ID NO: 117 - DNA, 18 bp, Artificial Sequence
  Description of Artificial Sequence Synthetic oligonucleotide
atgcagtggaactccaca

SEQ ID NO: 118 - DNA, 18 bp, Artificial Sequence
  Description of Artificial Sequence Synthetic oligonucleotide
ggggctggagcaggagct

SEQ ID NO: 119 - DNA, 24 bp, Artificial Sequence
  Description of Artificial Sequence Synthetic oligonucleotide
gattacaaggacgacgacgacaag

SEQ ID NO: 120 - DNA, 30 bp, Artificial Sequence
  Description of Artificial Sequence Synthetic oligonucleotide
gaattcctgcaggctagcagatctctcgag

SEQ ID NO: 121 - DNA, 66 bp, Artificial Sequence
  Description of Artificial Sequence Synthetic oligonucleotide
gactacaaagaccacgacggtgattataaagatcacgacattgattacaaggacgacgacgacaag

SEQ ID NO: 122 - DNA, 5693 bp, Artificial Sequence
  Description of Artificial Sequence Synthetic polynucleotide
aacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaattcagctagcgatatccccgggctcgagcatataggaccagggagagcatttgtggtacctctagaagaagcttgggatccgtcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaata

SEQ ID NO: 123 - DNA, 5672 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcctgcaggctagcagatctctcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 124 - DNA, 5714 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcctgcaggctagcagatctctcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 125 - DNA, 399 bp, Artificial Sequence
  Description of Artificial Sequence Synthetic polynucleotide
atgcagtggaactccacacccggggctggagcaggagctgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatg

SEQ ID NO: 126 - DNA, 447 bp, Artificial Sequence
  Description of Artificial Sequence Synthetic polynucleotide
caggccatgcagtggaactccacacccggggctggagcaggagctgactacaaagaccacgacggtgattataaagatcacgacattgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatg

SEQ ID NO: 127 - DNA, 5969 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 128 - DNA, 6011 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 129 - DNA, 5846 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcgtgctggatgtgggagatgcctacttctcagtgagagctgacacctacctgaatgcctgggtgaaggtggtcagagccaagacctacctggtgaagctgtggtaccagctgaggacagatgcctccctggtgaagcatcacatgtatgtgagagacacagcctacatctaccagtacatggatgacctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 130 - DNA, 5843 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcctgcttgacacaggagctgatgacacagtgaggacagatgccagcctgtataacacagtggccaccctgagagctgacacctacctgaaggagcctgtgcatggagtgagagctaagaccctcctgtggaagggagagggagcagtgagaaccaaggcagtgctggctgaggccatgtcccaggtgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 131 - DNA, 5888 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcgtgctggatgtgggagatgcctacttctcagtgagagctgacacctacctgaatgcctgggtgaaggtggtcagagccaagacctacctggtgaagctgtggtaccagctgaggacagatgcctccctggtgaagcatcacatgtatgtgagagacacagcctacatctaccagtacatggatgacctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 132 - DNA, 5885 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcctgcttgacacaggagctgatgacacagtgaggacagatgccagcctgtataacacagtggccaccctgagagctgacacctacctgaaggagcctgtgcatggagtgagagctaagaccctcctgtggaagggagagggagcagtgagaaccaaggcagtgctggctgaggccatgtcccaggtgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 133 - DNA, 5849 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattctcccctaggaccctgaatgcctgggtgagagctaagaccagacctaacaataacacaaggaagtccatcagagacacagccttccctgtgagaccacaggtgcctctgaggagaaccaaggcccaccctgtgcatgctggccctattgccagagctgatacagcacccactaaggccaaaaggagagtggtcaggctcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 134 - DNA, 5846 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcaagcctgtggtctccacacagctgcttctcagggccaagaccttccctgtgagaccccaagtgccactgagaagggctgatacacagcccaggagtgacacccatgtgttcagaaccaaggccattcctaggagaattaggcagggcctgagagatacagctacacctcaggacctgaacaccatgctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 135 - DNA, 5891 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattctcccctaggaccctgaatgcctgggtgagagctaagaccagacctaacaataacacaaggaagtccatcagagacacagccttccctgtgagaccacaggtgcctctgaggagaaccaaggcccaccctgtgcatgctggccctattgccagagctgatacagcacccactaaggccaaaaggagagtggtcaggctcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 136 - DNA, 5810 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcaagcctgtggtctccacacagctgcttctcagggccaagaccttccctgtgagaccccaagtgccactgagaagggctgatacacagcccaggagtgacacccatgtgttcagaaccaaggccattcctaggagaattaggcagggcctgagagatacagctacacctcaggacctgaacaccatgctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcgga